(19) United States
(12) Patent Application Publication
(10) Pub. No.: US 2025/0321237 A1
(43) Pub. Date: Oct. 16, 2025

(54) ASSAY FOR RAPID PROTEIN MULTIMER DETECTION, CHARACTERIZATION AND QUANTIFICATION

(71) Applicant: Western Michigan University Homer Stryker M.D. School of Medicine, Kalamazoo, MI (US)

(72) Inventors: Michael Gutknecht, Kalamazoo, MI (US); Thomas L. Rothstein, Kalamazoo, MI (US)

(73) Assignee: Western Michigan University Homer Stryker M.D. School of Medicine, Kalamazoo, MI (US)

(21) Appl. No.: 18/862,082

(22) PCT Filed: May 1, 2023
(86) PCT No.: PCT/US2023/020576

§ 371 Date: Oct. 31, 2024

Related U.S. Application Data
(60) Provisional application No. 63/337,128, filed on May 1, 2022.

(51) Int. Cl. G01N 33/68; G01N 33/542; G01N 33/543; G01N 33/545

(52) U.S. Cl. CPC G01N 33/6896; G01N 33/542; G01N 33/54386; G01N 33/545; G01N 2800/28

(57) ABSTRACT

A number of protein aggregation diseases are associated with accumulation of misfolded proteins, which are known as protein aggregates, including, but not limited to, neurodegenerative and non-degenerative diseases and disorders. The present disclosure provides an assay, compositions and kits for the qualitative and quantitative assessment of aggregated proteins in solution using a microparticle immunocapture assay that combines the advantages inherent to a specific first and second capture moiety that binds specifically to an aggregated protein which can reveal at the same time the amount and the size of aggregates measured in a sample, fluid, tissue, cavity, or pharmacological product.

### CROSS REFERENCE TO RELATED APPLICATIONS

This PCT application claims priority to U.S. Provisional Application No. 63/337,128, filed May 1, 2022. The entire contents of this application is incorporated herein by reference.

### TECHNICAL FIELD

Without limitation, some embodiments comprise methods, kits, compositions and systems for qualitative and quantitative analysis of pathological multimeric protein aggregates associated with disease.

### BACKGROUND

A number of neurodegenerative and other diseases are associated with accumulation of damaged, misfolded proteins that form pathological insoluble deposits, including Alzheimer's disease (AD) which is associated with the accumulation of Amyloid beta (AB) peptide, and/or Tau protein; Parkinson's disease (PD) and Lewy Body Dementia (associated with α-synuclein); Huntington's disease (HD) (associated with Huntingtin with tandem glutamine repeat expansion); amyotropic lateral sclerosis (ALS) (associated with Superoxide dismutase 1 and/or TDP-43 and/or FUS and/or other disordered proteins); Multiple tauopathies including fronto-temporal dementia (associated with Tau protein); Spongiform encephalopathies (associated with prion proteins); Familial amyloid polyneuropathy (associated with transthyretin variants); and chronic traumatic encephalopathy, and other illnesses including primary systemic amyloidosis (associated with immunoglobulin light chain); secondary systemic amyloidosis (associated with serum amyloid A); senile systemic amyloidosis (associated with transthyretin); hemodialysis-related amyloidosis (associated with β2-microglobulin); lysozyme systemic amyloidosis (associated with lysozyme); type II diabetes (associated with islet amyloid polypeptide or amylin); hereditary renal amyloidosis (associated with fibrinogen); cataract disease (associated with crystallin); retinitis pigmentosa (associated with rhodopsin), sickle cell anemia and thalassemia (associated with variant hemoglobin) and other related diseases for which protein oligomers, multimers, fibrils, or aggregates are pathogenically and/or pathologically involved (Sacchettini and Kelly, 2002).

Protein aggregation is the formation of multimer assemblies from disordered mutant or damaged protein monomers. In such situations, established control mechanisms fail to sufficiently induce proper protein refolding or to adequately remove unrecoverable proteins for degradation via proteosome and autophagy mechanisms (Mogk et al., 2018; Tanaka et al., 2014). The molecules and pathways that maintain protein homeostasis, or proteostasis, have been intensely investigated given that dysregulated aggregate accumulation is deleterious to cellular viability. In addition to direct toxicity, protein aggregates are thought to be harmful through loss-of-function related to deficient physiology of proteins now aggregated and nonfunctional, and/or to exhaustion of remediating mechanisms.

The connection between aggregate formation and disease is widespread, encompassing neuro-pathologies such as Alzheimer's disease (amyloid, tau), Parkinson's disease (alpha-synuclein), Huntington's disease (huntingtin), prion propagated disease (PrP), amyotrophic lateral sclerosis (TDP-43, SOD1, FUS, and more), as well as disease in other tissues, such as the heart (cardiac amyloidosis) and pancreas (type II diabetes, islet cell IAPP), as outlined in 0002. This is most strikingly illustrated in brain sections from Alzheimer's disease patients, where immunohistochemical detection and visualization of beta-amyloid and tau protein deposits scattered in large abundance are routinely observed and correlate with disease severity (Aguzzi et al., 2010; Haass et al., 2007).

Although the means by which protein aggregates damage cells remain uncertain, the need for robust and accurate quantitative methods to distinguish between protein monomers and aggregated multimers, and to evaluate the amount and size of protein aggregates, is clear yet has not been fully addressed (Cox et al., 2020). This applies to clinical diagnostics, where plasma neuro-protein aggregate detection is rapidly becoming more accepted as an indicator of neurodegenerative disease, as well as to basic research, where the proteostatic activity of chemical compounds and biological molecules is determined (Lindquist et al., 2011; Palmqvist et al., 2020; Shahnawaz et al., 2017; Tokuda et al., 2010). It also applies to pharmacologic preparations of therapeutic proteins where it is necessary to determine whether proteins in such products aggregate and to determine the time period during which this occurs and exceeds an acceptable level (shelf-life and expiration-date). Several methods and tools have been adapted to evaluate protein aggregation, including electron microscopy, light scattering, electrophoretic migration, and enzyme-linked immunosorbent assay (ELISA), among others, with each having its own set of advantages and limitations (Bagriantsev et al., 2006; Chaudhuri et al., 2014; den Engelsman et al., 2011; El-Agnaf et al., 2006; Mahler et al., 2009). However, none combine quantification of degree of aggregation (e.g., ELISA) and size of aggregates (e.g., electrophoresis). In addition, none combine these characteristics with target specificity, ease of use, speed of results, and availability of needed instrumentation.

### SUMMARY

The present disclosure provides compositions, kits and methods for the quantitative and qualitative measurement, assessment and analysis of pathological multimeric protein aggregates involved in several diseases such as proteinopathies; and, compositions, kits and methods for the quantitative and qualitative measurement, assessment and analysis of multimeric protein aggregates that form in pharmacologic preparations that contain protein.

In a first aspect, a method and assay for the quantitative and qualitative measurement, assessment and analysis of multimeric protein aggregates includes:
a. providing a capture substrate comprising a microparticle conjugated to a first capture moiety;
b. blocking the capture substrate non-specific binding sites;
c. incubating the capture substrate with a test sample suspected of having the aggregated protein for a period of time sufficient for the aggregated protein to specifically bind to the first capture moiety; wherein the first capture moiety binds specifically to the aggregate protein if present, thereby forming a capture complex on the capture substrate,
d. incubating the capture substrate with a second capture moiety that specifically binds to the aggregated protein, wherein the second capture moiety is coupled to a signalling moiety, and wherein the signaling moiety comprises a detectable label; and
e. determining the amount of detectable label present on the surface of the capture substrate. In another aspect, compositions and kits are provided for the performance of the above described methods.

In another aspect, compositions and kits are provided for the performance of the above described methods. In some embodiments, a kit may comprise: (a) a plurality of microparticles, the microparticles comprising a first capture moiety that specifically binds an aggregate protein of interest; (b) a composition comprising a second capture moiety that specifically binds to the aggregate protein, wherein the second capture moiety is conjugated to a signalling moiety comprising a detectable label or a first binding partner, and (c) optionally a detectable label that is coupled to a second binding partner that specifically binds to the first binding partner, wherein the first and second capture moieties bind to the same epitope or antigen, or amino acid sequence present on the aggregated protein or other molecule of interest. In various embodiments, the first binding partner and the second binding partner comprises biotin, avidin or streptavidin. The second capture moiety may be conjugated directly to a signal molecule rather than to a signalling moiety that is then bound by a signal molecule.

### BRIEF DESCRIPTION OF THE DRAWINGS

Some embodiments will now be described, by way of example only and without waiver or disclaimer of other embodiments, with reference to the accompanying drawings, in which:

FIG. 1 depicts an assay schematic. FIG. 1A. Monoclonal antibodies (mAb) specific for an aggregation prone protein are adsorbed onto super active aldehyde sulfate microbeads. The remaining active sites on beads are blocked with irrelevant protein such as bovine serum albumin (BSA). FIG. 1B. The bead-mAb combination is incubated with the target protein. Illustrated are examples for solutions containing non-aggregated protein monomer only (top row) or monomers and multimers (bottom row). mAb-target protein binding occurs at a specific site (green box). All antibody binding sites on bound monomers are saturated, whereas unoccupied binding sites exist on bound multimers (green asterisks). FIG. 1C. The bead-mAb-target protein complex is incubated sequentially with the identical monoclonal antibody (biotinylated) and streptavidin fluorophore, followed by fluorescence detection and quantitation.

FIG. 2. Characterization and antibody binding properties of aldehyde sulfate beads. FIG. 2A. Microscopic brightfield image of unlabeled beads. Yellow arrowheads denote the most prevalent, comparatively smaller diameter beads. Turquoise arrowheads denote the less numerous larger diameter beads. 20× magnification. FIG. 2B. Flow cytometry dot plot of buffer only (left) or buffer+ beads (right). Three gated populations distinguished by the physical parameters of size (FSC-A) and complexity/granularity (SSC-A) are shown. The majority of beads (77.1%) fall within the low FSC-A gate. FIG. 2C. Beads were first incubated with the indicated PBS-based blocking buffer preparation for 60 min, then with IgG phycoerythrin (PE) (0.0025 μg/μl) for 30 min, and subsequently analyzed by flow cytometry. Dot plots display the percentage of PE+ beads and geometric mean intensity (GMI) PE of the low FSC-A bead population. FIG. 2D. Beads labeled with IgG AF594 were blocked with BSA, mixed at equal number with sorted murine B cells, and imaged. 20× magnification. FIG. 2E. Beads (1.0 μl bead stock/100 μl PBS) were incubated with titrated biotinylated anti-Aβ42 monoclonal antibody mAb (clone 12F4), blocked, incubated with an equivalent amount of streptavidin (SA)-phycoerythrin (PE), washed, and analyzed by flow cytometry. Dot plots display the gating scheme utilized to identify the low FSC-A singlet AS bead population for analysis. From left to right, the bead population was identified from all acquired events (1.; 93%) using FSC-A and SSC-A. The low FSC-A population (2.; black arrowhead; 71%) was subjected to FSC (3.; 100%) and SSC (4.; 100%) pulse width analysis to discriminate single beads from bead aggregates. The GMI PE of the resultant bead population (5.) was determined for all samples (example shown for the SA PE only sample; gate indicates the PE+ bead population). FIG. 2F. Overlay of representative dot plots showing the PE intensity and gated PE+ beads for samples incubated with the indicated amount of 12F4 mAb. The color table to the right shows the GMI PE value for each of the 12F4 mAb titrations shown in the dot plot. FIG. 2G. Two graphs represent the bead GMI PE for all titrated 12F4 samples (left) and the most dilute 12F4 mAb samples only (right). FIG. 2H. Flow cytometry dot plots (bottom row) display the bead PE intensity for beads incubated with 0.5 μg-0.031 μg 12F4 mAb, with beads that exhibited sub-maximal signal intensity highlighted by black arrowheads. I. Beads prepared at 0.1 μl bead stock/100 μl PBS were incubated with titrated biotinylated-12F4 mAb and analyzed by flow cytometry. The graph represents the bead GMI PE for all titrations. Note: Due to the limitations of using a value of 0 on a log scale, the data points for the lowest amount of amyloid on all graphs represent 0 μg amyloid.

FIG. 3. Determination of 12F4 mAb specificity and effective assay working conditions. FIG. 3A. Lyophilized monomer Aβ42 was resuspended at 0.25 μg Aβ42/1.0 μl assay buffer A and titrated as indicated. After incubation with 1.0 μl aldehyde sulfate beads (100 μl total volume buffer) for 60 min, the preparations were blocked, washed, incubated with biotinylated 12F4 mAb, washed, incubated with SA PE, washed, and analyzed by flow cytometry. Representative dot plots display both the PE+ bead gate and GMI PE for all beads. The graph displays the bead GMI for all Aβ42 titrations. FIG. 3B. Fluorescently labeled monomeric Aβ42 (Aβ42 HL488) titrated at the indicated amount was incubated with beads for 45 min to allow adsorption (0.1 μl bead stock/100 μl total volume), and the beads were subsequently analyzed by flow cytometry. The bead GMI HL488 is presented for all titrations. FIG. 3C. Aβ42 monomer solution was kept on ice (0 min), or incubated at 37° C./1000 rpm for the time indicated (15 min, 60 min, 480 min) to induce multimerization. The resultant Aβ42 samples were prepared at the indicated amounts and resolved by native PAGE, stained with Coomassie R250, and imaged. Noted are the migrating distances of the stock monomer (black arrowhead), small multimers (black line), large multimers (red line), and large aggregates that failed to migrate (red arrowhead). FIG. 3D. Beads activated with either 12F4 mAb or buffer only were blocked, incubated with the indicated amount of Aβ42 60 min multimers (100 μl total volume), washed, incubated with biotinylated 12F4 mAb (detection), washed, incubated with SA PE, washed, and analyzed by flow cytometry. Representative dot plots with the gated PE+ population are shown. The graph displays the bead GMI PE for 12F4 mAb-activated beads (black) and buffer only beads (grey). FIG. 3E. 12F4 mAb-loaded beads were incubated with titrated Aβ42 60 min multimers in the indicated reaction volumes (solid black circle, 40 μl; open black circle, 200 μl; open black square, 1000 μl), followed by the standard wash, detection, and flow cytometry analysis steps as above. The graph displays the bead GMI PE of all beads. FIG. 3F. 12F4 mAb-activated beads were incubated with or without 0.1 μg Aβ42 60 min aggregates in the presence of the indicated amount of Aβ42 monomer, followed by wash, detection, and flow cytometry analysis. The bead PE GMI for beads incubated with (black) or without (grey) aggregate are presented. FIG. 3G. 12F4 mAb-activated beads were incubated with titrated Aβ42 60 min aggregate, followed by detection and flow cytometry analysis. The dot plots display representative examples of bead GMI PE and percentage of PE+ beads (indicated by gate). FIG. 3H. The GMI PE for all titrations (left) and the most dilute samples only (right) are presented. The lowest Aβ42 amount that displayed significance compared to no amyloid control is indicated. FIG. 3I. The percentage PE+ beads for all titrations (left) and the most dilute samples only (right) are presented. Indicated is the lowest Aβ42 amount that displayed significance compared to no amyloid control. N=3 for FIG. 3G, FIG. 3H, and FIG. 3I. Errors bars indicate standard deviation of the mean. *P<0.05. Note: Due to the limitations of using a value of 0 on a log scale, the data points for the lowest amount of amyloid on all graphs represent 0 μg amyloid.

FIG. 4. Assay detection of Aβ42 oligomers and protofibrils. FIG. 4A. To isolate Aβ42 oligomers and protofibrils, Aβ42 monomers were incubated at 4° C. for 120 hours to induce multimerization, and the resultant samples were subjected to size exclusion chromatography using a SEC 650 column. Equivalent volumes of the indicated fractions were resolved by PAGE, and the gel was then stained by Coomassie R250 and imaged. Indicated are the fractions enriched for protofibrils and oligomers. FIG. 4B. Pooled oligomer fractions were titrated at the indicated amounts and then analyzed by the bead assay. The PE+ population and GMI PE of all beads for representative samples are indicated in the dot plots. The graphs display the GMI PE (left) and percentage of PE+ beads (right) for all analyzed samples. FIG. 4C. Pooled protofibril fractions were titrated at the indicated amounts and then analyzed by the bead assay. The PE+ population and GMI PE of all beads for representative samples are indicated in the dot plots. The graphs display the GMI PE (left) and percentage of PE+ beads (right) for all analyzed samples. Note: Due to the limitations of using a value of 0 on a log scale, the data points for the lowest amount of amyloid on all graphs represent 0 μg amyloid.

FIG. 5. Quantitation of Aβ42 multimer size. FIG. 5A. Aβ42 monomer solution was kept on ice (0 min), or incubated at 37° C./1000 rpm for the indicated amount of time to induce aggregation. An equivalent amount of protein for each sample was resolved by native PAGE, stained with Coomassie R250, and imaged. Noted are the migrating distances of the stock monomer (black arrowhead), small multimers (black line), large multimers (red line), and large aggregates that failed to migrate (red arrowhead). FIG. 5B. Aldehyde sulfate beads were activated with 12F4 mAb, blocked, incubated with 0.1 μg of the Aβ42 preparations described above, washed, detected, washed, and then analyzed by flow cytometry. Buffer only condition was used first to define PE+ beads. The 60 min aggregate sample (right-most dot plot) was used second to define four bead quadrants (equivalent proportions) based on relative PE fluorescence intensity. The gates were then applied to all samples and the percentage of beads in each of the four populations was determined. Dot plots for the indicated sample preparations are presented. FIG. 5C. The percentage of beads in each population, designated in silver (PE^{dim}), yellow (PE^{low}), orange (PE^{med}), and red (PE^{high}), are presented. Data for the shortest aggregation time only (0-15 min) are shown in the inset graph. FIG. 5D. Aβ42 monomers were incubated for 30 or 60 min at 37° C./1000 rpm and then subjected to ultracentrifugation. Equivalent Aβ42 amount from the 30 min supernatant (30 min SUP), the 60 min pellet (60 min PEL), and Aβ42 monomer were resolved by native PAGE, stained by Coomassie R250, and imaged. FIG. 5E. Beads were activated with 12F4 mAb, blocked, incubated with 0.1 μg of the Aβ42 preparations described above, washed, detected, washed, and then analyzed by flow cytometry. A gating scheme similar to that described above was applied. Here, the 60 min PEL PE+ bead population was utilized to derive four quadrants, and the gates were then applied to all samples. Displayed are the dot plots with applied gates. FIG. 5F. The graph displays the percentage of beads in each population, designated by color as above. Note: Data presented for the aggregation time course (FIG. 5A-5C.) and the isolation of Aβ42 species by differential ultracentrifugation (FIG. 5D-5F.) are representative of at least two independent experiments.

FIG. 6. Detection and measurement of aggregated alpha-synuclein (αS) in human cerebrospinal fluid (CSF). FIG. 6A. Monomeric αS was aggregated according to the manufacturer's instructions (37° C./1000 rpm) and analyzed by native PAGE to confirm aggregation state. Displayed on the coomassie-stained gel are titrated (starting 1.25 μg αS, with 1:2 dilutions) aS monomer (left lanes) and S aggregate (right lanes). Use of protein molecular weight standards (indicated M., lanes 1 and 6) is for the purposes of sample lane separation and gel orientation only and not to determine sample molecular weight. The red arrowhead indicates the accumulation of aggregated protein in the &S aggregate sample, which is not visible in the αS monomer sample. FIG. 6B. Aldehyde sulfate beads were incubated in either PBS alone or PBS+anti-αS mAb MJFR1, and subsequently blocked, washed, and incubated with titrated αS monomer or αS aggregate. Following detection with MJFR1-PE mAb, the beads were analyzed for PE intensity by flow cytometry. The graph displays the GMI PE of beads without mAb activation (left) or with MJFR1 mAb activation (right) for all titrated samples of αS monomer (blue circles) and αS aggregate (red circles), or no αS (buffer, black circles). FIG. 6C. Human CSF was prepared at the indicated concentration in PBS (25.0%-0.4% CSF) and spiked with an equivalent amount (0.5 μg) of either αS monomer or αS aggregate. The samples were then incubated with MJFR1-activated beads, and following block, washes, and detection, the beads were analyzed for PE intensity by flow cytometry. The graph displays the GMI PE for all beads analyzed for each condition. Open circles indicate samples prepared without αS in either buffer alone (black) or 25% CSF (orange; overlaps with open black circle). Indicated by solid circles are buffer alone+αS (black), or titrated CSF+αS (orange). FIG. 6D. Aggregated αS was titrated at the indicated amount and combined with 1% human CSF. Following incubation with MJFR1 mAb-activated beads and subsequent detection, the bead fluorescence was determined by flow cytometry. Dot plots for buffer (top row) or buffer/CSF (bottom row) conditions for each sample preparation are presented, with the GMI PE presented in the lower left. FIG. 6E. The graph displays the bead GMI PE for all titrated samples. Open circles represent samples prepared without αS in either buffer alone (black) or 1% CSF (orange; overlaps with open black circle). Depicted by solid circles are buffer alone+αS (black), or 1% CSF+αS (orange). The inset graph displays the most dilute αS preparations only. Indicated is the as amount at which a statistically significant difference in PE intensity above buffer alone conditions was observed. FIG. 6F. Aggregated αS was prepared at 0.1 μg in either buffer or 50% CSF/buffer. The samples were then titrated 1:4 in buffer and subsequently analyzed by the bead assay. Indicated by dot plot are the percentage of PE+ beads and GMI PE for buffer/no αS (column one), buffer/0.1 μg αS (column two, row one), and 50% CSF/0.1 μg αS (column two, row two) conditions. The graphs display the GMI PE intensity (left) and the percentage of PE+ beads (right) at the indicated αS amount for buffer (black) and CSF (orange) conditions. The open black circles represent buffer only conditions with no as added. FIG. 6G. Parkinson's disease patient CSF (PD CSF) obtained from two commercial sources and normal CSF were titrated in buffer at the indicated amount and analyzed by the bead assay. The gated populations indicate the percentage of PE+ beads at each titration as compared to buffer only conditions (far left dot plot). Note: Data presented in FIGS. 6B and 6C are representative of at least two independent experiments. For FIGS. 6D and 6E, N=4 independent experiments from four separate human CSF donors. Errors bars indicate standard error of the mean. *P<0.05.

### DETAILED DESCRIPTION

### Definitions

The following set of definitions is merely exemplary in nature and is not intended to limit the present disclosure, application, or uses.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Thus, although the example above involved monoclonal antibodies (mAb) specific for an aggregation prone protein, aggregation of a particular ligand could be evaluated by coupling its cognate receptor to super active aldehyde sulfate microbeads (or other beads suitable for coupling) combined with detection by the same receptor coupled to an indicator agent such as a fluorophore, or, vice versa, aggregation of a particular receptor could be evaluated by coupling its cognate ligand to super active aldehyde sulfate microbeads (or other beads suitable for coupling) combined with detection by the same ligand coupled to an indicator agent such as a fluorophore. Similarly, aggregation of proteins that bind nucleic acid DNA or RNA sequences could be detected by coupling the specific nucleic acid DNA or RNA sequence to microbeads and evaluating with a fluorophore or other indicator-coupled nucleic acid DNA or RNA sequence. Specific examples are provided for illustrative purposes of how to make and use the compositions and methods of this technology and, unless explicitly stated otherwise, are not intended to be a representation that given embodiments of this technology have, or have not, been made or tested. The following definitions and non-limiting guidelines must be considered in reviewing the description of the technology set forth herein.

The headings (such as “Introduction” and “Summary”) and sub-headings used herein are intended only for general organization of topics within the present technology and are not intended to limit the disclosure of the present technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the Introduction is intended merely to provide a general summary of assertions made by the authors of the references and does not constitute an admission as to the accuracy of the content of such references. All references cited in the “Description” section of this specification are hereby incorporated by reference in their entirety.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

Although the open-ended term “comprising,” as a synonym of terms such as including, containing, or having, is used herein to describe and claim the present disclosure, the disclosure, or embodiments thereof, may alternatively be described using more limiting terms such as “consisting of” or “consisting essentially of” the recited ingredients.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, an exemplary neurodegenerative proteinopathy is a neurodegenerative disease or condition in which at least one physiological event that contributes or is associated with the neurodegenerative proteinopathy is the presence of misfolded proteins in the brain, neurons (e.g., neurons of the central or peripheral nervous system), and/or spinal column, of the subject with the neurodegenerative disease or condition. Examples of neurodegenerative proteinopathies that can be evaluated, leading to treatment or prevention, with the compositions of the present disclosure include, but are not limited to, Alzheimer's disease (AD) (associated with Amyloid beta (Aβ) peptide; Tau), Parkinson's disease (PD) (associated with α-synuclein), Huntington's disease (HD) (associated with Huntingtin with tandem glutamine repeat expansion), amyotropic lateral sclerosis (ALS) (associated with Superoxide dismutase 1, and/or TDP-43, and/or FUS and/or other proteins), Multiple tauopathies (associated with Tau protein), Spongiform encephalopathies (associated with prion proteins), Familial amyloidotic polyneuropathy (associated with transthyretin) and, chronic traumatic encephalopathy, as outlined in 0002.

By isolated and “substantially pure” is meant a protein or polypeptide that has been separated and purified to at least some degree from the components that naturally accompany it. Typically, a polypeptide is substantially pure when it is at least about 60%, or at least about 70%, at least about 80%, at least about 90%, at least about 95%, or even at least about 99%, by weight, free from the proteins and naturally occurring organic molecules with which it is naturally associated. For example, a substantially pure protein or polypeptide may be obtained by extraction from a natural source, by expression of a recombinant nucleic acid in a cell that does not normally express that protein, or by chemical synthesis.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “recombinant” as used herein to describe a nucleic acid molecule, means a polynucleotide of genomic, mRNA, cDNA, viral, semisynthetic, and/or synthetic origin, which, by virtue of its origin or manipulation, is not associated with all or a portion of the polynucleotide with which it is associated in nature, thus it is non-natural. The term recombinant as used with respect to a protein or polypeptide, means a polypeptide produced by expression of a recombinant polynucleotide. The term recombinant as used with respect to a host cell means a host cell into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

By “wild type” or “WT” or “native” herein is meant an amino acid sequence or a nucleotide sequence that is found in nature, including allelic variations.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

The term “conjugated” generally means to be joined together, to be coupled, or to act or operate as if joined. Usually, conjugation occurs by covalent linkage or ionic interaction

As used herein, “subject” refers to an animal, including, but not limited to, a primate (e.g., human). The terms “subject” and “patient” are used interchangeably herein

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “detection” includes any means of detecting, including direct and indirect detection.

The term “specific binding,” “specifically binds,” and the like, refer to the preferential binding to a molecule relative to other molecules or moieties in a solution or reaction mixture. In some embodiments, the affinity between moiety, such as an antibody, or an antigen binding portion thereof, and the target analyte to which it specifically binds when they are specifically bound to each other in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less.

“Affinity” refers to the strength of binding, increased binding affinity being correlated with a lower K_{d}. As such, “binds specifically” or “specifically binds” is not meant to preclude a given binding member from binding to more than one analyte of interest. For example, antibodies that bind specifically to an aggregate protein of interest may be capable of binding other polypeptides at a weak, yet detectable, level (e.g., 10% or less of the binding shown to the polypeptide of interest). Such weak binding, or background binding, is readily discernible from the specific antibody binding to the aggregated protein of interest, e.g., by use of appropriate controls.

As used herein, the term “antibody” or “antibodies” includes antibodies or immunoglobulins of any isotype, fragments of antibodies which retain specific binding to antigen, including, but not limited to, Fab, F(ab′)₂, Fv, scFv, bi-specific-scFv, diabody, Fd, and Fc fragments, chimeric antibodies, humanized antibodies, fully human antibodies, single-chain antibodies, and fusion proteins including an antigen-binding portion of an antibody and a non-antibody protein. Monospecific antibodies and their antigen binding fragments thereof are antibodies that bind to only a single antigen or epitope or protein sequence on the target protein. For example, polyclonal antibodies are not monospecific antibodies of the present disclosure because by their very nature, they bind to multiple epitopes on a protein. Monoclonal antibodies are one example of a monospecific antibody. Other antibody types may also be monospecific.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions may be joined by a “J” region of about 12 or more amino acids, with the heavy chain also including a “D” region of about 10 or more amino acids. See generally, Fundamental Immunology, Paul, W., ed., 2nd ed. Raven Press, N.Y., 1989, Ch. 7 (incorporated by reference in its entirety for all purposes).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FRI, CDR1, FR2, CDR2, FR3, CDR3, and FR4. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3. When the application discloses a VL sequence with R as the C-terminal residue, the R can alternatively be considered as being the N-terminal residue of the light chain constant region. Thus, the application should also be understood as disclosing the VL sequence without the C-terminal R.

The term “antibody” includes intact antibodies and binding fragments thereof. Typically, fragments compete with the intact antibody from which they were derived for specific binding to the target including separate heavy chains, light chains Fab, Fab′, F(ab′).sub.2, F (ab) c, Dabs, nanobodies, and Fv. Fragments can be produced by recombinant DNA techniques, or by enzymatic or chemical separation of intact immunoglobulins. The term “antibody” also includes a bispecific antibody and/or a humanized antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)).

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996). Epitopes may be found in antibodies themselves, and may also comprise non-protein sites that are not formed from amino acids but from other constituents found in the body such as lipids, sugars, fats, and nucleic acids. Examples phosphorylcholine (PC) and phosphatidylcholine (PtC).

Antibodies, receptors, ligands and other proteins, protein fragments or nucleoside sequences that recognize the same or overlapping epitopes can be identified in a simple immunoassay showing the ability of one antibody, receptor, ligand and other protein, protein fragment or nucleoside sequence to compete with the binding of another antibody, receptor, ligand and other protein, protein fragment or nucleoside sequence to a target antigen. The epitope of an antibody, receptor, ligand and other protein, protein fragment or nucleoside sequence can also be defined by X-ray crystallography and nuclear magnetic resonance (NMR) when the epitope(s) of the antigen is (are) bound to the antibody, receptor, ligand and other protein, protein fragment or nucleoside sequence to identify contact residues. Alternatively, two antibodies, receptor, ligand and other protein, protein fragment or nucleoside sequence have the same epitope if all amino acid mutations in the antigen that reduce or eliminate binding of one antibody reduce or eliminate binding of the other. Two antibodies, receptors, ligands and other proteins, protein fragments or nucleoside sequences have overlapping epitopes if some amino acid mutations that reduce or eliminate binding of one antibody receptor, ligand and other protein, protein fragment or nucleoside sequence reduce or eliminate binding of the other.

The term “biomarker” as used herein refers to an indicator, e.g., a predictive, diagnostic, and/or prognostic indicator, which can be detected in a sample. The biomarker may serve as an indicator of a particular subtype of a disease or disorder (e.g., cancer) characterized by certain, molecular, pathological, histological, and/or clinical features. In some embodiments, the biomarker is a gene. In some embodiments, the biomarker is a variation (e.g., mutation and/or polymorphism) of a gene. In some embodiments, the biomarker is a translocation. Biomarkers include, but are not limited to, polynucleotides (e.g., DNA, and/or RNA), polypeptides, polypeptide and polynucleotide modifications (e.g., posttranslational modifications), proteins, carbohydrates, and/or lipid and glycolipid-based molecular markers.

The “presence,” “amount,” or “level” of a biomarker associated with an increased clinical benefit to an individual is a detectable level in a sample. These can be measured by methods known to one skilled in the art and also disclosed herein. The expression level or amount of biomarker assessed can be used to determine the response to the treatment.

The term “diagnosis” is used herein to refer to the identification or classification of a molecular or pathological state, disease or condition (e.g., an inflammatory disease, for example, inflammatory bowel disease). For example, “diagnosis” may refer to identification of a particular type of neurodegenerative proteinopathy disease, for example, Alzheimer's disease. “Diagnosis” may also refer to the classification of a particular subtype of disease, e.g., by histopathological criteria, or by molecular features (e.g., a subtype characterized by expression of one or a combination of biomarkers (e.g., particular genes or proteins encoded by said genes)).

The phrase “substantially similar,” as used herein, refers to a sufficiently high degree of similarity between two numeric values (generally one associated with a molecule and the other associated with a reference/comparator molecule) such that one of skill in the art would consider the difference between the two values to not be of statistical significance within the context of the biological characteristic measured by said values (e.g., protein disaggregation values). The difference between said two values may be, for example, less than about 20%, less than about 10%, and/or less than about 5% as a function of the reference/comparator value.

The phrase “substantially different,” refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., protein disaggregation values). The difference between said two values may be, for example, greater than about 10%, greater than about 20%, greater than about 30%, greater than about 40%, and/or greater than about 50% as a function of the value for the reference/comparator molecule.

As used herein, the term “proteinopathy” or “proteinopathic” refers to a disease, disorder, and/or condition associated with the pathogenic or pathologic aggregation and/or accumulation of one or more types of proteins, for example, but not limited to α-synuclein, β-amyloid, and/or tau proteins. In some embodiments, a proteinopathy is characterized by an anomaly in one or more of protein production, folding, aggregation, metabolism, disposal or degradation (e.g., autophagy), transportation, etc. In some embodiments, proteinopathies are neurodegenerative diseases. In some embodiments, proteinopathies are inflammatory diseases. In some embodiments, proteinopathies are cardiovascular diseases. In some embodiments, proteinopathies are proliferative diseases. Specific pathologies such as synucleinopathies, tauopathies, amyloidopathies, TDP-43 proteinopathies and others are examples of proteinopathies. Exemplary proteins implicated in proteinopathies include: α-synuclein in the case of Parkinson's disease, Lewy body disease, and other synucleinopathies; tau and β-amyloid in the case of Alzheimer's disease and certain other neurodegenerative diseases; SOD1 and TDP-43 in the case of amyotrophic lateral sclerosis; huntingtin in the case of Huntington's disease; rhodopsin in the case of retinitis pigmentosa.

An “effective amount” of an agent, e.g., a pharmaceutical formulation, refers to an amount effective, at dosages and for periods of time necessary, to achieve the desired therapeutic or prophylactic result.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical formulation, other than an active ingredient, which is nontoxic to a subject. A pharmaceutically acceptable excipient includes, but is not limited to, a buffer, a carrier, a diluent, a stabilizer, or a preservative.

As used herein, the term “sample” refers to a biological sample or pharmacological preparation obtained or derived from a source of interest, or a pharmacologic product, for example, a vial of liquid solution containing a biotherapeutic, for example, insulin, an antibody or an antibody fragment, enzymes, recombinant protein products, viruses, and the like as described herein. In some embodiments, a source of interest comprises an organism, such as an animal or human. In some embodiments, a biological sample comprises biological tissue or fluid derived from fluids found in the body of a subject, or within a body cavity of a subject, such as peripheral blood, plasma, umbilical cord blood, urine, stool, saliva, sputum, colostrum, breast milk, bone marrow, lymph fluid, cerebral spinal fluid, peritoneal fluid, pleural fluid, joint fluid, vitreous fluid, and inflammatory fluid. In some embodiments, a biological sample is or comprises cellular elements within the fluids and tissues described above, ascites; tissue or fine needle biopsy samples; free floating nucleic acids; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, centrifugation and/or filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

A “pharmacological product” (also referred to herein as a “pharmacologic product”, a “pharmacologic preparation”, a “pharmacological preparation”, a “medicament”, “therapeutic composition”, “pharmaceutical composition” or a “medicinal product”) is a term that broadly encompasses liquid solutions containing a peptide, a protein, a microorganism, for example, a bacteria, virus or yeast, or cells for use in the treatment of a disease or condition of a subject. In most illustrative examples, pharmacological products covers solutions containing biotherapeutic agents, recombinant products, and proteinacious containing solutions used in the preparation of medicinal products, for example, human serum albumin used in the preparation of vaccines and other medicaments.

Susceptible to: An individual who is “susceptible to” a disease, disorder, and/or condition (e.g., any disease, disorder, and/or condition, including, but not limited to, any disease, disorder, and/or condition described herein) is at risk for developing the disease, disorder, and/or condition. In some embodiments, an individual who is susceptible to a disease, disorder, and/or condition does not display any symptoms of the disease, disorder, and/or condition. In some embodiments, an individual who is susceptible to a disease, disorder, and/or condition has not been diagnosed with the disease, disorder, and/or condition. In some embodiments, an individual who is susceptible to a disease, disorder, and/or condition is an individual who has been exposed to conditions associated with development of the disease, disorder, and/or condition (e.g., the individual has been exposed to an infectious agent; the individual has been exposed to an environmental hazard thought to cause the disease, disorder, and/or condition; etc.). In some embodiments, a risk of developing a disease, disorder, and/or condition is a population-based risk (e.g., an individual carries a gene and/or allele associated with the disease, disorder, and/or condition).

Synucleinopathy: As used herein, the term “synucleinopathy” or α-synucleinopathy” refers to diseases, disorders, and/or conditions that are associated with or characterized by pathological accumulation of the protein α-synuclein, including but not limited to Parkinson's disease, Lewy body disease, multiple system atrophy, Hallervorden-Spatz disease, and frontotemporal dementia.

Tauopathy: As used herein, the term “tauopathy” or “tauopathic” refers to diseases, disorders, and/or conditions that are associated with or characterized by pathological accumulation of the tau protein, including but not limited to Alzheimer's disease, frontotemporal dementia, and progressive supranuclear palsy.

Therapeutic Agent: As used herein, the phrase “therapeutic agent” refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is any substance that can be used to reverse, alleviate, ameliorate, relieve, inhibit, delay onset of, reduce severity of, and/or reduce incidence of one or more symptoms or features of a disease, disorder, and/or condition.

The terms “subject” and “individual” and “patient” are used interchangeably herein, and refer to an animal, for example a mammal, for example, a human or non-human mammal, to whom treatment, including prophylactic treatment, with a pharmaceutical composition as disclosed herein, is provided. The term “subject” as used herein refers to human and non-human animals. The term “non-human animals” includes all vertebrates, e.g., mammals, such as non-human primates, (particularly higher primates and monkeys), horses, donkeys, sheep, dogs, rodents (e.g., mouse, hamster, chipmunk or rat), guinea pigs, lambs, goats, pigs, cattle, bison, buffalo, cats, birds, rabbits, cows, wolves, deer, and non-mammals such as chickens, fish, molluscs, crustaceans, snakes, frogs, amphibians, reptiles, cephalopods (eg, squid), etc. In one embodiment, the subject is human. In another embodiment, the subject is an experimental animal or animal substitute as a disease model. Non-human mammals include mammals such as non-human primates, (particularly higher primates and monkeys), horses, sheep, dogs, rodents (e.g., mouse, hamster, chipmunk or rat), guinea pigs, lambs, goats, pigs, cattle, bison, buffalo, cats, birds, rabbits, cows, wolves, and deer. In some aspects, the non-human animal is a companion animal such as a dog or a cat.

As used herein, “treatment” (and variations such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, reversal and/or alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the disclosure (e.g., antibodies targeting one or more of the proteins or other agents discussed herein) are used to delay development of a disease or to slow the progression of a disease, or to prevent, delay or inhibit the development of a side effect related to the treatment of a different disease being actively treated.

As is understood by one skilled in the art, reference to “about” a value or parameter herein includes (and describes) embodiments that are directed to that value or parameter per se or that have a variance plus or minus of that value ranging from less than 10%, or less than 9%, or less than 8%, or less 7%, or less than 6%, or less than 5%, or less than 4%, or less than 3%, or less than 2%, or less than 1%, or less than 0.1% than the stated value. For example, description referring to “about X” includes description of “X”.

It is understood that aspect and embodiments of the disclosure described herein include “consisting” and/or “consisting essentially” of aspects and embodiments. As used herein, the singular form “a”, “an”, and “the” includes plural references unless indicated otherwise.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One illustrative example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

The term “derivative” as used herein refers to peptides which have been chemically modified, for example but not limited to by techniques such as ubiquitination, labeling, pegylation (derivatization with polyethylene glycol), lipidation, glycosylation, or addition of other molecules. A molecule may also also a “derivative” of another molecule when it contains additional chemical moieties not normally a part of the molecule. Such moieties can improve the molecule's solubility, absorption, biological half-life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., Mack Publ., Easton, Pa. (1990), incorporated herein, by reference, in its entirety.

The term “functional” when used in conjunction with “fragment” “mimetic”, “derivative” or “variant” refers to a protein or polypeptide of the disclosure which possesses a biological activity (either functional or structural) that is substantially similar to a biological activity of the entity or molecule it is a functional derivative or functional variant thereof, i.e., a protein or polypeptide that disaggregates protein complexes into either smaller complexes or soluble fragments of complexes, for example, wherein said protein complex disaggregation provides some therapeutic benefit and/or that the smaller complexes or soluble fragments of complexes do not cause or exacerbate the conditions, symptoms or pathology of the disease being treated.

The articles “a” and “an” are used herein to refer to one or to more than one (i.e., at least one) of the grammatical object of the article. By way of example, “an element” means one element or more than one element. Other than in the operating examples, or where otherwise indicated, all numbers expressing quantities of ingredients or reaction conditions used herein should be understood as modified in all instances by the term “about.”

It should be understood that this disclosure is not limited to the particular methodology, protocols, and reagents, etc., described herein and as such can vary. The terminology used herein is for the purpose of describing particular embodiments only and is not intended to limit the scope of the present disclosure, which is defined solely by the claims. Other features and advantages of the disclosure will be apparent from the following Detailed Description, the drawings, and the claims.

The present disclosure is not to be limited in scope by the specific examples described herein, which are intended for the purpose of exemplification only. Functionally-equivalent compositions and methods are clearly within the scope of the disclosure.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, formulation and medical treatments in neurology, cardiology, hematology, oncology and other areas of medical intervention in human illness and disease. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); Textbook of Interventional Cardiology, 7th Edition, Authors: Eric J. Topol & Paul S. Teirstein; and Animal Cell Culture: Practical Approach, Third Edition (John R. W. Masters, ed., 2000), ISBN 0199637970, whole of text; each of these references are incorporated herein by reference in their entireties. Protein aggregates, also known as protein multimers and/or oligomers, and/or fibrils, and as dysfunctional or disordered protein deposits, are known to be associated pathogenically with neurodegenerative and other diseases, for example, Alzheimer's Disease, Parkinsons's Disease, Hungtington's Disease, ALS, Spongiform encephalopathies, multiple Tauopathies, cataracts, amyloid transthyretin cardiomyopathy, type-2 diabetes, primary and secondary systemic amyloidosis, some forms of atherosclerosis, hemodialysis-related disorders, and short-chain amyloidosis, among many others (see also par. 0002). Accurate measurement of protein aggregates during in vitro work and with clinical and pharmaceutical samples is imperative. However, current methods are deficient in filling this need, because current methods fail to readily provide a measure of protein aggregate size. The present disclosure now provides a significant advance to the field by developing a new microparticle immunocapture assay that provides, at the same time, measurement of protein aggregate amount and size. In addition, the assay is simple, specific, quantitative, and quick, providing results within a single experimental day. The assays described herein are able to discriminate between protein monomers that exist in vivo that do not cause cellular damage from those same monomers that become oligomers and multimers and develop into pathological protein aggregates that cause cellular and tissue damage. In addition, the assays of the present disclosure cannot only quantitatively identify the presence of protein aggregates, but also quantitatively provide amounts of protein aggregates in unknown test samples. For example, the assays of the present disclosure provide a means to evaluate disease-associated protein aggregates in clinical settings (eg, Alzheimer's Disease, Parkinson's Disease, Huntington's Chorea, Amyotrophic Laterial Sclerosis (ALS) traumatic brain injury (TBI), and prion propagated diseases) taken from cerebral spinal fluid, peripheral blood, and and other fluid and tissue preparation test samples.

Cellular stress and toxicity are often associated with the formation of protein multimers, or aggregates. Numerous degenerative disorders, including Alzheimer's, Parkinson's and Huntington's disease, prion propagated disease, amyotrophic lateral sclerosis, cardiac amyloidosis, and diabetes and other illnesses are characterized by aggregated protein deposits. Presently available methods fail to adequately assess multimer size along with multimer quantitation, and typically fail to incorporate one or more ancillary traits including target specificity, operative simplicity, process speed, and reliance on readily available instrumentation. The present disclosure provides a microparticle immunocapture assay that combines the advantages inherent to a monoclonal antibody: protein interaction with highly quantitative flow cytometry analysis. The presently described assays and uses thereof use established reagents to build the assay and demonstrate that the described assay embodiments are highly adaptable to measure multimer (aggregate) size and quantity at the same time in a specific, simple, rapid and technically streamlined workflow.

The present disclosure provides a microparticle immunocapture assay that discriminates between monomer and protein multimers (aggregates). The failure of proteins to fold into correct three-dimensional structures can lead to diseases called proteopathies (sometimes also referred to as protein-aggregation diseases, protein misfolding diseases, proteinopathies or protein conformational disorders). The failure may be due to one or more mutations in the proteins' gene or to environmental factors such as oxidative stress, alkalosis, acidosis, pH shift and osmotic shock. The misfolding of proteins can sometimes lead to clumping or aggregation into amyloid plaques or fibrils that can exacerbate a disease. Proteopathies cover a wide spectrum of afflictions, including neurodegenerative diseases (e.g., Alzheimer's, Parkinson's, polyglutamine diseases such as Huntingtin in Huntington's disease, prion diseases); amyloidosis of other non-nervous system proteins such as 1-antitrypsin, immunoglobulin light and heavy chains, lactadherin, apolipoprotein, gelsolin, lysozyme, fibrinogen, atrial natriuretic factor, keratin, lactoferrin and beta-2 microglobulin, among others); sickle cell disease; cataracts; cystic fibrosis; retinitis pigmentosa; and nephrogenic diabetes insipidus.

Amyloidosis refers to the pathological deposition of proteins in the form of congophilic, green birefringent fibrils, when congo red-stained, either dispersed or in the form of localized amyloidomas. Such deposits are symptomatic of several diseases, for example Alzheimer's Disease, inflammation-associated amyloid, type II diabetes, bovine spongiform encephalopathy (BSE), Creutzfeld-Jakob disease (CJD), scrapie and primary amyloidosis.

Amyloidoses are generally categorized into three groups: major systemic amyloidoses, major localized amyloidoses, and miscellaneous amyloidoses. Major systemic amyloidoses include: chronic inflammatory conditions (e.g., tuberculosis, osteomyelitis, etc.); non-infectious conditions such as juvenile rheumatoid arthritis, ankylosing spondylitis and Crohn's disease, etc.; familial Mediterranean Fever, plasma cell dyscrasia (primary amyloidosis) and various familial polyneuropathies and cardiomyopathies. Major localized amyloidoses include: dialysis-related amyloidosis, Alzheimer's disease, Down syndrome, Hereditary cerebral hemorrhage (Dutch), and non-traumatic cerebral hemorrhage of the elderly. Miscellaneous amyloidoses include: familial polyneuropathy (Iowa), familial amyloidosis (Finnish), hereditary cerebral hemorrhage (Icelandic), CJD, Medullary carcinoma of the thyroid, atrial amyloid, and diabetes mellitus (insulinomas). Other amyloidoses include those referenced in Louis W. Heck, “The Amyloid Diseases” in Cecil's Textbook of Medicine 1504-6 (W.B. Saunders & Co., Philadelphia, Pa.; 1996).

Transmissible spongiform encephalopathies which cause CJD and Gerstmann-Strassler-Scheinker (GSS) disease are described by B. Chesebro et al., “Transmissible Spongiform Encephalopathies: A Brief Introduction” in FIELD'S VIROLOGY 2845-49 (3rd Edition; Raven Publishers, Philadelphia, Pa.; 1996) and in D. C. Gajdusek, “Infectious amyloids: Subacute Spongiform Encephalopathies as Transmissible Cerebral Amyloidoses,” 2851-2900 in FIELDS VIROLOGY (1996). Many of these diseases are likely mediated by prions, an infectious protein. See S. B. Prusineri, “Prions” in FIELDS VIROLOGY 2901-50 (1996) and the references contained therein.

The methods of the present disclosure (as well as the compositions, systems and kits described below) find use in a variety of applications, including, e.g., research applications, clinical applications (e.g., clinical diagnostic applications), and pharmacologic applications etc.

### Compositions, Assays, and Kits

In various embodiments, the present disclosure provides novel microparticle-based immunocapture assays for quantitating aggregation of proteins, especially those that are involved in pathogenic protein aggregation (Proteinopathies). In some embodiments, the method for analysing a sample for the presence of an aggregated protein includes:
a. providing a capture substrate comprising a microparticle conjugated to a first capture moiety;
b. blocking the capture substrate non-specific binding sites;
c. incubating the capture substrate with a test sample suspected of having an aggregated protein for a period of time sufficient for the aggregated protein to specifically bind to the first capture moiety; wherein the first capture moiety binds specifically to the aggregate protein if present, thereby forming a capture complex on the capture substrate,
d. incubating the capture substrate with a second capture moiety that specifically binds to the aggregated protein, wherein the second capture moiety is coupled to a signalling moiety, and wherein the signaling moiety comprises a detectable label; and
e. determining the amount of detectable label present on the surface of the capture substrate.

In various embodiments, the microparticle based assay is a microparticle-based immunocapture assay, for example, a bead-based immunocapture assay, wherein the plurality of microparticles used to quantitatively and qualitatively measure the presence of an aggregated protein are a plurality of microparticle beads. Incubating the various reagents and components used in the assay generally refer to incubation of the products for a time sufficient to perform the recited step, for example, 5 minutes to 360 minutes, optionally, with a completion of at least 20%-50%. For example, the step (c) incubating the capture substrate with a test sample suspected of having an aggregated protein for a period of time sufficient for the aggregated protein to specifically bind to the first capture moiety, may refer to a period of time, for example 5 minutes to 360 minutes, or more, optionally, in which at least 20%-50% of the first capture moiety present on the has bound to aggregated protein, thereby forming a capture complex.

### Microparticles

In various embodiments, microparticles forming the foundational substrate for performing the described assays are microparticles having as its greatest dimension, less than 100 μm in size, for example, less than 50 μm, or less than 25 μm, or less than 15 μm, or less than 10 μm, or less than 5 μm, or less than 1 μm, or less than 0.1 μm, or less than 0.01 μm in size, for example, ranging from about 50 μm, to about 0.01 μm, or from about 25 μm, to about 0.1 μm, or from about 10 μm, to about 0.1 μm. In certain preferred embodiments, the microparticles have as its greatest dimension, a size ranging from 0.1 μm to about 10 μm in size. In some embodiments, the greatest dimension of the microparticle ranges from 0.001 μm to 1000 μm, from 0.5 μm to 100 μm, from 0.1 μm to 20 μm, 20 μm or less, 15 μm or less, 10 μm or less, 5 μm or less, 1 μm or less, 0.75 μm or less, 0.5 μm or less, 0.4 μm or less, 0.3 μm or less, 0.2 μm or less, 0.1 μm or less, 0.01 μm or less, or 0.001 μm or less.

The microparticles may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In each of these various shapes, the beads may be solid, or partially solid, or they may also be hollow microparticles, for example, hollow beads.

The microparticles may be made of any suitable material, including but not limited to, glass and modified or functionalized glass, plastics (e.g., acrylics, polystyrene and copolymers of styrene and other materials, polypropylene, polyethylene, polybutylene, polyurethanes, TEFLON®, and the like), polysaccharides, nylon or nitrocellulose, composite materials, ceramics, and plastic resins, silica or silica-based materials including silicon and modified silicon, carbon, for example, carbon fiber beads, metals, inorganic glasses, plastics, optical fiber bundles, and a variety of other polymers. In general, the substrates allow optical detection and do not appreciably fluoresce. In various embodiments, the microparticles are functionalized glass or plastic beads, or paramagnetic beads.

In various embodiments, the microparticles of the present disclosure can include bead microparticles. The plurality of beads can be spherical, including semi-spherical, in shape. In other embodiments, the plurality of beads can be ovoid in shape. In some embodiments, the plurality of beads can be cubical in shape including, but not limited to, rectified cubes, rectangular cubes, truncated cubes, cantellated cubes, omnitruncated cubes, or snub cubes. In some embodiments, the plurality of beads can be cylindrical in shape including, but not limited to, right circular cylinders, elliptic cylinders, or oblique cylinders. In some embodiments, the plurality of beads can be conic in shape including, but not limited to, right circular cones or oblique circular cones. In some embodiments, the plurality of beads can be pyramidal in shape including, but not limited to, square pyramids or pentagonal pyramids. In some embodiments, the plurality of beads can be tetrahedral in shape. In some embodiments, the plurality of beads can be prismic in shape. Additionally, other embodiments of the plurality of beads can be any form of polyhedron including, but not limited to, dodecahedrons, icosidodecahedrons, rhombic triacontahedrons, or rhombic dodecahedrons.

In various embodiments, the microparticles of the present disclosure have been functionalized to permit covalent coupling to a first capture moiety, for example, an antibody, or an antigen binding fragment thereof that specifically binds to an aggregate protein. As will be appreciated by those in the art, this will be done depending on the composition of the immobilized first capture moiety and the microparticle surface material (e.g., beads). The functionalization of solid support surfaces such as certain polymers with chemically reactive groups such as thiols, amines, carboxyls, and the like is generally known in the art. Accordingly, “blank” microparticles (e.g., beads) may be used that have surface chemistries that facilitate the attachment of the desired functionality by the user. In certain embodiments, first capture moieties, for example, antibodies and antigen-binding fragments thereof can be covalently attached to microparticles (e.g., beads) using any suitable chemical reaction, e.g., cycloaddition (e.g., an azide-alkyne Huisgen cycloaddition (e.g., a copper (I)-catalyzed azide-alkyne cycloaddition (CuAAC) or a strain-promoted azide-alkyne cycloaddition (SPAAC))), amide or thioamide bond formation, a pericyclic reaction, a Diels-Alder reaction, sulfonamide bond formation, alcohol or phenol alkylation, a condensation reaction, disulfide bond formation, or a nucleophilic substitution.

In some instances, a composition described herein (e.g., a first capture moiety and/or microparticle) may include a capture conjugating moiety. A capture conjugating moiety may include at least one functional group that is capable of undergoing a conjugation reaction, for example, any conjugation reaction described in the preceding paragraph. The conjugation moiety can include, without limitation, a 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a thiol, or a thioisocyanate group that permits functional coupling of the first capture moiety to the surface of a microparticle (e.g. beads).

In some embodiments, the assays, compositions and kits of the present disclosure comprises a plurality of beads as the microparticle. The beads are reactive in the sense that they contain reactive chemical groups that permit coupling to the first capture moiety, for example, an antibody or an antigen-binding fragment thereof. The beads may be colorless and/or transparent or may be opaque, provided that the beads do not appreciably fluoresce when exposed to wavelengths of light that fluoresce the detectable label, for example a fluorophore. The beads may be non-magnetic or paramagnetic to facilitate processing and handling when incubating with various reagents. In various embodiments, the beads are made of glass and modified or functionalized glass, plastics (e.g., acrylics, polystyrene and copolymers of styrene and other materials, polypropylene, polyethylene, polybutylene, polyurethanes, TEFLON®, and the like) and other plastic polymers that do not appreciably fluoresce. In various embodiments, the beads range in size (as measured by its longest dimension) ranging from about 0.01 μm to about 100 μm, for example, from about 0.5 μm to about 50 μm, and more preferably from about 0.5 μm to about 10 μm.

In some embodiments, the microparticles can include, but not limited to microparticles that are a sphere, bead, pellet, or non-planar shape composed of one or more of the following components: glass and modified or functionalized glass (e.g., carboxymethyldextran functionalized glass), plastics (including acrylics, polystyrene and copolymers of styrene and other materials, polypropylene, polyethylene, polybutylene, polyurethanes, Teflon®, polysaccharides, nylon, nitrocellulose, composite materials, ceramics, plastic resins, silica or silica-based materials including silicon and modified silicon (e.g., patterned silicon), carbon, metals, quartz (e.g., patterned quartz), inorganic glasses, plastics, optical fiber bundles, and other polymers. The microparticle can be composed of latex, polystyrene, silica, a magnetic material, a paramagnetic material, or any combination thereof. For example, the microparticle may be spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. Similarly, any of the microparticles disclosed above may include a polymer bead, a solid core bead, a carbon fiber bead, a hollow bead, a paramagnetic bead, or a microbead.

### First Capture Moieties

In various embodiments, the first capture moiety serves to specifically bind to an aggregated protein that is the object of the assay. Aggregate proteins discussed herein are implicated in one or more neurodegenerative and other non-neurodegenerative diseases and cause harm due to their misfolded state. As such, the first capture moiety is conjugated to the surface of the microparticle and is also oriented such that the binding structures of the first capture moiety specifically binds to the aggregated protein in the assay. The first capture moiety may be conjugated to the microparticle in any number of ways known to those skilled in the art. For example, in some non-limiting examples, the first capture moiety may be conjugated to a surface of the microparticle, wherein the microparticle is at least partially coated with a reactive moiety comprising an amino, a carboxyl, a thiol, or a hydroxyl reactive moiety that facilitates conjugation with the first capture moiety. In some embodiments, the microparticle allows optical detection and does not appreciably fluoresce. In related embodiments, the microparticle is a paramagnetic bead. In some non-limiting embodiments, the microparticle is a latex bead coated with aldehyde sulfate reactive moiety.

While not wishing to be bound by any specific theory, the first capture moiety can bind to peptides, epitopes, single or cross-related antigens, or a selection of amino acids or epitopes present on the aggregated protein or non-protein molecule. The epitope or antigen selected is preferably not an epitope or antigen that is found in other proteins and is specific for the aggregated protein, or aggregated moieties and other molecules being investigated in the assay.

In several embodiments of the present disclosure, the first capture moiety can be an antibody, or antigen binding fragment thereof. In other exemplary embodiments, the first capture moiety can be non-antibody moieties that specifically bind to peptides, epitopes, single or cross-related antigens, or a selection of amino acids present on the aggregated protein, for example, non-antibody capture moieties such as receptors, ligands, aptamers, DNA (oligonucleotides or other polynucleotides of greater length than 20-50 nucleotides, or double stranded DNA segments), RNA and lipids. In all circumstances, the first capture moiety specifically binds to one or more aggregated proteins, preferably a single aggregated protein, or other aggregated molecule.

In various embodiments, an average number of first capture moieties to microparticle varies from capture moieties to capture moieties. In some embodiments, the average number of first capture moieties per microparticle is one in which the microparticle is not saturated in the coating of the first capture moiety. In some illustrative embodiments, 1 μL of microparticles ranging in size from about 0.01 μm to about 100 μm are coated with 0.001 μg to about 100 μg of the first capture moiety.

In various embodiments, the first capture moiety is an antibody, or antigen-binding fragment thereof that specifically binds to an epitope or antigen of an aggregated protein. In various embodiments, the first capture moiety is a human or mouse antibody that binds to a single epitope and/or antigen. In related embodiments, the first capture moiety is a monoclonal antibody. In another embodiment, the first capture moiety is a monospecific human or humanized antibody from any subclass, e.g., IgG that binds to a single epitope and/or antigen, or receptor, ligand, aptamer or nucleoside sequence. In various embodiments, the first capture moiety specifically binds to an aggregated protein selected from native, variant, mutant or posttranslationally modified forms of α-synuclein, tau, amyloid beta (Aβ42 and Aβ40), SOD1, TDP-43, FUS, huntingtin, transthyretin, prion proteins, crystallin, immunoglobulin light chain, serum amyloid A, beta2-microglobulin, lysozyme, gelsolin, calcitonin, prolactin, IAPP or amylin, fibrinogen, rhodopsin, glucosylceramide, hemoglobin, DNA binding proteins, RNA binding proteins and other proteins with a potential for aggregation that have been implicated, or may be suspected of involvement in, other diseases, and other proteins studied in the laboratory setting and other proteins contained in pharmacologic preparations for which protein aggregation is assessed that include preparations of antibody and antigen binding fragments against CD3, against CD4, against CD11, against CD19, against CD20, against CD22, against CD30, against CD38, against CD52, against CD79b, against PD-1, against PD-L1, against PD-L2, against CCR4, against IL-1, against IL-4R, against IL-5, against IL-5R, against IL-6, against IL-6 receptor, against IL-8, against IL-13, against IL-17, against IL-17 receptor, against IL-23, against IL-33, against IL-36 receptor, against HER2, against tissue factor, against CCR4, against EGFR, agasinst PDGRFa, against IFNAR1, against sclerostin, against von Willebrand factor, against C5, against IFNgamma, against FGF23, against Factor IXa, against Kalikrein, against complement C5, against BCMA, against angiopoietin-like 3, against TROP-2, against IGF-1R, against CGRP, SLAMF7, against PCSKg, against GD2, against Nectin-4, against P-selectin, against Ebola virus, against IgE, against GD2, against BLyS, against RANK-L, against B7-H3, against MASP-2, against LAG-3, against VEGF, against alpha4beta7 integrin, against C1s, against thymic stromal lymphopoietin, against folate receptor alpha, against RSV, against CTLA-4, against FcRn, against GPIIb/IIIa, against Ep/cAM, against endotoxin, against TNF, against G protein-coupled receptor 5D, against the COVID-19 spike protein and variants thereof, against Clostridium difficile enterotoxin B, plus protein preparations of IL-2, IL-7, IL-8, IL-10, IL-12, IL-15, IL-18, IL-23, and IL-36 proteins and/or other proteins or molecules that form oligomers, multimers, fibrils and/or deposits evident in the diseases described herein, and also in the pharmacological products described herein.

First capture antibodies may be commercially available as antibody specific reagents that bind to aggregated proteins involved in Proteinopathies, and proteins used in the manufacture of pharmacological products, for example, biotherapeutics as exemplified herein. In some embodiments, known antibodies useful as first capture moieties may include, for example, those produced by Neurimmune AG, Zurich, Switzerland (Aducanumab (for Alzheimer's Disease, Neurimmune), NI005/AP-101 (for ALS, Neurimmune), BIIB076 (for Alzheimer's Disease, Neuimmune), NI004 (for Alzheimer's Disease, Neurimmune), NI006 (for Transthyretin amyloid (ATTR)-cardiomyopathy), NI308 (for Frontotemporal Dementia/ALS, Neuimmune), NI302 (for Huntingtin, Neurimmune), NI205 (for Frontotemporal dementia and ALS), NI504 (for neurodegeneration, Neurimmune), AAV hmAb (for taupathies, Neurimmune) and AAV hmAb (for synucleinopathies, Neurimmune). Other exemplary primary capture moieties may include any of the following monoclonal antibodies:

TABLE 1
Examples of first capture moieties
from Proteintech (Rosemont IL, USA)

| | Catalog | | |
|---|---|---|---|
| Antibody Name | Number | Type | Applications |
| C9orf72 | 66140-1-Ig | Mouse | ELISA, IHC, WB |
| | | Mono | |
| CREST (SS18L1) | 60314-1-Ig | Mouse | ELISA, WB |
| | | Mono | |
| FUS/TLS | 60160-1-Ig | Mouse | ELISA, IF, IHC, WB |
| | | Mono | |
| OPTN | 60293-1-Ig | Mouse | ELISA, WB |
| | | Mono | |
| Phospho- | 66079-1-Ig | Mouse | ELISA, WB |
| TDP-43 (403/404) | | Mono | |
| TDP-43 | 60019-2-Ig | Mouse | ELISA, IF, IHC, WB |
| (Human specific) | | Mono | |
| TMEM106B | 60333-1-Ig | Mouse | ELISA, WB |
| | | Mono | |
| VAPB (ALS8) | 66191-1-Ig | Mouse | ELISA, IF, IHC, WB |
| | | Mono | |
| VCP | 60316-1-Ig | Mouse | ELISA, IHC, WB |
| | | Mono | |
| Amyloid beta (A4) | 60342-1-Ig | Mouse | ELISA, IHC, WB |
| precursor protein | | Mono | |
| EPHA1-specific | 60155-2-Ig | Mouse | ELISA, IHC, WB |
| | | Mono | |
| GFAP 6 | 60190-1-Ig | Mouse | ELISA, IHC, IP, WB |
| | | Mono | |
| GLUT1 | 66290-1-Ig | Mouse | ELISA, FC, IHC, WB |
| | | Mono | |
| Glutamine synthetase | 66323-1-Ig | Mouse | ELISA, IHC, WB |
| | | Mono | |
| GRP94 | 60012-1-Ig | Mouse | ELISA, FC, IF, WB |
| | | Mono | |
| NR1H3 | 60134-1-Ig | Mouse | ELISA, IF, WB |
| | | Mono | |
| FUS/TLS | 60160-1-Ig | Mouse | ELISA, IF, IHC, IP, WB |
| | | Mono | |

In the table above, the term “mouse mono” refers to “mouse monoclonal antibody (mAb)

Other examples of first capture moieties, include monoclonal antibodies from Thermo Fisher Scientific, for example: Alpha synuclein, clone Syn211, cat #32-8100, TDP-43, clone JM51-10, cat #MA5-32627, and Tau, clone HT7, cat #MN1000.

In some illustrative embodiments, the first capture moiety is a polynucleotide sequence that is known to bind to DNA binding or RNA binding proteins. In various embodiments, the first capture moiety can be a sequence of DNA, single stranded or double stranded and can range from about 5 to about 100 deoxyribonucleotides. In related embodiments, the first capture moiety can be a sequence of RNA, single stranded, and can range from about 5 to about 100 ribonucleotides.

In various embodiments, the DNA and/or RNA can be modified to prevent cleavage from nucleases by inserting modified nucleobases or modified sugars that resist nuclease cleavage.

### Second Capture Moieties

In various embodiments, the assay, compositions and kits of the present disclosure employ the use of a second capture moiety which has the same specificity for the epitope and/or antigen, or amino acid sequence targeted by the first capture moiety. In various embodiments, the second capture moiety is the same capture moiety as the first capture moiety. For example, the second capture moiety is the same as the first capture moiety, and can include an antibody, or antigen binding fragment thereof, or a receptor or a ligand or a DNA or RNA polynucleotide sequence. In all circumstances, the first capture moiety and second capture moiety specifically binds to one or more aggregated proteins or other aggregated molecules. In some embodiments, the second capture moiety can be an antibody, or antigen-binding fragment thereof, or other molecules, for example, peptides, proteins, receptors, ligands, lipids, nucleic acid molecules (DNA and RNA), or aptamers, preferably that binds a single aggregated protein or other aggregated molecules, wherein the first capture moiety and the second capture moiety can be the same or different. In various embodiments, the second capture moiety can comprise a single type of molecule or the second capture moiety can be two or more different types of molecules that bind to the same one or more aggregated proteins (provided that there is a first capture moiety that binds to the same aggregated protein as the second capture moiety), and other molecules, for example, peptides, proteins, receptors, ligands, nucleic acids, or aptamers, preferably a single aggregated protein.

In some embodiments, a second component of the composition of the present invention comprises a second capture moiety. In some illustrative embodiments, the second capture moiety comprises an antibody or antigen binding fragment thereof, a nucleic acid molecule (DNA or RNA), a receptor, a ligand, and aptamer, a lipid which binds to the same epitope or antigen, or sequence of amino acids of an aggregated protein as the first capture moiety, wherein the second capture moiety is conjugated to a signalling moiety that comprises at least one of: a detectable label and a first binding partner that is operable to bind specifically to a detectable label that is coupled to a second binding partner

While the epitope and/or antigen binding portion of the second capture moiety is directed to the same epitope and/or antigen of the first capture moiety, the second capture moiety is coupled to a signalling moiety.

The second capture moiety comprises a signaling moiety. The signaling moiety comprises a detectable label or a first binding partner that is operable to bind to a detectable label that is conjugated to a second binding partner. For example, in some illustrative embodiments, the second capture moiety can be an antibody, or antigen binding fragment thereof, or a polynucleotide sequence, wherein the second capture moiety specifically binds to a DNA or RNA binding protein, or a protein receptor, ligand or aptamer, and each of these signaling moieties are conjugated, coupled, covalently or non-covalently attached to a detectable label, for example, a fluorescent label, a radiolabel, a luminescent agent, and a metal element label.

In some illustrative embodiments, the first capture moiety is a polynucleotide sequence that is known to bind to DNA binding or RNA binding proteins. In various embodiments, the first capture moiety can be a sequence of DNA, single stranded or double stranded and can range from about 5 to about 100 deoxyribonucleotides. In related embodiments, the first capture moiety can be a sequence of RNA, single stranded, and can range from about 5 to about 100 ribonucleotides.

In various embodiments, the DNA and/or RNA can be modified to prevent cleavage from nucleases by inserting modified nucleobases or modified sugars that resist nuclease cleavage.

In various embodiments, the signalling moiety can comprise a first binding partner selected from biotin, avidin or streptavidin as illustrative examples. In this particular set of examples, if the signalling moiety is a first binding partner, then the detectable label is conjugated, coupled, covalently or non-covalently attached to a second binding partner that is designed to specifically bind to the first binding partner of the second capture moiety. Illustrative examples of second binding partners include: biotin, avidin or streptavidin. For example, if the first binding partner coupled to the antibody, or antigen binding fragment thereof, or polynucleotide is biotin, then the second binding partner coupled to the detectable label may be avidin or streptavidin. If the first binding partner coupled to the antibody, or antigen binding fragment thereof, or polynucleotide is avidin or streptavidin, then the second binding partner coupled to the detectable label may be biotin.

While the above coupling reagents (first and second binding partners) are just examples of possible binding pairs, a person skilled in the art is also aware of other binding pairs that can be used as first and second binding partners, and the exemplified list is not an exhaustive set of examples of possible high affinity binding molecules, that can be employed to specifically provide a detectable label attached to a second binding partner when assaying a sample containing its corresponding binding signalling moiety.

### Signaling Moieties

In various illustrative embodiments, as described above, the assays, compositions and kits of the present disclosure employs a signalling moiety that is coupled to the second capture moiety. Exemplary signalling moieties can include a detectable label, for example, a fluorescent label, a radiolabel, a luminescent agent, and a metal element label. In other examples, a signaling moiety comprises a first binding partner selected from avidin/streptavidin, biotin and any other molecule that pairs or binds with high affinity or specifically, to a second binding partner that can be labeled with a detectable label.

### Detectable Labels

A “detectable label” in context of the present invention is a detectable marker that can produce a signal that is detectable by visual or instrumental means, e.g., an enzyme, or a fluorescent protein, a protein with the incorporation of a radiolabeled amino acid or attachment to a polypeptide of biotinyl moieties that can be detected by marked avidin or streptavidin (e.g., streptavidin containing a fluorescent marker or enzymatic activity that can be detected by optical or colorimetric methods). The detectable label can be a fluorescent label, a radiolabel, a luminescent agent, a metal element label, or an enzyme that can be detected with the aid of a suitable detection device, for example, a flow cytometer, a scintillation counter, a spectrophotometer, optical detectors and the like. In one embodiment, the detectable label is an enzymatic label. In such embodiments, a chromogenic, fluorogenic, or chemiluminescent enzyme substrate may be contacted with the enzyme to produce a detectable product (e.g., a signal). It is understood in the art that chromogenic, fluorogenic, or chemiluminescent enzyme substrates are known or can be made for many different enzymes. Thus, any known chromogenic, fluorogenic, or chemiluminescent enzyme substrate capable of producing a detectable product in a reaction with a particular enzyme can be used in the present disclosure.

In some embodiments, the detectable label can be a fluorescent label, a radiolabel, a luminescent agent, a metal element label, or an enzyme that can be detected with the aid of a suitable detection device, for example, a flow cytometer, a scintillation counter, a spectrophotometer, optical detectors and the like. In some embodiments, when the detectable label comprises an enzyme, a further agent that couples to the enzyme may be added to the assay, for example, a chromogenic, fluorogenic, or chemiluminescent enzyme substrate may be contacted with the enzyme to produce a detectable product (e.g., a signal). It is understood in the art that chromogenic, fluorogenic, or chemiluminescent enzyme substrates are known or can be made for many different enzymes. Thus, any known chromogenic, fluorogenic, or chemiluminescent enzyme substrate capable of producing a detectable product in a reaction with a particular enzyme can be used in the present invention. For example, in some embodiments in which the analyte is detected or quantified using a method as described herein in which the enzyme label is β-galactosidase, the enzyme substrate added to the array can be a β-galactosidase substrate such as resorufin-β-D-galactopyranoside or fluorescein di(β-d-galactopyranoside).

Examples of detectable labels coupled to the binding partner can include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm), chromogens, fluorescent labels (e.g., FITC, rhodamine, lanthanide phosphors), enzymatic labels (e.g., horseradish peroxidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), and magnetic agents (e.g., gadolinium chelates).

Representative examples of detectable labels commonly employed for immunoassays include moieties that produce light, e.g., acridinium compounds, and detectable labels that produce fluorescence, e.g., fluorescein. Other detectable labels can include chemilluminescent agents and substrates, which can be employed to provide chelmilluminescnece when appropriately excited with the correct wavelength.

Preferably the detectable label produces signals that are distinguishable, such as those labels that can produce fluorescence and chemilluminescence, for example, fluorescent proteins producing light signals with different wave lengths.

A fluorescent detectable label refers to a molecule that when excited with the necessary wavelength is able to fluoresce or produce light. For the purpose of the present invention a fluorescent protein or moiety is a protein that when excited with an appropriate wavelength results in emission of a light signal that may be detected. In a preferred embodiment the emission spectrum from the fluorescent protein according to the invention is between 445-660 nm, between 550-660 nm and most preferably between 550-660 nm.

Fluorescent and chemilluminescent proteins when used as detectable labels in context of the invention may be selected from known fluorescent or chemilluminescent molecules, for example, a fluorescent or chemilluminescent protein selected from: a green fluorescent protein selected from the group of EGFG, AcGFP, TurboGFP, Emerald, Azani Green and ZsGreen, b. blue fluorescent protein selected from the group of EBFP, Sapphire and T-Sapphire, c. cyan fluorescent protein selected from the group of ECFP, mCFP, Cerulean, CyPet, AmCyan1, Midori-Ishi Cyan and mTFPI (Teal), d. yellow fluorescent protein selected from the group of EYFP, Topaz, Venus, mCitrine, Ypet, PhiYFP, ZsYellow1 and mBanana, e. orange and red fluorescent proteins selected from Kusabira Orange, mOrange, dTomato, dTomato-Tandem, DsRed, DsRed2, DsRed-Express (T1), DSRed-Monomer, mTangerine, mStrawberry, AsRed2, mRFP1, Jred, mCherry, HcRed1, mRaspberry, HcRed-Tandem, mPlum and AQ143. The sequences and methods for their detection of the aforementioned fluorescent labels are well known to the person of skill in the art.

### Signal Detection Devices

In various embodiments of the present disclosure, a detection device is employed to measure the presence and quantity of the detected label present as part of the protein aggregate capture complex formed on the surface of the microparticles, e.g., beads. A detectable label, whether an enzyme, or a fluorescent protein, a radiolabeled isotope etc., can be detected using a visual or non-visual means. For example, a fluorescent detection label can be measured in a flow cytometer. In other devices, the assayed products can employ a fluorescent detection label that can be measured when used in conjunction with a signal detection device as non-limiting examples, spectrophotometers, fluorometers, spectrofluorimeters, fluorescence spectrophotometers, flow-cytometry detectors, or confocal microscopes when coupled with the appropriate emission and excitation sources and detectors. In some embodiments, for example, other detectable labels such as an enzyme, for example, colored substrates, chemilluminescence substrates, or radioisotope can be detected using a scintillation counter, a spectrophotometer, optical detectors and the like. For example, when it is desirable to detect the protein aggregate capture complex in a flow cytometer, a fluorescent label suitable/compatible with the particular flow cytometer may be employed.

### Assay Substrates

In various embodiments, the assay of individual samples with the components of the aggregated protein detection system described herein can be conveniently assayed in any suitable container that permits the assays described. For example, since the components are primarily in a liquid solution, the assay can be performed in a receptacle, for example, tubes, vials, microtiter plates containing a plurality of wells, wherein the reagents of the assay are mixed and contained and the detectable label can be measured either directly in the receptacle, e.g. in microtiter plates, or samples may be withdrawn from the receptacle for determination of the presence of the protein aggregate capture complex labeled with a detectable label. In various embodiments, the assay receptacle can be a microtiter plate, purely for the sake of convenience that may contain 6, 12, 24, 48, 96, 384, 1536, or 3456 sample wells arranged in a rectangular matrix, for individual testing of controls and test samples. The receptacle can be made of any material, but preferably be constructed from a suitable plastic or polymer material, for example, polystyrene, used for most optical detection microplates. It can be coloured white by the addition of titanium dioxide for optical absorbance or luminescence detection or black by the addition of carbon for fluorescent biological assays. Polypropylene and polycarbonate are commonly used materials for microplate construction as well. In preferred embodiments, the selection of receptacle material is advantageously guided by its natively low fluorescence properties.

### Arrays

Also provided by the present disclosure are multiplexed methods useful for interrogating a plurality of test samples of interest for the presence (and optionally, the number and/or proportion) of aggregated proteins, or a collection of aggregated proteins present in any one or more test samples. One example of a multiplexed method or array according to an embodiment of the present disclosure involves a heterogeneous population of microparticles, where an intrinsic fluorescent property of each subpopulation of microparticle corresponds to a specific aggregated protein (e.g., a tau protein, alpha-synuclein, hungtingtin.) disposed on the surface of the microparticles thereof. The intrinsic fluorescent property may be based, e.g., on the proportion of a first fluorochrome and a second fluorochrome on the microparticle. In this way, a single test sample from one subject may be interrogated using a panel of first capture moieties (e.g., a panel of different antibodies or antigen-binding fragments thereof, each of which specifically bind to specified epitopes and/or antigens of different aggregated proteins) to determine whether the sample provides one single type of aggregated protein or multiple different types of aggregated proteins, wherein each different capture moiety worked with one specific signalling moiety and detectable label. In effect a single microparticle with different targeting first capture moieties could be labeled with multiple detection labels to illustrate the presence of multiple aggregated proteins on a single microparticle. In some embodiments, one disease could be differentially diagnosed compared to another, or different confirmatory aggregated proteins indicative of one or more diseases can be quantitatively and/or qualitatively studied.

In other embodiments, arrays could be designed using two or more populations of microparticles, each population of microparticles having the same type of first capture moieties, with the test conditions operating with different populations of targeted microparticles, and each test sample could be interrogated to identify different protein aggregates in the same test sample.

As one of ordinary skill in the art can design based on the present disclosure, arrays can be designed as described above to interrogate a subject test sample with one set of microparticles, wherein each microparticle in the set having first capture moieties targeting different aggregated protein epitopes and/or antigens. Alternatively, one test sample can be interrogated with different sets of microparticles, wherein each set is specific for one epitope and/or antigen of one aggregated protein. Different detectable labels could be employed to signal the presence of different aggregate protein capture complexes on the surface of each microparticle or different sets of microparticles.

In some embodiments, the methods described herein may utilize a plurality or an array of reaction receptacles (e.g., microwells) to determine the presence or concentration of one or more target aggregated proteins. An array of reaction receptacles allows a fluid sample to be partitioned into a plurality of discrete reaction volumes during one or more steps of a method as described herein. In some embodiments, the reaction receptacles may all have approximately the same volume. In other embodiments, the reaction receptacles may have differing volumes.

The reaction receptacles may have any suitable volume. The volume of each individual reaction vessel (e.g., microwell) can range, for example, from nanoliters or smaller to microliters, milliliters or larger depending upon the nature of analyte molecules, the detection technique and equipment employed, and the expected concentration of the microparticles and analyte molecules in the fluid applied to the array for analysis. The size of the reaction vessel may be selected such that at the concentration of interest, between zero and one hundred microparticles, e.g., beads, would be statistically expected to be found in each reaction vessel. In a particular embodiment, the volume of the reaction vessel is selected such that at the concentration of interest, either zero or ten or more microparticles would be statistically expected to be found in a given volume each reaction vessel.

For example, in some embodiments, the reaction receptacles (e.g., wells of a microtiter plate) may have a volume between about 10 milliliters and about 10 nanoliters, between about 1 milliliter and about 100 nanoliters, between about 500 microliters and about 250 nanoliters, between about 250 microliters and about 500 nanoliters, between about 100 microliters and about 750 nanoliters or the like. In some embodiments, the reaction receptacles (e.g., microwells) have a volume of less than about 10 milliliters, less than about 1 milliliter, less than about 750 microliters, less than about 500 microliters, less than about 100 microliters, less than about 10 microliters, less than about 1 microliter, less than about 500 nanoliters, less than about 100 nanoliters, or the like. In some embodiments, the reaction receptacles (e.g., microwells) have a volume of about 0.1 nanoliters to about 10 nanoliters, about 10 nanoliters to about 500 nanoliters, about 500 nanoliters to about 1 microliter, about 1 microliter to about 100 microliters, about 100 microliters to about 500 microliters, about 500 microliters to about 1 milliliter, and from about 1 milliliter to about 10 milliliters. In particular embodiments, the reaction receptacles (e.g., microwells) have a volume ranging from about 10-5000 microliters.

For embodiments employing an array of reaction receptacles (e.g., microwells of a microtiter plate), the number of reaction receptacles in the array will depend on the composition and end use of the array. Any suitable number of reaction receptacles (e.g., microwells) can be used. Arrays containing from about 2 to many millions of reaction receptacles can be made by utilizing a variety of techniques and materials. Increasing the number of reaction receptacles in the array can be used to increase the dynamic range of an assay or to allow multiple samples or multiple types of analyte molecules to be assayed in parallel. Generally, the array will comprise between fifty and one million reaction receptacles per sample to be analyzed. In some cases, the array will comprise greater than three hundred reaction receptacles. In some embodiments, the array will comprise between about 90 to about 1,000, between about 200 and about 10,000, between about 500 and about 1,000,000, between about 100 and about 10,000, between about 1,000 and about 10,000, between about 10,000 and about 100,000, between about 100,000 and about 1,000,000, between about 50,000 and about 100,000, between about 20,000 and about 80,000, between about 30,000 and about 70,000, between about 40,000 and about 60,000, or about 50,000, reaction receptacles.

The array of reaction receptacles may be arranged on a substantially planar surface or, alternatively, in a non-planar three-dimensional arrangement. The reaction receptacles may be arrayed in a regular pattern, for example, a planar rectangular matrix, or may be randomly distributed. A preferred embodiment utilizes a regular pattern of sites on a planar structure such that the sites may be addressed in the X-Y coordinate plane. The reaction receptacles can be formed in a solid material. As will be appreciated by those in the art, the number of possible materials are very large, and include, but are not limited to, glass and modified or functionalized glass, plastics (including acrylics, polystyrene and copolymers of styrene and other materials, polypropylene, polyethylene, polybutylene, polyurethanes, TEFLON™, and the like), polysaccharides, nylon or nitrocellulose, composite materials, ceramics, and plastic resins, silica or silica-based materials including silicon and modified silicon, carbon, metals, inorganic glasses, plastics, optical fiber bundles, and a variety of other polymers. In general, the substrates allow optical detection and do not appreciably fluoresce.

Individual reaction receptacles may contain a binding surface. The binding surface may comprise essentially the entirety or only a portion of the interior surface of the reaction vessel or may be on the surface of another material or object that is confined within the reaction vessel, such as, for example, a plurality of microparticles e.g., a plurality of beads.

In one embodiment, the array of reaction receptacles is formed by mating an array of microwells with a sealing component. A microwell may be formed using a variety of techniques known in the art, including, but not limited to, photolithography, stamping techniques, molding techniques, etching techniques, or the like. As will be appreciated by those of the ordinary skill in the art, the technique used will depend on the composition and shape of the supporting material and the size and number of reaction receptacles.

In one embodiment, microfluidic systems may be used to combine microparticles with sample and subsequently with the second capture moiety and detectable label in a continuous liquid flow that bypasses the need for a reaction receptacle.

### Systems

Also provided by the present disclosure are systems. According to certain embodiments, the systems find use in practicing one or more steps of the methods of the present disclosure.

In certain aspects, the system (e.g., a flow cytometry system) is adapted to count a number of positive microparticle complexes, where the positive microparticle complexes include a microparticle, a first and second capture moiety, an aggregated protein, and a fluorescently labeled detection label. The flow cytometry system is further adapted to determine the total number of microparticle complexes acquired by the system, calculate the percentage of positive microparticle complexes among the total number of microparticle complexes, and determine the number and/or proportion of protein aggregates containing microparticles that were included in the test sample, optionally as compared to a negative (healthy) control test sample. In certain aspects, the system is further adapted to determine a mean or median fluorescence intensity of the positive microparticle complexes containing an aggregated protein acquired by the system and determine a level of the aggregated protein (that is, the size of bound aggregates) present in the test sample.

By “adapted to” is meant that the system includes the components and functionality to perform the recited determinations, calculations, etc. For example, in certain aspects, the system includes a processor and a computer-readable medium (e.g., a non-transitory computer-readable medium). The computer-readable medium includes instructions executable by the processor to, e.g., count a number of positive microparticles containing complexes, determine the total number of microparticles acquired by the system, calculate the percentage of positive microparticle aggregate protein capture complexes among the total number of microparticles, and determine the number and/or proportion of microparticles that captured the protein aggregates.

The computer-readable medium may further include instructions executable by the processor to determine a mean or median fluorescence intensity of the positive aggregate protein capture complexes acquired by the system and determine a level of the protein aggregates present in the test sample.

The computer-readable medium (or processor-readable medium) is non-transitory in the sense that it does not include transitory propagating signals per se (e.g., a propagating electromagnetic wave carrying information on a transmission medium such as space or a cable). The media and instructions may be those designed and constructed for the specific purpose or purposes. Examples of non-transitory computer-readable media include, but are not limited to: magnetic storage media such as hard disks, portable flash drives, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

### Kits and Articles of Manufacture

The invention provides kits and articles of manufacture for measuring a concentration of an aggregated protein or a plurality of different aggregated proteins as exemplified herein above (e.g., amyloid peptide, tau, alpha-synuclein, huntingtin, PrP, TDP-43, SOD1, and FUS proteins (the latter three involved for example in Amyotrophic Lateral Sclerosis (ALS)), transthyretin, immunoglobulin light chain, serum amyloid A, beta2-microglobulin, lysozyme, IAPP or amylin, crystallin, rhodopsin, hemoglobin) in a fluid sample, e.g., a test sample. In some embodiments of the present disclosure an exemplary kit comprises a plurality of microparticles comprising a first capture moiety that specifically binds an aggregate protein of interest. The kit further comprises a composition which includes a second capture moiety that specifically binds to the aggregate protein of interest, wherein the second capture moiety is coupled to a signalling moiety. The signaling moiety can be either a detectable label or a first binding partner, for example, biotin, avidin or streptavidin. In various embodiments, the first and second capture moieties can include an antibody, or antigen-binding fragment thereof or a receptor, ligand, aptamer or polynucleotide that in each case bind specifically to the same epitope or antigen, or amino acid sequence present on the aggregated protein or other aggregated species.

In some embodiments, the kit has a signalling moiety that is coupled, conjugated or covalently or non-covalently attached to a detectable label. In other embodiments, the second capture moiety is conjugated to a first binding partner, and the kit optionally further comprises a detectable label that is coupled, conjugated or covalently or non-covalently attached to a second binding partner selected from biotin, avidin or streptavidin. In each instance, the second binding partner always specifically binds to the first binding partner present with the second capture moiety. For example, in some embodiments, the second capture molecule is directly coupled to a fluorescent molecule, or coupled to biotin. In various embodiments, the detectable label can comprise a fluorescent label, a radiolabel, a luminescent agent, or a metal element label. In related embodiments, when the second detection moiety comprises a signaling moiety comprising a first binding partner, the detectable label can comprise a fluorescent label, a radiolabel, a luminescent agent, or a metal element label each of which coupled, conjugated or covalently or non-covalently attached to a second binding partner, for example, biotin, avidin or streptavidin.

In various embodiments, the kits may contain one or more reaction receptacles for permitting the admixture of the test sample with the kit components. In some embodiments, the reaction receptacle can be tubes, vessels, tissue culture plates and the like. For example, the kit may contain a blank microtiter plate having 6, 12, 24, 48, 96, 384, 1536, or 3456 sample wells arranged in a rectangular matrix. In various embodiments, the kit of the present disclosure may further comprise instructions for capturing aggregate proteins with the microparticles from a test sample using the kit components as described herein. In various embodiments, the kit may contain instructions for detecting the aggregate protein by flow cytometry.

The article or kit may include, for example, a first component containing a plurality of first capture moiety labelled microparticles (e.g., beads) and/or an array substrate comprising a plurality of reaction receptacles. The reaction receptacles may be configured to receive and contain the first capture moiety labelled microparticles. The plurality of first capture moiety labelled microparticles (e.g., beads) may have an average diameter between about 0.01 micrometer and about 100 micrometers and the size of the reaction receptacles may be selected such that only either zero or one to a million beads is able to be contained in single reaction receptacle. In some cases, the average depth of the reaction receptacles is between about 1.0 times and about 1×10⁶ times the average diameter of the beads and the average diameter of the reactions receptacles is between about 1.0 times and about 1×10⁶ times the average diameter of the beads. The average volume of the plurality of reaction receptacles may be between about 10 attoliters and about 100 picoliters, between about 1 femtoliter and about 1 picoliter, or any desired range. The substrate may comprise any number of reaction receptacles, for example, between about 12 and about 1,000,000 reaction receptacles, between about 96 and about 100,000 reaction receptacles, or between about 100,000 and about 300,000 reaction receptacles, or any other desired range. In certain embodiments, the capture probes (e.g., beads) may have an average diameter between about 1 micrometer and about 100 micrometers, or between about 1 micrometer and about 50 micrometers, or any range of sizes described therebetween.

The kits and articles of manufacture described herein may be configured to also include a second component comprising a second capture moiety comprising a signaling moiety that may or may not be directly conjugated with a detectable label and optionally a third component comprising a detectable label. The first, second and optionally third components may be provided in separate containers and instructions for their use in performing the assays of the present disclosure and/or for carrying out any of the methods or assays as described herein, e.g., in the Examples.

In some embodiments, the kit may comprise a microtiter plate containing the first capture moiety labelled microparticles with control receptacles or wells containing microparticles labelled with an irrelevant antibody to serve as a negative control.

The plurality of first capture moiety labelled microparticles (e.g., beads) provided may have a variety of properties and parameters, as described herein. For example, the beads may be magnetic. The plurality of beads may comprise a binding surface linked to one or more, different first capture moieties having two or more specificities to two or more different protein aggregates.

In some embodiments, the plurality of reactions receptacles is formed in a plate or similar substantially planar material (e.g., using lithography or other known techniques). Exemplary suitable materials are described herein, for example, microtiter plates. A microplate, also known as a microtiter plate, microwell plate, or multiwell, is a flat plate with multiple “wells” used as small test tubes. The microplate has become a standard tool in analytical research and clinical diagnostic testing laboratories. A very common usage is in the enzyme-linked immunosorbent assay (ELISA). A microplate typically has 6, 12, 24, 48, 96, 384, 1536, or 3456 sample wells arranged in a rectangular matrix. Some microplates have been manufactured with 3456 or 9600 wells, and an “array tape” product has been developed that provides a continuous strip of microplates embossed on a flexible plastic tape Each well is considered a reaction vessel as used herein to perform the described assay. In some embodiments, the well of a microplate typically holds somewhere between tens of nanolitres to several millilitres of liquid. Microplates are manufactured in a variety of materials. The most common is polystyrene, used for most optical detection microplates. It can be coloured white by the addition of titanium dioxide for optical absorbance or luminescence detection or black by the addition of carbon for fluorescent biological assays. Polypropylene and polycarbonate are commonly used for the construction of plates subject to wide changes in temperature, such as storage at −80° C. Also included are microplates constructed from solid pieces of glass and quartz.

The kit may include any of the array substrates or reaction receptacles as described herein. The kit or article may comprise any number of additional components, some of which are described in detail herein. In some cases, the article or kit may further comprise a sealing component configured for sealing the plurality of reaction receptacles. As another example, the kit may also provide solutions for carrying out an assay method as described herein. Non-limiting example of solutions include solutions containing one or more types of microparticles, for example, beads, which are surface labeled with a first capture moiety of choice. A second solution provided in the kit includes a second capture moiety that is either directly conjugated to a signalling moiety, wherein the signalling moiety is a detectable label, or the signaling moiety comprises a first binding partner such as biotin, avidin, or streptavidin and the kit optionally contains a third container comprising the second binding partner coupled, conjugated, covalently or non-covalently attached to a detectable label, wherein the first binding partner and the second binding partner form a specific bond, e.g. biotin and avidin. In some examples, the article or kit may comprise at least one type of control microparticle (e.g., a bead) surface labeled with a control antibody, or antigen binding fragment thereof that is not specific for the aggregate protein or proteins being screened.

In some embodiments, the kit may include instructions for use of components described herein. That is, the kit can include a description of use of the aggregate protein capture moiety labeled microparticles (e.g., beads) and reaction receptacles, for example, for use with a system to determine the measure of the concentration of target aggregated protein(s) in a test fluid sample. As used herein, “instructions” can define a component of instruction and/or promotion, and typically involve written instructions on or associated with packaging of the invention. Instructions also can include any oral or electronic instructions provided in any manner such that a user of the kit will clearly recognize that the instructions are to be associated with the kit. Additionally, the kit may include other components depending on the specific application, as described herein.

### Assay Methods

The present disclosure provides for assays that are designed to not only qualitatively measure the presence of a mis-folded aggregated protein that is associated with certain diseases, such as Alzheimer's disease (amyloid,tau), Parkinson's disease (alpha-synuclein), Huntington's disease (huntingtin), prion propagated disease (PrP), amyotrophic lateral sclerosis (TDP-43, SOD1, FUS, and more), as well as disease in other tissues, such the heart (cardiac amyloidosis), pancreas (type II diabetes, islet cell IAPP) cataracts (crystallin), amyloid transthyretin cardiomyopathy, some forms of atherosclerosis, hemodialysis-related disorders, and short-chain amyloidosis, among many other diseases, many of which have been exemplified herein, and those proteins that may form aggregated species in pharmacologic products. Aggregated proteins can therefore include, but not limited to: native, variant, mutant or posttranslationally modified forms of α-synuclein, tau, amyloid beta (Aβ42 and Aβ40), SOD1, TDP-43, FUS, huntingtin, transthyretin, prion proteins, crystallin, immunoglobulin light chain, serum amyloid A, beta2-microglobulin, lysozyme, gelsolin, calcitonin, prolactin, IAPP or amylin, fibrinogen, rhodopsin, glucosylceramide, hemoglobin, DNA binding proteins, RNA binding proteins and other proteins with a potential for aggregation that have been implicated, or may be suspected of involvement in, other diseases, and other proteins studied in the laboratory setting and other proteins contained in pharmacologic preparations for which protein aggregation is assessed that include preparations of antibody against CD3, against CD4, against CD11, against CD19, against CD20, against CD22, against CD30, against CD38, against CD52, against CD79b, against PD-1, against PD-L1, against PD-L2, against CCR4, against IL-1, against IL-4R, against IL-5, against IL-5R, against IL-6, against IL-6 receptor, against IL-8, against IL-13, against IL-17, against IL-17 receptor, against IL-23, against IL-33, against IL-36 receptor, against HER2, against tissue factor, against CCR4, against EGFR, agasinst PDGRFa, against IFNAR1, against sclerostin, against von Willebrand factor, against C5, against IFNgamma, against FGF23, against Factor IXa, against Kalikrein, against complement C5, against BCMA, against angiopoietin-like 3, against TROP-2, against IGF-1R, against CGRP, SLAMF7, against PCSKg, against GD2, against Nectin-4, against P-selectin, against Ebola virus, against IgE, against GD2, against BLyS, against RANK-L, against B7-H3, against MASP-2, against LAG-3, against VEGF, against alpha4beta7 integrin, against C1s, against thymic stromal lymphopoietin, against folate receptor alpha, against RSV, against CTLA-4, against FcRn, against GPIIb/IIIa, against Ep/cAM, against endotoxin, against TNF, against G protein-coupled receptor 5D, against the COVID-19 spike protein and variants thereof, against Clostridium difficile enterotoxin B, plus protein preparations of IL-2, IL-7, IL-8, IL-10, IL-12, IL-15, IL-18, IL-23, and IL-36.

These protein aggregated associated diseases all have at least one thing in common, a specific aggregated protein (or as used interchangeable herein, protein aggregates) is typically associated with or at least partially responsible for the genesis or progression of the disease. While not wishing to be bound by any particular theory, it is believed that the aggregation can be caused by chance; by protein hyperphosphorylation (a condition where multiple phosphate groups are added to the protein), by prion self-catalytic conformational conversion, by local environmental insults, or by mutations that make the protein unstable. Aggregation can also be caused by an unregulated or pathological increase in the intracellular concentration of some of these proteins. Such imbalances in protein concentration can be a consequence of mutations such as duplications of the amyloidogenic gene or changes in the protein's amino acid sequence. Imbalances can also be caused by deficiencies in the proteasome, and in autophagy, cellular machinery involved in the degradation of aging and dysfunctional proteins. In addition, some evidence suggests that the severity of these diseases correlates with an increase in oxidative stress, mitochondrial dysfunction, alteration of cytoplasmic membrane permeability, and abnormal calcium concentration Therefore, the assays of the present disclosure answer the need for robust and accurate quantitative methods to distinguish between protein monomers and aggregated oligomers, multimers, and fibrils, and to evaluate the amount and size of protein aggregates. The inventors designed this assay to be specific for the protein or molecule of interest, to distinguish protein monomer from oligomer, multimer, and fibril, and to determine both aggregate amount and size, to be performed with standard laboratory equipment, and to be neither time nor resource intensive. Successful development of a specific, simple and comparatively rapid assay for protein multimer detection and quantitation, as well as characterization of multimer size is described herein.

In some embodiments, the aggregated protein to be screened, using the methods, compositions, kits and systems of the present disclosure include, but are not limited to aggregated protein, is selected from the group consisting of native, variant, mutant or posttranslationally modified forms of α-synuclein, tau, amyloid beta (Aβ42 and Aβ40), SOD1, TDP-43, FUS, huntingtin, transthyretin, prion proteins, PrP, crystallin, immunoglobulin light chain, serum amyloid A, beta2-microglobulin, lysozyme, gelsolin, calcitonin, prolactin, IAPP or amylin, fibrinogen, rhodopsin, glucosylceramide, hemoglobin, DNA binding proteins, RNA binding proteins and other proteins with a potential for aggregation that have been implicated, or may be suspected of involvement in, other diseases, and other proteins studied in the laboratory setting and other proteins contained in pharmacologic preparations for which protein aggregation is assessed that include preparations of antibody against CD3, against CD4, against CD11, against CD19, against CD20, against CD22, against CD30, against CD38, against CD52, against CD79b, against PD-1, against PD-L1, against PD-L2, against CCR4, against IL-1, against IL-4R, against IL-5, against IL-5R, against IL-6, against IL-6 receptor, against IL-8, against IL-13, against IL-17, against IL-17 receptor, against IL-23, against IL-33, against IL-36 receptor, against HER2, against tissue factor, against CCR4, against EGFR, agasinst PDGRFa, against IFNAR1, against sclerostin, against von Willebrand factor, against C5, against IFNgamma, against FGF23, against Factor IXa, against Kalikrein, against complement C5, against BCMA, against angiopoietin-like 3, against TROP-2, against IGF-1R, against CGRP, SLAMF7, against PCSKg, against GD2, against Nectin-4, against P-selectin, against Ebola virus, against IgE, against GD2, against BLyS, against RANK-L, against B7-H3, against MASP-2, against LAG-3, against VEGF, against alpha4beta7 integrin, against C1s, against thymic stromal lymphopoietin, against folate receptor alpha, against RSV, against CTLA-4, against FcRn, against GPIIb/IIIa, against Ep/cAM, against endotoxin, against TNF, against G protein-coupled receptor 5D, against the COVID-19 spike protein and variants thereof, against Clostridium difficile enterotoxin B, plus protein preparations of IL-2, IL-7, IL-8, IL-10, IL-12, IL-15, IL-18, IL-23, IL-36 SOD1, TDP-43, rhodopsin, transthyretin, rhodopsin, IAPP or amylin, crystallin, glucosylceramide, DNA binding proteins, and other proteins with a potential for aggregation that have been implicated, or may be suspected of involvement in, other diseases, and other proteins studied in the laboratory setting and other proteins contained in pharmacologic preparations for which protein aggregation is assessed. In some embodiments, the aggregated protein is α-synuclein, tau protein, or amyloid-beta 1-42 peptide (Aβ42).

Assay methods of the present disclosure can be performed as follows:
a. providing a capture substrate comprising a microparticle conjugated to a first capture moiety;
b. blocking the capture substrate non-specific binding sites;
c. incubating the capture substrate with a test sample suspected of having the aggregated protein for a period of time sufficient for the aggregated protein to specifically bind to the first capture moiety; wherein the first capture moiety binds specifically to the aggregate protein if present, thereby forming a capture complex on the capture substrate,
d. incubating the capture substrate with a second capture moiety that specifically binds to the aggregated protein, wherein the second capture moiety is coupled to a signalling moiety, and wherein the signaling moiety comprises a detectable label; and
e. determining the amount of detectable label present on the surface of the capture substrate.

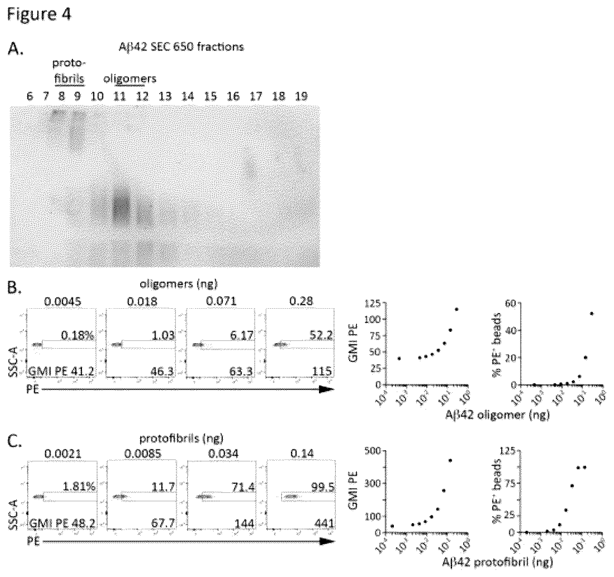

In these methods, correlating the amount of detectable label, with the presence or absence of aggregated proteins further involves analyzing a sufficient series of normal samples (1, or 2 or more samples), which may be normal clinical samples, or normal laboratory samples, or normal pharmaceutical product samples, to compare against the presence of detectable label when these normal samples are used in a control. The comparison between the test sample(s) and the control sample(s) will provide the means to define the range and limit of normal values so as to set a threshold above which test sample results may be judged to be abnormal (see FIG. 4).

Assay methods of the present disclosure for determining the presence, and the size and the amount of an aggregated protein or other aggregating species, in a test sample, to evaluate whether it is normal or abnormal, can be performed as follows:
a. providing a capture substrate comprising a microparticle conjugated to a first capture moiety;
b. blocking the capture substrate non-specific binding sites;
c. incubating the capture substrate with a test sample suspected of having the aggregated protein or other aggregating species for a period of time sufficient for the aggregated protein to specifically bind to the first capture moiety; wherein the first capture moiety binds specifically to the aggregate protein or other aggregating species if present, thereby forming a capture complex on the capture substrate,
d. incubating the capture substrate with a second capture moiety that specifically binds to the aggregated protein or other aggregating species, wherein the second capture moiety is coupled to a signaling moiety, and wherein the signaling moiety comprises a detectable label; and
e. determining the amount of detectable label present on the surface of the capture substrate; and
f. analyzing a sufficient series of normal samples, which may be normal clinical samples, or normal laboratory samples, or normal pharmaceutical product samples, will provide the means to define the range and limit of normal values so as to set a threshold above which test sample results may be judged to be abnormal (see FIG. 4).

Assay methods of the present disclosure for determining the presence, and the size and the amount of an aggregated protein or other aggregating species, in a test sample, to evaluate whether it is normal or abnormal, by quantitatively reporting the size of the aggregate, the method comprising:
a. providing a capture substrate comprising a microparticle conjugated to a first capture moiety;
b. blocking the capture substrate non-specific binding sites;
c. incubating the capture substrate with a test sample suspected of having the aggregated protein or other aggregating species for a period of time sufficient for the aggregated protein to specifically bind to the first capture moiety; wherein the first capture moiety binds specifically to the aggregate protein or other aggregating species if present, thereby forming a capture complex on the capture substrate,
d. incubating the capture substrate with a second capture moiety that specifically binds to the aggregated protein or other aggregating species, wherein the second capture moiety is coupled to a signaling moiety, and wherein the signaling moiety comprises a detectable label; and
e. comparing the amount of detectable label present on the surface of the capture substrate with a standard curve in such a way that the intensity of the signaling moiety can be correlated with molecular size.

Methods for correlating the molecular size of the aggregated protein under investigation to the amount and/or intensity of the detectable label can include, as follows:
f. a series of molecules of known molecular sizes ranging from 10,000 Da to 1,000,000 Da or 20,000 Da to 4,000,000 Da will be subjected to sedimentation equilibrium by ultracentrifugation and at the same time a heterogeneous mixture of aggregated protein or other aggregating species will be subjected to sedimentation equilibrium by ultracentrifugation. Fractions will be collected from both. Fractions from the molecules of known molecular sizes will be analyzed for the presence of the molecules in each fraction by ELISA or Western blot, which will define the level of molecular mass sedimenting in each fraction. Fractions from the heterogeneous mixture of aggregated protein or other aggregating species will be analyzed by immunocapture microparticle (“bead”) assay and the level of the signal produced by each fraction recorded;
g. the two values are then correlated to provide an indication of the molecular mass attendant to a given signal value. The molecular mass provides an indication of the number of repetitive units by simple division of the known molecular mass of monomeric aggregating species; and
h. analysis of multiple normal samples, which may be normal clinical samples, or normal laboratory samples, or normal pharmaceutical product samples, will provide the means to define the range and limit of normal size values so as to set a threshold above which test sample results may be judged to be abnormal (see FIG. 4).

In related embodiments, the present disclosure provides for a method for determining the presence, and the size and the amount of an aggregated protein or other aggregating species, in a test sample, to evaluate whether it is normal or abnormal, by reporting the relative size of the aggregate, the method comprising:
a. providing a capture substrate comprising a microparticle conjugated to a first capture moiety;
b. blocking the capture substrate non-specific binding sites;
c. incubating the capture substrate with a test sample suspected of having the aggregated protein or other aggregating species for a period of time sufficient for the aggregated protein to specifically bind to the first capture moiety; wherein the first capture moiety binds specifically to the aggregate protein or other aggregating species if present, thereby forming a capture complex on the capture substrate,
d. incubating the capture substrate with a second capture moiety that specifically binds to the aggregated protein or other aggregating species, wherein the second capture moiety is coupled to a signaling moiety, and wherein the signaling moiety comprises a detectable label; and
e. comparing the amount of detectable label present on the surface of the capture substrate with a standard curve in such a way that the intensity of the signaling moiety can be correlated with relative molecular size.

Methods for correlating the molecular size of the aggregated protein under investigation to the amount and/or intensity of the detectable label can include, as follows: the protein that aggregates or other aggregating species will be aggregated in vitro to different levels of size by altering the time and conditions of aggregation and these samples will then be subjected to size exclusion chromatography and resultant fractions will be analyzed by immunocapture microparticle (“bead”) assay and the level of the signal produced by each fraction recorded; the signal produced by test samples subjected to immunocapture microparticle (“bead”) assay can then be related to a particular fraction; analysis of multiple normal samples will provide the means to define the range and limit of normal fraction values so as to set a threshold above which test sample results may be judged to be abnormal (see FIG. 4).

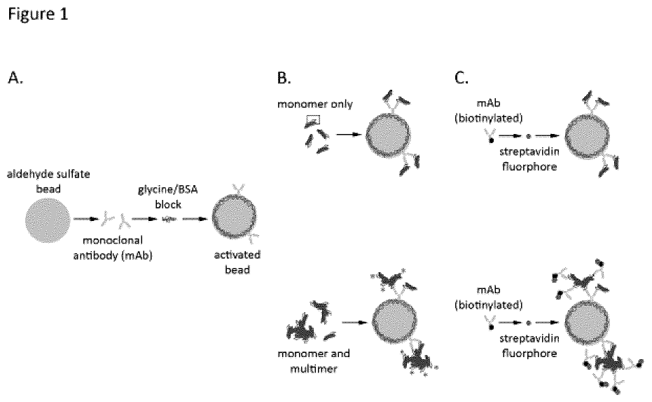

In one illustrative embodiment, an exemplary microparticle immunocapture assay for protein aggregation described herein involves three steps (See FIG. 1). A functionalized microparticle, for example, aldehyde sulfate functionalized beads are utilized upon which an epitope-specific, monoclonal capture antibody is bound by covalent interaction (FIG. 1A) to form the first capture moiety. After blocking with a non-specific blocking agent or blocker, (for example, bovine serum albumin (BSA), human serum albumin, fetal bovine serum, non-fat milk proteins, casein, fish gelatin, polyethylene glycol, polyvinyl alcohol, or polyvinylpyrrolidone, other non-specific proteins and non-specific DNA or non-specific RNA), the loaded beads are incubated with a test sample containing the protein of interest, (here an aggregated protein of interest) leading to antibody-protein interaction (FIG. 1B, binding epitope, box) and forming an aggregate protein capture complex. The subsequent protein capture and presentation occurs on the bead surface. No unoccupied binding sites should exist on any captured monomeric proteins due to antibody binding the single epitope, whereas unoccupied binding sites, or detection sites, will exist on captured protein aggregates (multimers) (FIG. 1B, asterisks). A subsequent two-step incubation, first with a second capture moiety, for example, the identical monoclonal antibody used as the first capture moiety, but which is (biotinylated), and then a second incubation with streptavidin (SA) conjugated to a fluorophore which allows detection of only the protein aggregates (multimers), while bound monomers go undetected (FIG. 1C). More unoccupied detection sites in larger multimers (larger aggregated protein) result in more antibody/Streptavidin-fluorophore binding, and hence greater fluorescence. The fluorescence signal on individual beads is then quantitated by a detection device, for example a flow cytometer acquisition and analysis.

In some embodiments, the assay can be performed in single plates or in a multiplex fashion, where a plurality of plates with one, tens, twenties, thirties, fourties, fifties, sixties, seventies, eighties, nineties, hundreds, thousands, tens of thousands, hundreds of thousands and millions of test samples can be interrogated using the assays described above. In various embodiments assay substrates, for example, microtubes or microplates, each with a plurality of test and control wells are provided. Capture substrates can be prepared using microparticles of various shapes and sizes. For each test sample, a plurality of microparticles, for example beads, are provided directly with the appropriate functionalized surface for direct or indirect coupling of the first capture moiety, wherein each microparticle of the plurality of microparticles may be labelled with one, two, three, four, or more different binding first capture moieties, wherein each type of first capture moiety binds specifically to one type of aggregated protein. In this fashion, the single test well containing the capture substrate and microparticles can bind to one aggregated protein or multiple aggregated proteins in parallel and the assay can be multiplexed to screen for a plurality of aggregated proteins in each test sample.

In various embodiments, the microparticles labelled with one or more first capture moieties are then optionally washed and then blocked with a blocking material that binds to coupling sites on the surface of the microparticles. Any blocking agent can be used, for example, skim milk or BSA. Then the prepared microparticles are then mixed with a test sample to determine the quantity and/or size of the aggregated proteins that may be present in the test sample. In various embodiments, the microparticles and test samples and matching controls if any, can be incubated at temperatures ranging from about 4° C. to about 50° C., preferably from about 15° C. to about 37° C., and any degree within this range, for a period of about one minute to about 300 minutes, preferably from about 15 minutes to about 120 minutes, and most preferably from about 30 minutes to about 60 minutes. Once incubated with the test sample, the microparticles may optionally be washed before incubating the microparticles with the second capture moiety. Since the first capture moiety and the second capture moiety bind to the same antigen, epitope or sequence of amino acids in the aggregated protein, the incubation parameters of the second capture moiety can be same as the conditions used with the first capture moiety. In some illustrative examples, the microparticles incubated with the test or control samples are then incubated with the second capture moiety at temperatures ranging from about 4° C. to about 50° C., preferably from about 15° C. to about 37° C., and any degree within this range, for a period of about one minute to about 300 minutes, preferably from about 15 minutes to about 120 minutes, and most preferably from about 30 minutes to about 60 minutes. The first and second capture moieties are added to the microparticles, for example in 1 μL of microparticles ranging in size from about 0.01 μm to about 100 μm are coated and/or added with 0.001 μg to about 100 μg of the first capture moiety and/or second capture moiety.

After the microparticles have been incubated with the second capture moiety, and/or detectable label, the microparticles are optionally washed one to five times or more to remove unbound second capture moiety and/or detectable label. In some embodiments, the second capture moiety is coupled to a detectable label and the sample can be directly processed to determine the amount of detectable label present on the surface of the microparticles using an appropriate detection device, for example, a colorimeter, a fluorometer, a scintillation counter, a flow cytometer, or a confocal microscope with an appropriate radioisotope, fluorescence or illuminescence detection device.

In other embodiments, where the second capture moiety is coupled to a first binding partner, such as biotin, avidin, or streptavidin, the microparticles are incubated with a detectable label, such as an enzyme, or a fluorescent protein, a radiolabeled isotope etc. that is coupled with a second binding partner such as biotin, avidin, or streptavidin, provided that the first binding partner and the second binding partners are bound specifically, for example, biotin and avidin or biotin and streptavidin binding pairs. The further incubation with a detectable label with a second capture moiety can proceed at temperatures ranging from about 4° C. to about 50° C., preferably from about 15° C. to about 37° C., and any degree within this range, for a period of about one minute to about 300 minutes, preferably from about 15 minutes to about 120 minutes, and most preferably from about 30 minutes to about 60 minutes.

After incubation with the second capture moiety and the detectable label, the microparticles can be examined to measure the quantity and size of aggregated protein captured on the surface of the microparticles. The emitted label from the detection label from each test sample, may then be compared to known signal emitted amounts and compared to standard curves etc. to determine the amount and size of the aggregated proteins present in the test sample.

### Protein Aggregates (or Aggregated Proteins) Found in Relevant Disease Models

In various embodiments, test samples containing materials including one or more proteins that may form aggregated proteins can be assessed using the assays and compositions, kits and systems described herein. Protein aggregates to be measured and analyzed include a wide variety of proteins that are either pathogenic, for example aggregated proteins that are associated with proteinopathy diseases described in greater detail below, or may include other proteins that aggregate and form undesirable aggregates in laboratory solutions and pharmaceutical preparations. Other aggregates of importance may include tissue entrapped protein aggregates that can be processed and analyzed in liquid form.

The present disclosure also contemplates DNA/RNA binding protein aggregates that can cause disease and conditions that require medical attention. The quantification and assessment of these DNA/RNA binding proteins, for example, proteins that bind DNA are implicated in Systemic Lupus Erythematosus when anti-dsDNA antibodies form protein aggregates and cause tissue damage in the kidneys and other tissues. RNA-binding proteins (RBPs) have also been implicated in protein aggregate proteinopathies. RBPs seldom act alone but rather form extensive protein-protein and protein-RNA interactions in an immense number of permutations that allow for spatial and temporal control of gene expression in response to a range of stimuli. Aggregations are hallmarks of many but not all neurodegenerative diseases, and aggregated TDP-43 inclusions represent the single greatest unifying factor throughout ALS molecular pathology. TDP-43 proteinopathy is also predominantly associated with frontotemporal lobar dementia (FTLD) subtype Ub+ (FTLD-U)/TDP+ (FTLD-TDP), a pathological form of the heterogeneous dementia marked by the loss of cortical (and other) neurons. There is considerable intrafamily and clinical overlap between ALS and FTD, particularly FTLD-TDP, and the two diseases are thought to be different clinical manifestations of a common pathological mechanism. The presence of aggregated TDP-43 in both familial and sporadic ALS with or without accompanying TDP-43 mutations represents a central paradox in the effort to characterize the molecular events that are necessary and/or sufficient to cause disease.

TDP-43 is a ubiquitously expressed critical protein. It is highly conserved across species, and genetic knockout leads to embryonic lethality in mice. TDP-43 negatively regulates its own mRNA expression by binding its 3′ untranslated region (UTR), an interaction that requires a C-terminal Gly-rich region. Tight regulation of TDP-43 levels is thus important to the cell; one consequence of elevated TDP-43 expression is increased skipping of exon 9 in the mRNA encoding the cystic fibrosis transmembrane regulator, among other similar roles in splicing inhibition. Overexpression of TDP-43 can be toxic in a wide array of cells, yeasts, and animals, including humans.

### Proteinopathies

The term proteinopathy refers to diseases, disorders, and/or conditions that is associated with the pathogenic accumulation and/or aggregation of one or more types of proteins. In some embodiments, a proteinopathy may involve pathological alterations in one or more of protein production, folding, metabolism, degradation (e.g., autophagic, lysosomal, proteosomal), transportation or trafficking, secretion, etc. Autophagy may include microautophagy, macroautophagy, chaperone-mediated autophagy, mitophagy, pexophagy.

In some embodiments, a proteinopathy may involve efficiency of transport or the ability of a protein to be transported out of the endoplasmic reticulum to its native location within cell, cell membrane, or into the extracellular environment. For example, the native location of a lysosomal enzyme is the lysosome. The regular trafficking pathway for a lysosomal protein comprises: endoplasmic reticulum then Golgi apparatus then endosomes then lysosomes, but, in general, mutant proteins and/or certain wild-type proteins whose folding and trafficking may be incomplete would be unstable in the endoplasmic reticulum and their trafficking along a normal transport pathway would be retarded.

In some embodiments, a proteinopathy may involve regulatory intracellular signaling pathways. For example, in some embodiments, temporal cellular proteostasis adaptation is necessary, due to the presence of an ever-changing proteome during development and the presence of new proteins and the accumulation of misfolded proteins upon aging. Because the fidelity of the proteome is challenged during development and aging, and by exposure to pathogens that demand high protein folding and trafficking capacity, cells utilize stress sensors and inducible pathways to respond to a loss of proteostatic control. These include the heat shock response (HSR) pathway that regulates cytoplasmic proteostasis, unfolded protein response (UPR) pathway that helps maintain exocytic pathway proteostasis, the calcium ion (Ca²⁺) signaling pathway, and/or pathways associated with organismal longevity including, insulin/insulin growth factor receptor signaling pathway and pathways associated with dietary restriction as well as processes associated with the mitochondrial electron transport chain process.

HSR pathway refers to enhanced expression of heat shock proteins (chaperone/cochaperone/folding enzymes) in the cytosol that can have an effect on proteostasis of proteins folded and trafficked within the secretory pathway as a soluble lumenal enzyme. Cytosolic factors including chaperones are likely essential for adapting the secretory pathway to be more folding and trafficking permissive (Bush et al., J Biol Chem 272:9086, 1997; Liao et al., J Cell Biochem 99:1085, 2006; Westerheide et al., J Biol Chem 279:56053, 2004).

UPR pathway refers to a stress sensing mechanism in the endoplasmic reticulum (ER) wherein the ER responds to the accumulation of unfolded proteins in its lumen by activating up to three integrated arms of intracellular signaling pathways, e.g., UPR-associated stress sensors, IRE1, ATF6, and PERK, collectively referred to as the unfolded protein response, that regulate the expression of numerous genes that function within the secretory pathway (Ron et al., Nat Rev Mol Cell Biol 8:519, 2007; Schroeder et al., Ann Rev Biochem 74:739, 2005). UPR associated chaperones include, but are not limited to BiP, GRP94, and calreticulin.

The Ca²⁺ ion is a universal and important signaling ion in the cell. Ca²⁺ signaling affects numerous cellular functions by diverse pathways and is a primary regulator of endoplasmic reticulum (ER) function (Berridge et al., Nat Rev Mol Cell Biol 4:517, 2003; Burdakov et al., Cell Calcium 38:303, 2005; Gorlach et al., Antioxid Redox Signal 8:1391, 2006). Ca²⁺ homeostasis is also modulated by the activity of ER calcium receptors. ER calcium receptors include, for example, ryanodine receptors (RyR), inositol 3-phosphate receptors (IP3R) and sarcoplasmic/endoplasmic calcium (SERCA) pump proteins. RyR and IP3R mediate efflux of calcium from the ER whereas SERCA pump proteins mediate influx of calcium into the ER. There are three RyR subtypes, RyR1, RyR2 and RyR3. Emerging evidence indicates that calcium signaling may influence proteinopathic diseases, disorders, and/or conditions. This hypothesis is supported by observations that manipulation of calcium homeostasis by SERCA pump inhibitors, such as thapsigargin enhances folding and trafficking of the δF508 cystic fibrosis transmembrane conductance regulator (CFTR) and curcumin.

In some embodiments, the present invention provides a method directed to identifying and quantifying the level of aggregated α-synuclein levels in a sample from a subject suspected of having a neurodegenerative disease. In some embodiments, the subject is first diagnosed as having an increased level of α-synuclein prior to performing the assay with the subject's test sample, or the subject is at increased risk of having increased α-synuclein levels.

In some embodiments, proteinopathy may involve lipid accumulation. For example, pathological accumulations of lactosylceramide, glucosylceramide (GlcCer), G_{M2}-ganglioside, and asialo-G_{M2} are found in Nieman-Pick Type C disease, which is a lysosomal cholesterol storage disease that is not associated with deficient acid sphingomyelinase due to missense mutations in the gene encoding the enzyme (Vanier et al., Brian Pathology 8:163-74, 1998). Without wishing to be bound by any particular theory, Applicants note that a variety of mechanisms have been proposed to explain this accumulation including, for example, defective lipid trafficking. A healthy endosomal trafficking system is critical to neuronal function. Disruption of glycosphingolipid metabolism, including GlcCer, impairs cellular trafficking and causes cholesterol sequestration and accumulation. Accumulated glycolipids form “lipid rafts” that can sequester proteins important in maintaining normal trafficking in the endosomal system. Moreover, the defective trafficking of lipids observed in fibroblasts from Niemann-Pick Type C cells can be reversed by treatment with a potent inhibitor of glycosphingolipid biosynthesis (Lachmann et al., Neurobiol Dis. 16 (3): 654, 2004), further underscoring the involvement of GlcCer and other lipids in the pathology of this disease. For example, inhibition of glucosylceramide synthase, the enzyme that catalyzes the first step in the biosynthesis of glycosphingolipids delays onset of a proteinopathic disease, disorder, and/or condition through the following potential mechanisms: substrate reduction; diminished aggregation of a protein (e.g., α-synuclein); act as an anti-inflammatory agent; or inhibit non-lysosomal GCase resulting in altered levels of neuronal glycosphingolipids.

Further, association with lipid rafts is required for normal localization of α-synuclein to its native cellular location, the synapses. Mutations associated with the pathology of Parkinson's disease disrupt this association. Thus, changes in lipid raft composition that also disrupt this association could contribute to Parkinson's disease by impairing normal localization and distribution of α-synuclein.

Exemplary proteins whose aggregation is observed in certain proteinopathies include α-synuclein (synucleinopathies such as Parkinson's diseases (PD) and Lewy body disease), tau proteins (tauopathies such as Alzheimer's Disease), amyloid beta proteins (amyloidopathies such as vascular dementia, cognitive impairment, and Alzheimer's Disease), SOD1 (SOD1 proteinopathies such as amyotrophic lateral sclerosis), TDP-43 (TDP-43 proteinopathies such as amyotrophic lateral sclerosis), huntingtin (Huntington's disease), rhodopsin (retinitis pigmentosa), IAPP or amylin (diabetes), crystallin (cataracts), transthyretin (systemic amyloidosis), and/or a number of proteins (e.g., glucosylceramide) in the case of the diseases collectively known as lysosomal storage disease. It will be appreciated by those of ordinary skill in the art that certain diseases, disorders, and/or conditions are associated with misfolding and/or aggregation of more than one different protein.

In some embodiments, the present invention provides methods for qualitatively identifying and quantifying protein aggregates that are observed in a variety of different types of disorders, diseases, and/or conditions, including cognitive impairment disorders, proliferative diseases, inflammatory diseases, cardiovascular diseases, immunologic diseases, ocular diseases, mitochondrial diseases, neurodegenerative diseases, hematologic and oncologic diseases, and lysosomal storage diseases. Some embodiments of the present invention are applicable to all proteinopathies, particularly where there are specific reagents, such as first and second capture moieties that are specific for the aggregated protein in each of these diseases and proteinopathies. Assays for Screening Protein Aggregates Associated With Neurodegenerative and Non-degenerative Diseases. The present disclosure provides for assays that are designed to not only qualitatively and quantitatively measure the presence of a mis-folded aggregated protein that is associated with certain neurodegenerative diseases, such as Alzheimer's disease (amyloid, tau), Parkinson's disease (alpha-synuclein), Huntington's disease (huntingtin), prion propagated disease (PrP), amyotrophic lateral sclerosis (TDP-43, SOD1, FUS, and more), as well as non-neurodegenerative diseases in other tissues, such the heart (cardiac amyloidosis), and pancreas (type II diabetes, islet cell IAPP) cataracts, amyloid transthyretin cardiomyopathy, some forms of atherosclerosis, hemodialysis-related disorders, and short-chain amyloidosis, among many others. These diseases all have at least one thing in common, a specific aggregated protein (or as used interchangeable herein, protein aggregates) is typically associated with or at least partially responsible for the genesis or progression of the disease.

In some embodiments, the diseases that can be identified using the assays of the present disclosure, include, but are not limited to: Alzheimer's Disease (AD), Parkinson's Disease (PD), Huntington's Disease (HD), Prion Disease, Creutzfeldt-Jakob disease, amyotrophic lateral sclerosis (ALS), cardiac amyloidosis and diabetes mellitus type I.

The present invention provides methods, compositions, kits and arrays for the qualitative and quantitative assessment of dysregulated aggregate protein accumulation associated with neurodegenerative and non-degenerative diseases.

Many neurodegenerative diseases are linked to intracellular and/or extracellular accumulation of specific protein aggregates. In many cases, it is thought that the protein aggregates exert toxic effects on the brain and contribute to disease pathology.

Neurodegenerative proteinopathies are typically associated with aggregates in the following structures: cytosol, e.g., PD and Huntington's disease; nucleus, e.g., spinocerebellar ataxia type 1 (SCA1); endoplasmic reticulum (ER), e.g., familial encephalopathy with neuroserpin inclusion bodies; extracellular proteins, e.g., amyloid beta in Alzheimer's disease (AD). Mitochondrial dysfunction and oxidative stress can also play a role in neurodegenerative disease pathogenesis (Lin et al., Nature 443:787, 2006).

### Synucleinopathies

The present invention provides methods, compositions, kits and arrays for the qualitative and quantitative assessment of dysregulated aggregate accumulation associated with synucleinopathies. Synucleinopathies are a diverse group of neurodegenerative proteinopathies that share common pathological lesions composed of aggregates of conformational and posttranslational modification of the protein α-synuclein in certain populations of neurons and glia.

PD is a neurodegenerative movement disorder characterized by the accumulation of the pre-synaptic α-synuclein protein in the form of Lewy body inclusions. Other neurodegenerative disorders characterized by α-synuclein accumulation include, multiple systems atrophy, dementia with Lewy bodies, and Lewy body mutant of Alzheimer's disease. Pathological α-synuclein is also recognized as a subset of the proteinacious lesions detected in neurodegeneration with brain iron accumulate type I, amyotrophic lateral sclerosis/Parkinson's dementia complex of Guam, and familial AD.

Certain evidence proposes that α-synuclein interacts and accelerates the aggregation of tau, another aggregation-prone protein of the central nervous system that is found in neurofibrillary tangles that characterize sporadic AD (Giasson et al., Sci. Aging Knowl. Environ. 18: orb, 2003). Several mutations in α-synuclein, all which stabilize and accelerate protein aggregation, have been found in rare familial forms of PD (Hardy et al., Am. J. Epidmeiol. 164 (2): 126, 2006). Several in vivo and cell culture models have demonstrated that overexpression and aggregation of α-synuclein cause neurotoxicity (Dawson et al., Neuron 66:646, 2010).

Synucleins are small proteins (123 to 143 amino acids), and the primary structure is usually divided into three distinct domains: an amphipahtic N-terminal region characterized by negative imperfect repeats of the consensus sequence KTKEGV. This sequence results in all synucleins having in common a highly conserved α-helical lipid-binding motif; a central hydrophobic region which includes the non-Aβ component of Alzheimer's disease amyloid plaque (NAC) region involved in protein aggregation; and a highly acidic and proline-rich C-terminal region that has no distinct structural propensity.

Human synuclein family members include α-synuclein, β-synuclein, and γ-synuclein and all synuclein genes are relatively well conserved both within and between species (Cookson M R, Molecular Neurodegeneration 4 (9): 1750, 2009). The most recently cloned synuclein protein, synoretin has a close homology to γ-synuclein, and is predominantly expressed in the retina.

α-synuclein, also referred to as non-amyloid component of senile plaques precursor protein (NACP), SYN 1 or synelfin, is a heat-stable, “natively unfolded” protein of poorly defined function. It is predominantly expressed in the central nervous system (CNS) neurons where it is localized to presynaptic terminals. Electron microscopy analysis have suggested that α-synuclein is localized in close proximity to synaptic vesicles at axonal termini, pointing to a role for α-synuclein in neurotransmission or synaptic organization. Further, biochemical analysis has revealed that a small fraction of «-synuclein may be associated with vesicular membranes, but most α-synuclein is cytosolic.

Genetic and histopathological evidence supports the idea that α-synuclein is the major component of several proteinaceous inclusions characteristic of specific neurodegenerative diseases. Pathological synuclein aggregations are restricted to the α-synuclein isoforms, as β and γ-synucleins have not been detected in these inclusions. The presence of α-synuclein positive aggregates is disease specific. Lewy bodies, neuronal fibrous cytoplasmic inclusions that are histopathological hallmarks of PD and DLBD are strongly labeled with antibodies to α-synuclein. Dystrophic ubiquitin-positive neurites associated with PD pathology, termed Lewy neurites (LN) and CA2/CA3 ubiquitin neurites are also α-synuclein positive. Furthermore, pale bodies, putative precursors of LBs, thread-like structures in the perikarya of slightly swollen neurons and glial silver positive inclusions in the midbrains of patients with LB diseases are also immunoreactive for α-synuclein. α-synuclein is likely the major component of glial cell inclusions (GCIs) and neuronal cytoplasmic inclusions in MSA and brain iron accumulation type 1 (PANK1). α-synuclein immunoreactivity is present in some dystrophic neurites in senile plaques in Alzheimer's Disease (AD) and in the cord and cortex in ALS. α-synuclein immunoreactivity is prominent in transgenic and toxin-induced mouse models of Parkinson's Disease (PD), Alzheimer's Disease (AD), Amyotrophic Lateral Sclerosis (ALS), and Huntington's Disease (HD).

Further evidence supports the notion that α-synuclein is the actual building block of the fibrillary components of LBs, LNs, and GCIs. Immunoelectron microscopic studies have demonstrated that these fibrils are intensely labeled with α-synuclein antibodies in situ. Sarcosyl-insoluble α-synuclein filaments with straight and twisted morphologies can also be observed in extracts of DLBD and MSA brains. Moreover, α-synuclein can assemble in vitro into elongated homopolymers with similar widths as sarcosyl-insoluble fibrils or filaments visualized in situ. Polymerization is associated with a concomitant change in secondary structure from random coil to anti-parallel-sheet structure consistent with the Thioflavine-S reactivity of these filaments. Furthermore, the PD-association with α-synuclein mutation, A53T, may accelerate this process, as recombinant A53T α-synuclein has a greater propensity to polymerize than wild-type α-synuclein. This mutation also affects the ultrastructure of the polymers; the filaments are slightly wider and are more twisted in appearance, as if assembled from two protofilaments. The A30P mutation may also modestly increase the propensity of α-synuclein to polymerize, but the pathological effects of this mutation also may be related to its reduced binding to vesicles. Interestingly, carboxyl-terminally truncated α-synuclein may be more prone to form filaments than the full-length protein.

Current treatment options for synucleinopathic diseases include symptomatic medications such as carbidopa-levodopa, anticholinergics, and monoamine oxidase inhibitors, with widely variable benefit. Even for the best responders, i.e., patients with idiopathic Parkinson's disease, an initial good response to levodopa is typically overshadowed by drug-induced complications such as motor fluctuations and debilitating dyskinesia, following the first five to seven years of therapy. For the rest of the disorders, the current medications offer marginal symptomatic benefit. Given the severe debilitating nature of these disorders and their prevalence, there is a clear need in the art for novel approaches towards treating and managing synucleinopathies. In certain embodiments, the synucleinopathy is Parkinson's disease, diffuse Lewy body disease, and/or multiple system atrophy disorder.

### Parkinson's Disease (PD)

In some embodiments, the present invention specifically provides methods, compositions, kits and arrays for the qualitative and quantitative assessment of dysregulated aggregate accumulation associated with PD, a synucleinopathy. PD is a neurodegenerative disorder characterized by bradykinesia, rigidity, tremor, and postural instability. The pathologic hallmark of PD is loss of neurons in the substantia nigra pars compacta (SNpc) and the appearance of Lewy bodies in remaining neurons. It appears that more than about 50% of the cells in the SNpc need to be lost before motor symptoms appear. Associated symptoms often include small handwriting (micrographia), seborrhea, orthostatic hypotension, urinary difficulties, constipation and other gastrointestinal dysfunction, sleep disorders, depression and other neuropsychiatric phenomena, dementia, and smelling disturbances (occurs early). Patients with Parkinsonism have greater mortality, about two times compared to general population without PD. This is attributed to greater frailty or reduced mobility.

Diagnosis of PD is mainly clinical and is based on the clinical findings listed above. Parkinsonism refers to any combination of two of bradykinesia, rigidity, and/or tremor. PD is the most common cause of parkinsonism. Other causes of parkinsonism are side effects of drugs, mainly the major tranquilizers, such as Haldol, strokes involving the basal ganglia, and other neurodegenerative disorders, such as DLBD, progressive supranuclear palsy (PSP), frontotemporal dementia (FTD), MSA, and Huntington's disease. The pathological hallmark of PD is the Lewy body, an intracytoplasmatic inclusion body typically seen in affected neurons of the substantia nigra and to a variable extent, in the cortex. Recently, α-synuclein has been identified as the main component of Lewy bodies in sporadic Parkinsonism.

Although parkinsonism can be clearly traced to viruses, stroke, or toxins in a few individuals, in many cases, the etiology of Parkinson's disease is unknown. Environmental influences which may contribute to PD may include drinking well water, farming and industrial exposure to heavy metals (e.g., iron, zinc, copper, mercury, magnesium and manganese), alkylated phosphates, and orthonal chlorines. Paraquat (an herbicide) has also been associated with increased prevalence of Parkinsonism including PD. Cigarette smoking is associated with a decreased incidence of PD. The current consensus is that PD may either be caused by an uncommon toxin combined with high genetic susceptibility or a common toxin combined with relatively low genetic susceptibility.

Some subjects that are at risk of developing PD can be identified for example by genetic analysis. There is good evidence for certain genetic factors being associated with PD. Large pedigrees of autosomal dominantly inherited PDs have been reported. For example, a mutation in α-synuclein is responsible for one pedigree and triplication of the SNCA gene (the gene coding for α-synuclein) is associated with PD in others.

### Diffuse Lewy Body Disease and Rapid Eye Movement Sleep Disorder

In some embodiments, the present invention specifically provides methods, compositions, kits and arrays for the qualitative and quantitative assessment of dysregulated aggregate accumulation associated with DLBD, a synucleinopathy. DLBD is the second most common cause of neurodegenerative dementia on older people, it affects 7% of the general population older than 65 years and 30% of those aged over 80 years. It is part of a range of clinical presentations that share a neurotic pathology based on normal aggregation of the synaptic protein α-synuclein. DLBD has many of the clinical and pathological characteristics of the dementia that occurs during the course of PD. In addition to other clinical and neurologic diagnostic criteria, a “one year rule” can been used to separate DLBD from PD. According to this rule, onset of dementia within 12 months of Parkinsonism qualifies as DLBD, whereas more than 12 months of Parkinsonism before onset of dementia qualifies as PD. The central features of DLBD include progressive cognitive decline of sufficient magnitude to interfere with normal social and occupational function. Prominent or persistent memory impairment does not necessarily occur in the early stages, but it is evident with progression in most cases. Deficits on tests of attention and of frontal cortical skills and visual spatial ability can be especially prominent core diagnostic features, two of which are essential for diagnosis of probable and one for possible DLBD are fluctuating cognition with pronounced variations in attention and alertness, recurrent visual hallucinations that are typically well-formed and detailed, and spontaneous features of Parkinsonism. In addition, there can be some supportive features, such as repeated falls, syncope, transient loss of consciousness, neuroleptic sensitivity, systematized delusions, hallucinations and other modalities, REM sleep behavior disorder, and depression. Patients with DLBD do better than those with Alzheimer's Disease in tests of verbal memory, but worse on visual performance tests. This profile can be maintained across the range of severity of the disease but can be harder to recognize in the later stages owing to global difficulties. DLBD typically presents with recurring episodes of confusion on a background of progressive deterioration. Patients with DLBD show a combination of cortical and subcortical neuropsychological impairments with substantial attention deficits and prominent frontal subcortical and visual spatial dysfunction. These help differentiate this disorder from Alzheimer's disease.

Rapid eye movement (REM), sleep behavior disorder is a parasomnia manifested by vivid and frightening dreams associated with simple or complex motor behavior during REM sleep. This disorder is frequently associated with the synucleinopathies, DLBD, PD, and MSA, but it rarely occurs in amyloidopathies and tauopathies. The neuropsychological pattern of impairment in REM sleep behavior disorder/dementia is similar to that reported in DLBD and qualitatively different from that reported in Alzheimer's disease. Neuropathological studies of REM sleep behavior disorder associated with neurodegenerative disorder have shown Lewy body disease or multiple system atrophy. REM sleep wakefulness disassociations (REM sleep behavior disorder, daytime hypersomnolence, hallucinations, cataplexy) characteristic of narcolepsy can explain several features of DLBD, as well as PD. Sleep disorders could contribute to the fluctuations typical of DLBD, and their treatment can improve fluctuations and quality of life. Subjects at risk of developing DLBD can be identified. Repeated falls, syncope, transient loss of consciousness, and depression are common in older people with cognitive impairment and can serve (as a red flag) to a possible diagnosis of DLBD. By contrast, narcoleptic sensitivity in REM sleep behavior disorder can be highly predictive of DLBD. Their detection depends on the clinicians having a high index of suspicion and asking appropriate screening questions.

Clinical diagnosis of synucleinopathic subjects that are affected by or at risk of developing LBD can be supported by the methods and assays of the present disclosure followed by confirmatory neuroimaging investigations. Changes associated with DLBD include preservation of hippocampal, and medial temporal lobe volume on magnetic resonance imaging (MRI) and occipital hypoperfusion on single-photon emission computed tomography (SPECT). Other features, such as generalized atrophy, white matter changes, and rates of progression of whole brain atrophy are not helpful in differential diagnosis. Dopamine transporter loss in the caudate and putamen, a marker of nigrostriatal degeneration, can be detected by dopamenergic SPECT and can prove helpful in clinical differential diagnosis. A sensitivity of 83% and specificity of 100% has been reported for an abnormal scan with an autopsy diagnosis of DLBD.

Consensus criteria for diagnosing DLBD include ubiquitin immunohistochemistry for Lewy body identification and staging into three categories: brain stem predominant, limbic, or neocortical, depending on the numbers and distribution of Lewy bodies. The recently developed α-synuclein immunohistochemistry can visualize more Lewy bodies and is also better at indicating previously under recognized neurotic pathology, termed Lewy neurites. Use of antibodies to α-synuclein moves the diagnostic rating for many DLBD cases from brain stem and limbic groups into the neocortical group.

In most patients with DLBD, there are no genetic mutations in the α-synuclein or other Parkinson's disease-associated genes. Pathological up-regulation of normal, wild-type α-synuclein due to increased mRNA expression is a possible mechanism, or Lewy bodies may form because α-synuclein becomes insoluble or more able to aggregate. Another possibility is that α-synuclein is abnormally processed, for example, by a dysfunctional proteasome system and that toxic “proto fibrils” are therefore produced. Sequestering of these toxic fibrils into Lewy bodies could reflect an effort by the neurons to combat biological stress inside the cell, rather than their simply being neurodegenerative debris.

Target symptoms for the accurate diagnosis of DLBD can include extrapyramidal motor features, cognitive impairment, neuropsychiatric features (including hallucinations, depression, sleep disorder, and associated behavioral disturbances), or autonomic dysfunction.

### Multiple System Atrophy (MSA)

The present invention specifically provides methods, compositions, kits and arrays for the qualitative and quantitative assessment of dysregulated aggregate accumulation associated with MSA. MSA is a neurodegenerative disease marked by a combination of symptoms, affecting movement, cognition, autonomic and other body functions, hence the label “multiple system atrophy”. The cause of MSA is unknown. Symptoms of MSA vary in distribution of onset and severity from person to person. Because of this, the nomenclature initially included three distinct terms: Shy-Drager syndrome, striatonigral degeneration (SD), and olivopontocerebellar atrophy (OPCA).

In Shy-Drager syndrome, the most prominent symptoms are those involving the autonomic system; blood pressure, urinary function, and other functions not involving conscious control. Striatonigral degeneration causes Parkinsonism symptoms, such as slowed movements and rigidity, while OPCA principally affects balance, coordination and speech. The symptoms for MSA can also include orthostatic hypertension, male impotence, urinary difficulties, constipation, speech and swallowing difficulties, and blurred vision.

The initial diagnosis of MSA is usually made by carefully interviewing the patient and performing a physical examination. Several types of brain imaging, including computer tomography, scans, MRI, and positron emission tomography (PET), can be used as corroborative studies. An incomplete and relatively poor response to dopamine replacement therapy, such as Sinemet, may be a clue that the presentation of bradykinesia and rigidity (parkinsonism) is not due to PD. A characteristic involvement of multiple brain systems with prominent autonomic dysfunction is a defining feature of MSA and one that at autopsy confirms the diagnosis. Patients with MSA can have the presence of glial cytoplasmic inclusions in certain types of brain cells, as well. Prototypic Lewy bodies are not present in MSA. However, α-synuclein staining by immunohistochemistry is prominent. In comparison to Parkinson's, in addition to the poor response to Sinemet, there are a few other observations that are strongly suggested for MSA, such as postural instability, low blood pressure on standing (orthostatic hypotension) and high blood pressure when lying down, urinary difficulties, impotence, constipation, speech and swallowing difficulties out of proportion to slowness and rigidity.

Screening and assaying methods of the present disclosure upon a positive result can be used in combination with one or more alternative medications for treating MSA. Typically, the drugs that can be used to treat various symptoms of MSA become less effective as the disease progresses. Levodopa and dopamine agonists used to treat PD are sometimes effective for the slowness and rigidity of MSA. Orthostatic hypertension can be improved with cortisone, midodrine, or other drugs that raise blood pressure. Male impotence may be treated with penile implants or drugs. Incontinence may be treated with medication or catheterization. Constipation may improve with increased dietary fiber or laxatives.

### 2. Amyloidopathies

Amyloid precursor protein (APP) serves a variety of physiological functions, including modulation of synaptic function, facilitation of neuronal growth and survival, protection against oxidative stress, and surveillance against neuroactive compounds, toxins and pathogens. Two catabolic pathways have been described for processing of APP: the non-amyloidogenic and amyloidogenic cascade. The non-amyloidogenic pathway leads to formation of extracellular soluble N-terminal part of APP generated by α-secretase mediated cleavage. The amyloidogenic pathway results in the formation of the amyloid beta (Aβ) peptide by successive β-secretase and γ-secretase cleavages. Aβ is thought to be intrinsically unstructured, meaning that it cannot acquire a unique tertiary fold but rather populates a set of structures. The Aβ extracellular form is Aβ 1-40, while the intraneuronal Aβ corresponds to Aβ 1-42. Activation of the γ-secretase pathway in a pathological condition such as AD results in the accumulation of Aβ. This accumulation of Aβ results in diseases that are grouped under amyloidopathies.

The present disclosure provides methods, compositions, kits and arrays for the qualitative and quantitative assessment of dysregulated aggregate accumulation associated with amyloidopathies. For example, in some embodiments, the present invention provides a qualitative and quantitative assessment of the presence of amyloid beta proteins present in a sample of a subject who is suspected of having an amyloidopathy, such as Alzheimer's Disease or one that has been previously diagnosed and the assays of the present disclosure can be used to monitor progression of the disease or the effects of treatment. In certain embodiments, the amyloidopathy is Alzheimer's disease, vascular dementia, and/or cognitive impairment.

### 3. Tauopathies

Tauopathies are neurodegenerative disorders characterized by the presence of filamentous deposits, consisting of hyperphosphorylated tau protein, in neurons and glia. Abnormal tau phosphorylation and deposition in neurons and glial cells is one of the major features in tauopathies. The term tauopathy, was first used to describe a family with frontotemporal dementia (FTD) and abundant tau deposits. This term is now used to identify a group of diseases with widespread tau pathology in which tau protein accumulation appears to be directly associated with pathogenesis. Major neurodegenerative tauopathies includes sporadic and hereditary diseases characterized by filamentous tau deposits in brain and spinal cord.

In the majority of tauopathies, glial, and neuronal tau inclusions are the sole or predominant CNS lesions. Exemplary such tauopathies include amyotrophic lateral sclerosis (ALS), parkinsonism, argyrophilic grain dementia, diffuse neurofibrillary tangles with calcification, frontotemporal dementia linked to chromosome 17, corticobasal degeneration, Pick's disease, progressive supranuclear palsy, progressive subcortical gliosis, and tangle only dementia.

Additionally, tauopathies characterize a large group of diseases, disorders and conditions in which significant filaments and aggregates of tau protein are found. Exemplary such diseases, disorders, and conditions include sporadic and/or familial Alzheimer's Disease, amyotrophic lateral sclerosis/parkinsonism-dementia complex (ALS-FTDP), argyrophilic grain dementia, dementia pugilistica, diffuse neurofibrillary tangles with calcification, Down syndrome, frontotemporal dementia, parkinsonism linked to chromosome 17 (FTDP-17), Gerstmann-Straussler-Scheinker disease, Hallervorden-Spatz disease, inclusion body myositis, Creutzfeld-Jakob disease (CJD), multiple system atrophy, Niemann-Pick disease (NPC), Pick's disease, prion protein cerebral amyloid angiopathy, progressive supranuclear palsy (PSP), subacute sclerosing panencephalitis, tangle-predominant Alzheimer's disease, corticobasal degeneration, (CBD), myotonic dystrophy, non-guanamian motor neuron disease with neurofibrillary tangles, postencephalitic parkinsonism, prion protein cerebral amyloid angiopathy, progressive subcortical gliosis, subacute sclerosing panencephalitis, and tangle-only dementia.

Neurodegenerative diseases where tau aggregation pathology is found in conjunction with other abnormal protein lesions may be considered secondary tauopathies. Examples include AD and certain diseases where prion protein, Bri, or α-synuclein are aggregated. Although tau is probably not the initial pathological factor, tau aggregates contribute to the final degeneration.

Tau protein aggregation deposits can also be found in several other neurodegenerative diseases in which tau pathology is evident in conjunction with other abnormal protein lesions protein. Abundant cytoplasmic inclusions consisting of aggregated hyperphosphorylated protein tau are a characteristic pathological observation in several neurodegenerative disorders such as AD, Pick's disease, frontotemporal dementia, cortico-basal degeneration, and progressive supranuclear palsy.

The present disclosure provides methods, compositions, kits and arrays for the qualitative and quantitative assessment of dysregulated aggregate accumulation associated with tauopathies. For example, in some embodiments, the present invention provides a qualitative and quantitative assessment of the presence of aggregated tau proteins present in a sample of a subject who is suspected of having an tauopathy, such as sporadic and/or familial Alzheimer's Disease, amyotrophic lateral sclerosis/parkinsonism-dementia complex (ALS-FTDP), argyrophilic grain dementia, dementia pugilistica, diffuse neurofibrillary tangles with calcification, Down syndrome, frontotemporal dementia, parkinsonism linked to chromosome 17 (FTDP-17), Gerstmann-Straussler-Scheinker disease, Hallervorden-Spatz disease, inclusion body myositis, Creutzfeld-Jakob disease (CJD), multiple system atrophy, Niemann-Pick disease (NPC), Pick's disease, prion protein cerebral amyloid angiopathy, progressive supranuclear palsy (PSP), subacute sclerosing panencephalitis, tangle-predominant Alzheimer's disease, corticobasal degeneration, (CBD), myotonic dystrophy, non-guanamian motor neuron disease with neurofibrillary tangles, postencephalitic parkinsonism, prion protein cerebral amyloid angiopathy, progressive subcortical gliosis, subacute sclerosing panencephalitis, and tangle-only dementia, or one that has been previously diagnosed and the assays of the present disclosure can be used to monitor progression of the disease or the effects of treatment. In certain embodiments, the tauopathy is sporadic and/or familial Alzheimer's Disease, amyotrophic lateral sclerosis/parkinsonism-dementia complex (ALS-FTDP), argyrophilic grain dementia, dementia pugilistica, diffuse neurofibrillary tangles with calcification, Down syndrome, frontotemporal dementia, parkinsonism linked to chromosome 17 (FTDP-17), Gerstmann-Straussler-Scheinker disease, Hallervorden-Spatz disease, inclusion body myositis, Creutzfeld-Jakob disease (CJD), multiple system atrophy, Niemann-Pick disease (NPC), Pick's disease, prion protein cerebral amyloid angiopathy, progressive supranuclear palsy (PSP), subacute sclerosing panencephalitis, tangle-predominant Alzheimer's disease, corticobasal degeneration, (CBD), myotonic dystrophy, non-guanamian motor neuron disease with neurofibrillary tangles, postencephalitic parkinsonism, prion protein cerebral amyloid angiopathy, progressive subcortical gliosis, subacute sclerosing panencephalitis, and tangle-only dementia. In some embodiments, the cell expresses tau proteins. For example, in some embodiments, the present invention provides a qualitative and quantitative assessment of the presence of tau proteins present in a sample of a subject who is suspected of having a tauopathy, such as Alzheimer's Disease or one that has been previously diagnosed and the assays of the present disclosure can be used to monitor progression of the tauopathy disease or the effects of treatment.

### Alzheimer's Disease (AD)

AD is the leading cause of dementia and cognitive impairment in the elderly and a leading cause of death in developing nations after cardiovascular disease, cancer, and stroke. Up to 70% of cases of dementia are due to AD, with vascular disease being the second most common cause. The frequency of AD among 60-year-olds is approximately 1%. The incidence of AD doubles approximately every 5 years. Forsyth, Phys. Ther. 78:1325, 1998; Evans et al., JAMA 262:2551, 1989. Alzheimer's disease is the most common cause of dementia and the sixth leading cause of death in adults older than 65 years (Centers for Disease Control and Prevention, National Center for Health Statistics. National Vital Statistics System, Mortality 2018-2021 on CDC WONDER Online Database, released in 2021. Data from the Multiple Cause of Death Files, 2018-2021). The estimated total healthcare costs for the treatment of Alzheimer disease in 2020 is estimated at $305 billion, with the cost expected to increase to more than $1 trillion as the population ages. Wong W. Economic burden of Alzheimer disease and managed care considerations. Am J Manag Care. 2020 August; 26 (8 Suppl): S177-S183.

Alzheimers Disease is characterized by the deterioration of mental faculties (e.g., memory loss, confusion, loss of visual/spatial comprehension) and is associated with both amyloidopathies and tauopathies. The central role of the long form of amyloid 13-peptide, in particular Aβ (1-42), in Alzheimer's disease has been established through a variety of histopathological, genetic and biochemical studies. Specifically, it has been found that deposition in the brain of Aβ (1-42) is an early and innate feature of all forms of Alzheimer's disease. This occurs before a diagnosis of Alzheimer's disease is possible and before the deposition of the shorter primary form of Aβ, Aβ (1-40). Further implication of Aβ (1-42) in disease etiology comes from the observation that mutations in presenilin (γ-secretase) genes associated with early onset familial forms of Alzheimer's disease uniformly result in increased levels of Aβ (1-42). Additional mutations in APP raise total Aβ and in some cases raise Aβ (1-42) alone. Although the various APP mutations may influence the type, quantity, and location of Aβ deposited, it has been found that the predominant and initial species deposited in the brain parenchyma is long Aβ. In early deposits of Aβ, when most deposited protein is in the form of amorphous or diffuse plaques, virtually all of the Aβ is of the long form. These initial deposits of Aβ (1-42) then are able to seed the further deposition of both long and short forms of Aβ. In transgenic animals expressing Aβ deposits, these deposits were associated with elevated levels of Aβ (1-42), and the pattern of deposition is similar to that seen in human disease with Aβ (1-42) being deposited early followed by deposition of Aβ (1-40). Similar patterns and timing of deposition are seen in Down's Syndrome patients in which Aβ expression is elevated, and deposition is accelerated. The association of Alzheimer's Diseases with amyloid plaques means that Alzheimer's Disease is considered to be an amyloidopathy. Alzheimer's Disease is also associated with accumulation of tau aggregates and therefore is a tauopathy.

The present disclosure provides methods, compositions, kits and arrays for the qualitative and quantitative assessment of dysregulated aggregate accumulation associated with Alzheimer's Disease. For example, in some embodiments, the present invention provides a qualitative and quantitative assessment of the presence of amyloid beta, and/or tau proteins present in a sample of a subject who is suspected of having Alzheimer's Disease or one that has been previously diagnosed and the assays of the present disclosure can be used to monitor progression of the disease or the effects of treatment.

### Cognitive Impairment or Dementia

Cognitive impairment and dementia are highly prevalent neurological conditions associated with any of a variety of diseases, disorders, and/or conditions. Dementia is commonly defined as a progressive decline in cognitive function due to damage or disease in the body beyond what is expected from normal aging. Dementia is described as a loss of mental function, involving problems with memory, reasoning, attention, language, and problem solving. Higher level functions are typically affected first. Dementia interferes with a person's ability to function in normal daily life.

The cognitive impairment or dementia may stem from any etiology. Exemplary causes of cognitive impairment and dementia include neurodegenerative diseases, neurological diseases, psychiatric disorders, genetic diseases, infectious diseases, metabolic diseases, cardiovascular diseases, vascular diseases, aging, trauma, malnutrition, childhood diseases, chemotherapy, autoimmune diseases, ocular diseases, and inflammatory diseases. Particular diseases that are associated with cognitive impairment or dementia include, but are not limited to, atherosclerosis, stroke, cerebrovascular disease, vascular dementia, multi-infarct dementia, Parkinson's disease and Parkinson's disease dementia, Lewy body disease, Pick's disease, Alzheimer's disease, mild cognitive impairment, Huntington's disease, AIDS and AIDS-related dementia, brain neoplasms, brain lesions, epilepsy, multiple sclerosis, Down's syndrome, retinitis pigmentosa, wet and dry forms of age related macular degeneration, ocular hypertension, glaucoma, corneal dystrophies, Rett's syndrome, progressive supranuclear palsy, frontal lobe syndrome, schizophrenia, traumatic brain injury, post coronary artery by-pass graft surgery, cognitive impairment due to electroconvulsive shock therapy, cognitive impairment due to chemotherapy, cognitive impairment due to a history of drug abuse, attention deficit disorder (ADD), attention deficit hyperactivity disorder (ADHD), autism, dyslexia, depression, bipolar disorder, post-traumatic stress disorder, apathy, myasthenia gravis, cognitive impairment during waking hours due to sleep apnea, Tourette's syndrome, autoimmune vasculitis, systemic lupus erythematosus, polymyalgia rheumatica, hepatic conditions, metabolic diseases, Kufs' disease, adrenoleukodystrophy, metachromatic leukodystrophy, storage diseases, infectious vasculitis, syphillis, neurosyphillis, Lyme disease, complications from intracerebral hemorrhage, hypothyroidism, B12 deficiency, folic acid deficiency, niacin deficiency, thiamine deficiency, hydrocephalus, complications post anoxia, prion disease (Creutzfeldt-Jakob disease), Fragile X syndrome, phenylketonuria, malnutrition, neurofibromatosis, maple syrup urine disease, hypercalcemia, hypothyroidism, hypercalcemia, and hypoglycemia.

### B. Lysosomal Storage Diseases

Lysosomal storage diseases represent a set of disorders, diseases, and/or conditions characterized by a defect in lysosomal activity. In many embodiments, lysosomal storage diseases result from a decrease in the level or activity of one or more lysosomal enzymes. Lysosomal activity disruptions involved in lysosomal storage diseases may interfere, for example, with degradation of lipids, proteins or organelles by the lysosome, with proper trafficking of molecules into or out of the lysosome, and/or with lysosome-mediated signaling. Many lysosomal storage diseases are associated with accumulation of aggregates of one or more proteins in the lysosome (particularly of one or more proteins that is a substrate for a relevant lysosomal enzyme); such lysosomal storage diseases may be considered to be proteinopathies in accordance with certain embodiments of the invention. For example, in some embodiments, the present invention provides a qualitative and quantitative assessment of the presence of accumulation of aggregates of one or more proteins in the lysosome (particularly of one or more proteins that is a substrate for a relevant lysosomal enzyme) present in a sample of a subject who is suspected of having a lysosomal storage disease, or one that has been previously diagnosed and the assays of the present disclosure can be used to monitor progression of the disease or the effects of treatment.

Representative lysosomal storage diseases associated with accumulation of protein aggregates include, for example, Activator Deficiency/GM2 Gangliosidosis, Alpha-mannosidosis, Aspartylglucosaminuria, Cholesteryl ester storage disease, Chronic Hexosaminidase A Deficiency, Cystinosis, Danon disease, Fabry disease, Farber disease, Fucosidosis, Galactosialidosis, Gaucher Disease (e.g., Type I, Type II, Type III), GM1 gangliosidosis (e.g., Infantile, Late infantile/Juvenile, Adult/Chronic), I-Cell disease/Mucolipidosis II, Infantile Free Sialic Acid Storage Disease/ISSD, Juvenile Hexosaminidase A Deficiency, Krabbe disease (e.g., Infantile Onset, Late Onset), Metachromatic Leukodystrophy, Mucopolysaccharidoses disorders, Pseudo-Hurler polydystrophy/Mucolipidosis IIIA (e.g., MPSI Hurler Syndrome, MPSI Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC, Mucolipidosis type IV), Multiple sulfatase deficiency, Niemann-Pick Disease (e.g., Type A, Type B, Type C), Neuronal Ceroid Lipofuscinoses (e.g., CLN6 disease—Atypical Late Infantile, Late Onset mutant, Early Juvenile, Batten-Spielmeyer-Vogt/Juvenile NCL/CLN3 disease, Finnish Mutant Late Infantile CLN5, Jansky-Bielschowsky disease/Late infantile CLN2/TPP1 Disease, Kufs/Adult-onset NCL/CLN4 disease, Northern Epilepsy/mutant late infantile CLN8, Santavuori-Haltia/Infantile CLN1/PPT disease, Beta-mannosidosis), Pompe disease/Glycogen storage disease type II, Pycnodysostosis, Sandhoff disease/GM2 Gangliosidosis (e.g., Adult Onset, Infantile, Juvenile), Schindler disease, Salla disease/Sialic Acid Storage Disease, Tay-Sachs/GM2 gangliosidosis, Wolman disease, etc.

### C. Other Proteinopathies

Other proteinopathies may include, for example, inflammatory diseases, disorders, and/or conditions; proliferative diseases, disorders, and/or conditions; cardiovascular diseases, disorders, and/or conditions; immunologic diseases, disorders, and/or conditions; ocular diseases, disorders, and/or conditions; and/or mitochondrial diseases, disorders, and/or conditions.

The methods and assays, compositions and kits described herein can be effectively used to measure and quantify any accumulation of an aggregated protein associated with a proteinopathy as described herein. For example, in some embodiments, the present disclosure provides a qualitative and quantitative assessment of the presence of accumulation of protein aggregates present in a sample of a subject who is suspected of having a proteinopathy disease, or one that has been previously diagnosed, and the assays of the present disclosure can be used to monitor progression of the disease or assess the therapeutic benefit of treatment in a subject in need thereof, for example, of humans. Subjects who are especially served by the methods and assays of the present disclosure include human subjects, particularly humans who have suffered a neurodegenerative proteinopathy, for example, Alzheimer's disease (AD) (caused by Amyloid beta (Ab) peptide; Tau), Parkinson's disease (PD) (associated with α-synuclein), Huntington's disease (HD) (associated with Huntingtin with tandem glutamine repeats), amyotropic lateral sclerosis (ALS), ALS associated with Superoxide dismutase 1, Multiple tauopathies (associated with Tau protein (microtubule associated)), Spongiform encephalopathies (associated with prion proteins), Familial amyloidotic polyneuropathy (associated with transthyretin (mutant forms)) and, and chronic traumatic encephalopathy.

In various embodiments, the methods and assays, compositions and kits described herein can be effectively used to measure and quantify any accumulation of an aggregated protein present in pharmacological products. In certain embodiments, the methods and assays can be employed as described herein as described for subject fluid samples being substituted with samples of pharmacologic products in fluid form. Aggregates that may be present in protein products can range from small (dimers) to large assemblies (subvisible or even visible particles). They can be formed during production, storage, shipment or delivery to the patient. Numerous stresses (e.g., temperature fluctuations, light, shaking, surfaces, pH adjustments, etc.) can induce protein aggregation during each of these stages. Aggregation can occur because of exposure to air-liquid or liquid-solid interfaces, e.g., during mixing, during filling and shipping, during reconstitution of lyophilized products, or through contact with chromatography columns, pumps, pipes, vessels, filters, etc. Also, solution contact with ice during (adventitious or deliberate) freezing can cause aggregation. Moreover, protein aggregates may in some cases be induced by foreign particles, e.g., stainless steel and other particles from filling pumps, rubber particles from stoppers, salt crystals, glass particles generated during heating of containers for depyrogenation, and silicone oil droplets originating from siliconized syringes or stoppers. Protein aggregation may also be followed or induced by chemical degradations/modifications, e.g., oxidation. Current methods for detecting aggregation in pharmacologic products are rather cumbersome, expensive and require large samples. Some techniques presently used include microscopy, high performance size exclusion chromatography (SEC), SDS-PAGE and Capillary Electrophoresis-SDS, Dynamic light scattering (DLS), Analytical Ultracentrifugation (AUC), mostly used in sedimentation velocity mode (SV-AUC), Asymmetrical flow field-flow fractionation (AF4) with and without various detectors such as UV, refractive index and multi-angle laser light scattering (MALLS). None of these provides the totality of information available in a short timeframe from the microparticle capture assay described herein

Methods for performing detection and quantitative analysis of protein aggregates in pharmacologic products can include:
a. providing a capture substrate comprising a microparticle conjugated to a first capture moiety;
b. blocking the capture substrate non-specific binding sites;
c. incubating the capture substrate with a test pharmacologic product sample in liquid form suspected of having the aggregated protein for a period of time sufficient for the aggregated protein to specifically bind to the first capture moiety; wherein the first capture moiety binds specifically to the aggregate protein if present, thereby forming a capture complex on the capture substrate,
d. incubating the capture substrate with a second capture moiety that specifically binds to the aggregated protein, wherein the second capture moiety is coupled to a signalling moiety, and wherein the signaling moiety comprises a detectable label; and
e. determining the amount of detectable label present on the surface of the capture substrate.

Optionally, the amount of detectable label present on the surface of the capture substrate can be used to calculate the amount of detectable label present per unit of volume of test sample measured. Optionally, the amount of detectable label present on the surface of the capture substrate can be used to calculate the size and/or amount of the protein aggregate measured, per unit volume, or mass or other ratio specifically tested.

### Test Samples

In various embodiments, test samples are provided that may or may not contain aggregated proteins. Assays, compositions, kits, and systems of the present disclosure provide for the ability to measure the quantity and size of aggregates found in liquid solutions. In addition to the aggregated proteins found in the above described proteinopathies, the present disclosure is not so limited to these test samples. Other test samples may be routinely interrogated for the presence and amount of aggregated proteins. In some embodiments, test samples that can be interrogated using the present methods, kits, compositions and systems of the present disclosure include samples from plant, bacteria, viruses, yeast, invertebrates, vertebrates, e.g. mammals and fish, and preferably mammalian test samples, for example, laboratory mammalian test subjects, e.g. mice, rats, rabbits, hamsters, guinea pigs, ferrets and the like, and wild animals, for example, deer, coyotes, skunks, raccoons, squirrels etc., and farm production animals such as cows, cattle, pigs, goats and sheep as examples, but not an exhaustive list. Other mammalian test samples include non-human primates. The assays, methods, compositions, kits and systems of the present disclosure also contemplate interrogating human test samples, in particular human test samples that are provided by clinical specimens. In this regard, all mammalian test samples can include, but not limited to whole blood, umbilical cord blood, plasma, serum, saliva, urine, stool, colostrum, breast milk, bone marrow, lymph fluid, cerebral spinal fluid, peritoneal fluid, pleural fluid, joint fluid, vitreous fluid, and inflammatory fluid and tissue samples, for example, tissue biopsies that have been processed for the determination of aggregated proteins present in the tissue sample. Clearly, in some embodiments, the test samples are liquid samples comprising one or more proteins and the assays, methods, compositions, and systems are designed to determine the presence, quantity and size of aggregated proteins present in the test samples.

Mammalian test samples can be extracted from the test subject (mammal) using conventional techniques such as venipuncture, lumbar puncture (spinal tap), cisternal puncture, and ventricular puncture. Other specimen sample collection methods for obtaining test samples, such as urine, saliva and other specimens including tissue biopsies, are well known in the art

In other situations, aggregated proteins can be determined using the assays and methods of the present disclosure in laboratory specimens and reagents. In the laboratory, the basic biology of protein aggregation is studied using aggregation-prone proteins such as the disease-associated aggregated proteins implicated in the above described proteinopathies, but also any protein, like a housekeeping protein such as GAPDH. Many normal proteins used in the laboratory may aggregate under conditions of stress (such as heat or oxidation) or with time, and current laboratory methods for measuring protein aggregation are tedious and laborious and do not give an indication of the size of protein aggregates along with the amount or extent of aggregation. The described assays provide better, faster, determination of aggregate protein quantity and size than protein electrophoresis or ultracentrifugation, etc., for detecting protein aggregation in laboratory work. Specifically contemplated, is the detection of visible and sub-visable protein aggregates in solutions of pharmacological products. Within the context of protein aggregates in pharmacologic products, protein aggregation can be delineated as intrinsic and extrinsic. Intrinsic protein aggregation arises within the protein formulation during the synthesis and purification steps. Extrinsic aggregation, in contrast, results from the contacts of protein with external sources during processing, such as glass surfaces inside containers, stainless steel of bioprocessing equipment, or silicon oil droplets inside pre-filled syringes as well as extrinsic insults that occur after packaging such as temperature extremes, freezing and thawing, agitation, etc.

Other test samples from prion related diseases (chronic wasting disease (CWD)) in wildlife populations can be used as test samples used in the assays, methods, and systems described herein. For example, wild populations of buffalo, deer in various states of the United States that carry a potential threat of carry over of proteinopathies, for example prion related diseases into humans. For example, the Department of Natural Resources in Michigan monitors for CWD in the whitetail deer population, with the intent to reduce the potential threat of CWD transmission to hunters who consume the meat. Similar monitoring programs exist in other geographical locations where human populations consume high quantities of harvested wild game. The majority of human prion diseases are sporadic, but acquired disease can occur, as seen with variant Creutzfeldt-Jakob disease (vCJD) following consumption of bovine spongiform encephalopathy (BSE). With increasing rates of cervid chronic wasting disease (CWD), there is concern that a new form of human prion disease may arise. Currently, there is no evidence of transmission of CWD to humans, suggesting the presence of a strong species barrier; however, in vitro and in vivo studies on the zoonotic potential of CWD have yielded mixed results. The emergence of different CWD strains is also concerning, as different strains can have different abilities to cross species barriers. Given that venison consumption is common in areas where CWD rates are on the rise, increased rates of human exposure are inevitable. If CWD was to infect humans, it is unclear how it would present clinically; in vCJD, it was strain-typing of vCJD prions that proved the causal link to BSE. Therefore, the best way to screen for CWD in humans is to have thorough strain-typing of harvested cervids and human CJD cases using the assays and methods described herein, so that the investigators will be in a position to detect atypical strains or strain shifts within the human CJD population.

In some embodiments, the test sample can include pharmaceutical medicaments, pharmacological preparations, or products used to treat a disease or condition. In some embodiments, The Food and Drug Administration (US FDA) requires periodic monitoring of drug products that contain protein components for aggregation of these protein products, including medicaments and pharmaceutical preparations comprising at least one protein in solution. Test samples that are protein containing pharmaceutical products can include but are not limited to a liquid sample comprising: insulin, an antibody, a vaccine, a recombinant protein, a hormone, a cytokine, a chemokine, a growth factor, an enzyme, serum albumin, sera, plasma, erythropoietin (EPO), a receptor, a blood coagulation factor, an Fc fusion recombinant protein, and combinations thereof. These protein containing liquid medicaments, formulations and medicines (all used interchangeably) may develop protein aggregates over time, particularly when not stored under optimal conditions. If the protein in the preparation has aggregated, the protein aggregates could lose activity and when dosed in solution parenterally, for example, via intravenous injection or infusion, could potentially travel to and damage lung and other possible tissues. Pharmaceutical preparations of proteins need to be tested at intervals for protein aggregation which is one measure of the allowed shelf life (expiration) for the preparation. The assays, methods, compositions, kits and systems for measuring the amount and size of protein aggregates described herein are better, faster, more accurate and provide more information (size plus amount of aggregates) that is relevant to the pharmaceutical industry and the FDA.

### EXAMPLES

### Example 1—Methods

### Experimental Models and Subject Data

### Mouse Splenic B Cells

Whole spleens from C57BL/6 mice (The Jackson Laboratory) were first homogenized by gentle mechanical disruption with a syringe plunger and 70 μm mesh filter. B cells were subsequently isolated using the Miltenyi Pan B Cell Isolation Kit II (Miltenyi), in accordance with the manufacturer's instructions.

### Method Details

### Conjugation of Antibody to Aldehyde Sulfate Beads

Aldehyde sulfate beads (4 μm) (ThermoFisher) were resuspended in phosphate buffered saline (PBS) at a concentration of 2 μl bead stock/50 μl PBS (unless otherwise indicated). Anti-beta amyloid 1-42 monoclonal antibody (clone 12F4 mAb; Biolegend) was prepared at 0.8 μg/50 μl PBS (unless otherwise indicated). The solutions were combined in a 1.5 ml microcentrifuge tube, mixed gently by pipet, and incubated for 40 min at room temperature. To quench unoccupied binding sites on the beads, 1000 μl PBS was added to the solution, followed by 110 μl of 1M glycine. After a 15 min incubation at room temperature, 400 μl ice-cold blocking buffer (PBS/0.5% bovine serum albumin (BSA)/2 mM ethylenediaminetetraacetic acid (EDTA)) was added, and the solution was rotated for an additional 15 min. The sample was centrifuged at 4000 revolutions per minute (rpm) (Eppendorf tabletop centrifuge 5424R) for 3 min at 4° C. and the supernatant was removed by pipet. These conditions were utilized for all experiments to pellet the beads. To wash the beads and remove unbound 12F4 mAb in solution, the bead pellet was gently resuspended in 400 μl ice-cold blocking buffer and centrifuged. The beads were washed two additional times. After the final wash, the pellet was resuspended in 400 μl ice-cold blocking buffer. The resultant solution contained enough antibody-coated beads for 20 sample tests (20 μl/test at 0.04 μg 12F4 mAb/0.1 μl bead stock).

### Preparation of Aβ42 Multimers

Lyophilized monomeric Aβ42 (Anaspec) was prepared in assay buffer A (Anaspec) in Protein LoBind 1.5 ml microcentrifuge tubes (Eppendorf), according to the manufacturer's instructions. Fluorescently labeled monomeric Aβ42 (HiLyte Fluor 488 Aβ42; Anaspec) was reconstituted in 1% NH4OH and PBS as described by the manufacturer. To generate Aβ42 multimers, monomeric solutions (100 μl at 0.2125 μg/μl, unless otherwise indicated) were incubated at 1000 rpm/37° C. (ThermoMixer, Eppendorf) for the indicated period of time. The samples were then placed on ice for approximately 15 min and used for the assay, or frozen at −80° C. for future use. All Aβ42 monomer or multimer solutions were kept on ice for the duration of test sample preparation. Solutions containing Aβ42 monomers or multimers were kept in LoBind tubes for all stages of the assay and during storage.

Binding of Aβ42 to 12F4 mAb-Activated Aldehyde Sulfate Beads

Aβ42 test samples were prepared in 20 μl assay buffer A (unless otherwise indicated). Antibody-coated beads (in 20 μl ice-cold blocking buffer, unless otherwise indicated) were combined with the test samples, gently mixed by pipet, and incubated for 60 min at room temperature, with mixing at 30 min to re-disperse the beads in solution. Ice-cold blocking buffer (400 μl) was then added to each tube, followed by centrifugation and one wash. After the final spin, all supernatant was removed by pipet, less approximately 25 μl to maintain bead hydration.

### Detection of Bead-Captured Aβ42

Pelleted 12F4 mAb activated beads were resuspended in 50 μl biotinylated 12F4 mAb (Biolegend; prepared at 2.5 μg/ml in blocking buffer) and incubated for 60 min at room temperature, with mixing by pipet at 30 min. After addition of 400 μl ice-cold blocking buffer, the sample was centrifuged, washed, and centrifuged again, after which all supernatant was removed by pipet, less approximately 25 μl. The pellet was resuspended in 50 μl of streptavidin PE (eBioscience; prepared at 0.5 μg/ml in blocking buffer), mixed gently by pipet, and incubated for 20 min on ice, protected from light. Ice-cold blocking buffer (400 μl) was then added, and the sample was centrifuged, washed, centrifuged, and resuspended in 250 μl ice-cold blocking buffer for flow cytometry analysis.

### Flow Cytometry Acquisition and Analysis

All samples were acquired on a Fortessa Analyzer (Becton-Dickinson) at the low setting, yielding approximately 60-90 events/second for 0.1 μl bead stock/250 μl blocking buffer preparations. An FSC and SSC event threshold of 200 and voltage setting of 120 was utilized for all experiments. At least 4000 events of the gated bead population were acquired for each sample, and all analysis was conducted using Flowjo software (Becton-Dickinson). The gating scheme to identify the bead population for analysis, unless otherwise indicated, proceeded as: first gate on low FSC beads (FSC-A and SSC-A), second gate on single beads (FSC-A and FSC-W), third gate on single beads (SSC-A and SSC-W), and fourth gate on PE+ beads (PE and SSC-A), defined against the negative control.

### Microscopy Imaging of Aldehyde Sulfate Beads and B Cell Isolation

Aldehyde sulfate beads were prepared at a concentration of 1.0×10⁶ beads/100 μl ice-cold blocking buffer, aliquoted to a standard 48 well plate, and imaged by brightfield microscopy using a Lionheart FX automated live cell imager (Biotek). To stain beads directly for fluorescent imaging, 4 μl beads were combined with 4 μg donkey anti-rat AF594 (Life Technologies) in 50 μl PBS, followed by a 40 min incubation at room temperature. The sample was quenched with ice-cold blocking buffer, washed three times, and resuspended at a concentration of 1.0×10⁶ beads/100 μl ice-cold blocking buffer for imaging. B cells were isolated from C57BL/6 whole spleen single cell suspensions using the Miltenyi Pan B Cell Isolation Kit II (Miltenyi) and prepared at 1.0×10⁶ beads/100 μl ice-cold blocking buffer. Fluorescent beads and B cells were then mixed at a ratio of 50 μl:50 μl in a 48 well plate, incubated for 40 min, and imaged.

### Isolation of Aβ42 Multimers by Centrifugation

Aβ42 monomers (0.25 μg/μl assay buffer A) were placed on ice or incubated for 30 min or 60 min (2×100 μl/condition) at 1000 rpm/37° C. to form multimers. The multimer samples were transferred to thick-wall polycarbonate tubes (Beckman Coulter) and subjected to centrifugation at 20,000 rpm (15,456×g) for 30 min (Optima Max-TL Tabletop Ultracentrifuge (Beckman Coulter); TLA-100 fixed-angle rotor (Beckman Coulter)). Supernatant (100 μl) was removed carefully from the 30 min sample so as to not disturb the pellet and placed on ice. For the 60 min sample, supernatant was removed by pipet and the pellet was resuspended in 200 μl assay buffer

Following a second spin (20,000 rpm×30 min), all supernatant was removed carefully, and the pellet was resuspended in 40 μl assay buffer A. The protein concentration of each sample was determined by the bicinchoninic acid (BCA) method according to the manufacturer's directions (Pierce).

### Native Polyacrylamide Gel Electrophoresis (PAGE)

Samples were prepared 1:2 in 2× native sample buffer (62.5 mM Tris-HCl pH 6.8, 40% glycerol, 0.01% bromophenol blue) and resolved on 4-15% tris-glycine gradient gels (Bio-Rad). Sodium dodecyl sulfate (SDS) was excluded from the running buffer. The gels were stained with coomassie blue-based Imperial protein stain (Thermo Scientific) for 60 min at room temperature with gentle agitation, followed by several destain cycles in ultrapure water. Gel imaging and acquisition was completed on the chemidoc imaging system (Bio-Rad).

### Purification of Aβ42 Oligomers and Protofibrils by Size Exclusion Chromatography

To form Aβ42 oligomers and protofibrils, 15 μM Aβ42 monomers were vortexed for 30 seconds and then incubated at 4° C. for two weeks as previously described (Ryan et al., 2010). Samples were centrifuged at 14,000×g for 10 min at 4° C. to eliminate insoluble mature fibrils. Oligomers and protofibrils were purified by size exclusion chromatography as previously described with a minor modification (Esparza et al., 2016). Briefly, 0.25 ml of sample was injected onto an Enrich SEC 650 column (Bio-Rad) attached to an NGC Quest Chromatography system (Bio-Rad) and eluted with PBS containing filtered 0.05% BSA at a flow rate of 0.5 ml/min. A total of 19 1 ml elution fractions were collected and resolved by PAGE. Fractions 8 and 9 were pooled as protofibrils whereas fractions 11 and 12 were pooled as oligomers. The concentration of Aβ42 oligomers and protofibrils were determined by ELISA (Thermo Fisher Scientific). Protofibril and oligomer fractions were then analyzed by fluorescent microparticle immunocapture (“bead”) assay and flow cytometry as described.

### Quantification and Statistical Analysis

Prism software (version 8.1.1; GraphPad) was used to generate graphs and for statistical analyses. One-way ANOVA was performed for comparison of three or more groups to determine an overall difference, followed by a two-tailed t test between groups. Values of p<0.05 were considered significantly different. Coomassie stained gels were imaged on the chemidoc imaging system (Bio-Rad). ImageLab software (Bio-Rad) or photoshop (Adobe) was used to process and prepare images. All adjustments or transformations during image preparation were equivalently applied to the whole image. Microscopy images acquired using the Lionheart FX automated live cell imager (Biotek) were processed using image J software (National Institutes of Health). All adjustments or transformations during image preparation were equivalently applied to the whole image. Final figure layouts were completed using photoshop software (version 2018; Adobe).

### Results

### Assay Overview

The novel fluorescent microparticle immunocapture assay for protein aggregation described herein involves three steps (FIG. 1). Super active aldehyde sulfate beads are utilized as a microparticle platform upon which an epitope-specific, monoclonal capture antibody is bound by covalent interaction (FIG. 1A). After blocking with irrelevant protein, the loaded beads are incubated with a test solution containing the protein of interest, leading to antibody-protein interaction (FIG. 1B, binding epitope, green box) and subsequent protein capture and presentation on the bead surface. No unoccupied binding sites should exist on captured monomeric proteins due to antibody binding the single epitope, whereas unoccupied binding sites, or detection sites, will exist on captured protein multimers (FIG. 1B, green asterisks). A subsequent two-step incubation with the identical monoclonal antibody (biotinylated) and then streptavidin (SA) fluorophore allows detection of only protein multimers, while bound monomers go undetected (FIG. 1C). More unoccupied detection sites in larger multimers result in more antibody/SA fluorophore binding, and hence greater fluorescence. The fluorescence signal of individual beads is then quantitated by flow cytometer acquisition and analysis.

### Aldehyde Sulfate Bead Protein-Binding Characteristics and Flow Cytometry Acquisition

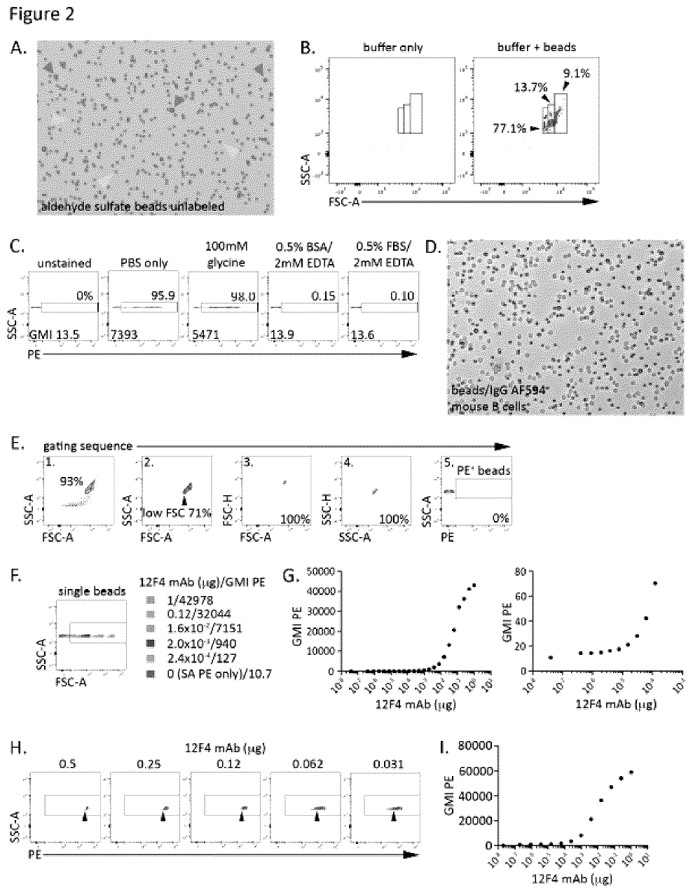

Although aldehyde sulfate bead use in research is well documented, the inventors validated the physical nature of the beads and the bead properties required for assay function, including capture antibody adsorption onto beads and detection by flow cytometry (Mendt et al., 2018; Suarez et al., 2017; Thery et al., 2006; Wahlgren et al., 2012). Aldehyde sulfate bead stock imaged by brightfield microscopy displayed a population that was largely uniform in diameter (yellow arrows), with a small percentage of larger diameter beads (turquoise arrows) (FIG. 2A). Bead populations could be visualized on a standard flow cytometry dot plot (see gated bead populations (right) vs. buffer alone (left)) using the physical parameters of size (FSC-A, X axis) and complexity (SSC-A Y axis) (FIG. 2B). The relative proportion of bead populations observed by microscopy was congruent with flow cytometry, with the large majority of beads (77.1%) in the lower FSC population. Glycine, followed by incubation and washes in a blocking agent such as BSA, is recommended by the manufacturer and reported in the literature as an effective block of non-specific protein adsorption to beads (Thery et al., 2006). To demonstrate this, the inventors first incubated beads with buffers of predicted variable blocking efficiencies, followed by incubation with fluorescent antibody. Flow cytometry analysis and quantitation of the geometric mean intensity of PE (GMI PE) indicated that beads incubated in 100 mM glycine/PBS were blocked to some extent (GMI PE 5471 vs. 7393 for PBS alone), however addition of an irrelevant protein such as BSA or FBS provided a complete block of antibody binding (GMI PE 13.9 and 13.6 vs. 13.5 for unstained beads) (FIG. 2C). As an additional confirmatory step, the inventors first labeled beads with fluorescent antibody, blocked in glycine/BSA, mixed beads with sorted B cells, and imaged the solution by brightfield microscopy. The inventors observed fluorescently labeled beads of the expected size (4 μm bead vs. 8-10 μm B cell) that were in solution unbound to the protein-rich surface of B cells (FIG. 2D). Therefore, the blocking conditions of 100 mM glycine PBS followed by 0.5% BSA/2 mM EDTA PBS were utilized for the duration of the experiments.

The inventors next determined 12F4 mAb-to-bead binding properties, with the goal to identify the amount of antibody that would provide near maximum binding potential to amyloid peptides in solution without reaching the coating saturation of the beads. Adsorption onto polystyrene substrates can result in protein layering at high concentrations, leading to unstable outer layers that can have an enhanced potential to dissociate from the substrate (Butler, 2000; Volger, 2012). Titrated biotinylated-12F4 mAb (range of 4×10⁻⁷ μg-1 μg/50 μl) was incubated with 1 μl AS bead stock/50 μl (total incubation volume 100 μl), followed by block, wash, SA PE incubation, and flow cytometry acquisition and analysis. Application of a standard flow cytometry gating scheme to beads that received SA PE only (no 12F4 mAb) allowed the identification of single PE+ beads (FIG. 2E). Analysis of the 12F4 mAb titrated samples utilizing this scheme indicated that bead PE fluorescence increased with increasing 12F4 mAb concentration, up to 0.25 μg, whereupon the slope of the curve started to flatten (FIGS. 2F; 2G (left graph all titrated samples, right graph most dilute samples only)). A comparison of the dot plots from beads in the antibody range 0.031 μg to 0.5 μg showed a distinct sub-saturated fluorescence tail at levels below 0.25 μg, not present at 0.5 μg (FIG. 2H, black arrowhead). The inventors then conducted a 12F4 mAb titration using 10-fold less beads (0.1 μl bead stock/100 μl total volume) and observed a similar curve based on the 12F4 mAb: bead stock ratio (FIG. 2I), thus indicating that bead adsorption at 0.4 μg 12F4 mAb/1.0 μl AS bead stock was sufficient to coat the beads to a high degree but remain below bead saturation.

### Evaluation of 12F4 mAb Binding to Aβ42, Optimal Analyte Concentration, and Assay Sensitivity

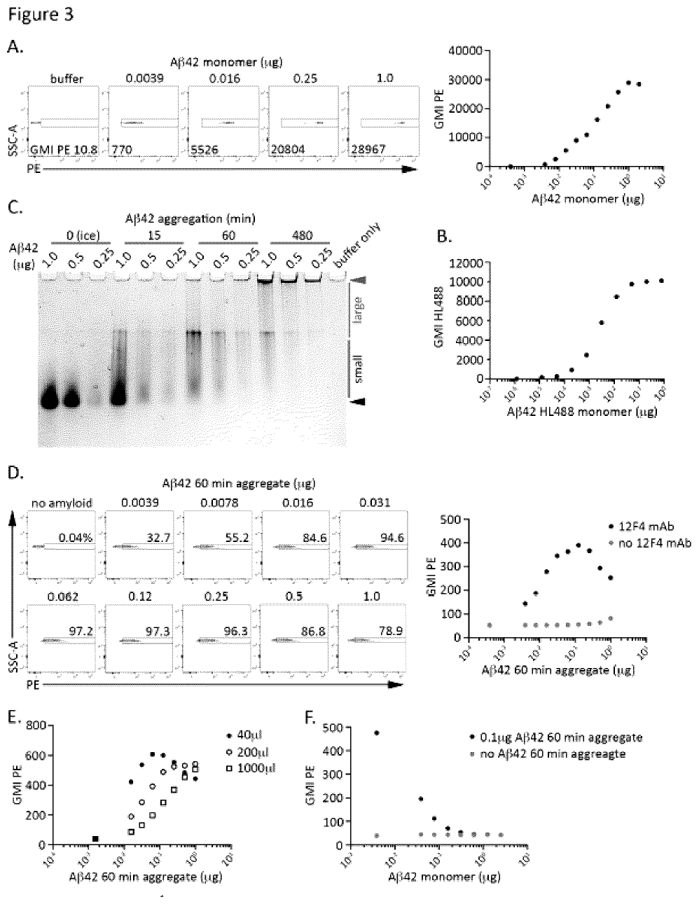

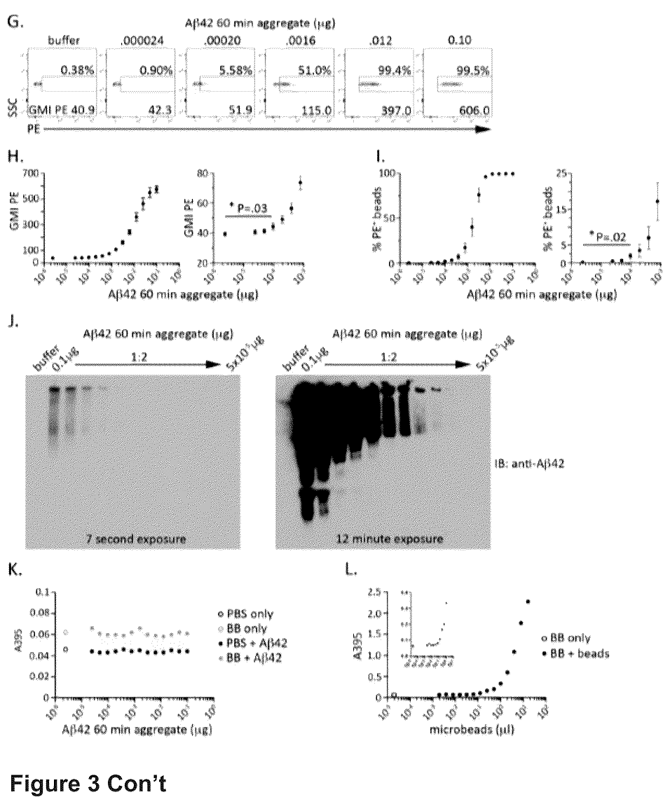

The results to this point suggested that aldehyde sulfate beads constitute a suitable platform for immunocapture of a protein target. Although use of 12F4 mAb is reported in the literature, the inventors wanted to confirm that it could bind Aβ42 specifically under conditions dictated by the assay. The inventors first incubated titrated monomeric Aβ42 directly with reactive beads to allow adsorption, followed by wash and then block. The samples were then incubated with an equivalent amount of biotinylated 12F4, followed by wash, SA PE incubation, wash, and flow cytometry analysis. As expected, increased amounts of Aβ42 resulted in higher bead fluorescence (FIG. 3A), indicating that 12F4 mAb could bind Aβ42. A similar curve (based on Aβ42 (μg): bead stock ratio (μl); plateau ˜0.1 μg:0.1 μl) was observed when fluorescently conjugated Aβ42 (HL488 Aβ42) was adsorbed directly onto beads (FIG. 3B), providing additional support that the fluorescence measured following 12F4 mAb-mediated detection in FIG. 3A was in fact due to 12F4 mAb binding to Aβ42. The inventors then used established experimental conditions, heat and agitation, to induce aggregation of monomeric Aβ42. Native polyacrylamide gel electrophoresis (PAGE) of monomer solutions incubated for 15, 60, or 480 min at 37° C./1000 rpm illustrated the comparative aggregation state of Aβ42 (FIG. 3C). Monomeric Aβ42 was largely restricted to a single band; Aβ42 incubated for 15 and 60 min showed evidence of increasing aggregation into larger multimeric forms whereas Aβ42 incubated for 480 min resulted in very large species that failed to enter the gel. The 60 min solution appeared to have the widest spectrum of Aβ42 multimeric species, small ranging to large, and thus was utilized for subsequent experiments. The inventors first evaluated assay parameters that could result in a hooking effect, a commonly observed phenomenon in research and clinical ELISA-based assays where analyte concentrations above the assay hook point result in reduced signal intensity (Dodig, 2009; Erickson et al., 2016; Jassam et al., 2006). Titrated 60 min Aβ42 material was incubated with beads lacking capture antibody, or with 12F4 mAb-loaded beads, followed by detection and flow cytometry analysis. The inventors observed an increase in both the percentage of PE+ beads and GMI PE as Aβ42 amount increased (range 0.0039 μg to approximately 0.1 μg); however, there was a marked decrease in bead fluorescence at higher levels of Aβ42 (FIG. 3D). This indicated that the hook point was around 0.1 μg Aβ42/100 μl buffer. Control beads without capture antibody showed no to minimal fluorescence above background, indicating that 12F4 mAb mediated-Aβ42 capture was required for detection of antibody binding, and thus provided further confirmatory evidence for efficient bead blocking and antibody specificity. Increasing the reaction volume from 40 μl to 200 μl to 1000 μl, thereby decreasing the analyte concentration, resulted in a shift of the fluorescence curve to the right (FIG. 3E). This provided further evidence that Aβ42 binding, and detection was dependent on Aβ42 concentration, and as a result the inventors set 0.1 μg Aβ42/40 μl-100 μl as an upper concentration limit to stay within the working detection range of the assay.

Using these assay parameters as a guide, the inventors performed a competition experiment to determine if Aβ42 aggregate-to-bead binding could be blocked if incubation occurred in the presence of monomeric Aβ42. Equivalent 0.1 μg 60 min Aβ42 aggregate samples were first combined with titrated monomer and then incubated with 12F4 mAb-loaded beads under standard conditions (60 min, room temperature). Detection and flow cytometry acquisition proceeded by standard methods as well. The data revealed that the inventors could effectively compete 60 min Aβ42 aggregate binding with monomer levels greater than 1.0 μg, and that monomer at all tested amounts incubated without Aβ42 aggregates showed no appreciable fluorescence above background (FIG. 3F). This indicated that the 12F4-Aβ42 interaction is specific, and that only higher order Aβ42 multimers, not monomers, are detected by the assay.

Having established the bead-12F4 mAb conjugation amount, the effective assay blocking conditions, and the assay hook concentration, and having confirmed antibody specificity, the inventors then evaluated assay reproducibility and sensitivity. Titrated solutions from the identical stock of Aβ42 aggregate were prepared every other day over five days and analyzed. Assay parameters and methods were stringently consistent. The results indicated that bead GMI PE and percentage PE+ beads were reproducible, as determined by the mean values across days (FIGS. 3G, 3H, and 3I). Notably, detection of aggregates above background was significant at sub-nanogram levels, indicating sensitivity on the scale of ELISA-based methods and immunoblot (FIGS. 3H and 3I, asterisks).

### Bead Assay Detection of Aβ42 Oligomers and Protofibrils

Multiple studies indicate the form of an aggregate, whether small order multimers (oligomers), intermediate-sized proto-fibrils, or large fibrils, may convey unique functional and pathophysiological consequences (Chen et al., 2017; Haass et al., 2007). To test the ability of the bead assay to detect Aβ42 oligomers or proto-fibrils specifically, the inventors utilized established protocols to generate both species from Aβ42 monomers (Esparza et al., 2016; Ryan et al., 2010). Following size exclusion chromatography, the collected samples were resolved by PAGE, and as expected the inventors observed fractions enriched for either protofibrils (slower migration; fractions 8,9) or oligomers (faster migration; fractions 11,12) (FIG. 4A). The indicated fractions were then pooled, Aβ42 concentration was determined by BCA method, and the samples were titrated and evaluated by the bead assay. The results demonstrated that both oligomers (FIG. 4B) and protofibrils (FIG. 4C) could be detected above no amyloid control at sub-nanogram levels, in line with what was observed above for the Aβ42 60 min aggregate sample.

### Bead Assay Characterization of Aβ42 Multimer Size

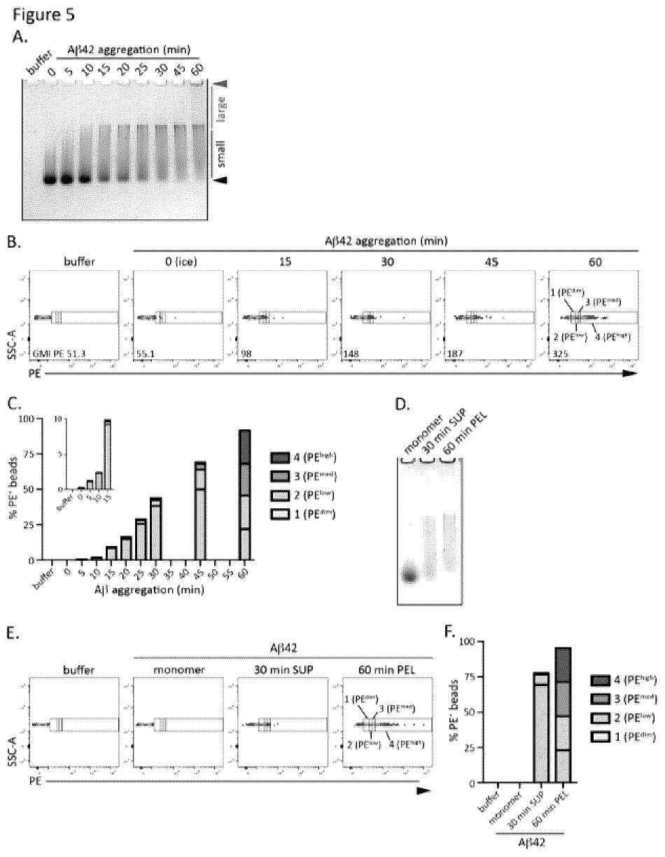

Current methods employed to study protein aggregation are qualitative to the presence of aggregation, but do not measure aggregate size. To address the utility of the bead assay for measurement of this parameter, solutions of monomeric Aβ42 were incubated with heat and agitation (37° C./1000 rpm) for different periods of time. This standardized method, common for assays such as ThT, produces progressively higher order multimers. Samples were harvested every five minutes for the first 30 min, and then at 45 min and 60 min. Samples from each time point were resolved by native PAGE and displayed progressive transformation of majority monomeric Aβ42 at no incubation (0 min; black arrow) to a mix of smaller (black line) and larger (red line) multimers at 60 min, with some protein species being of such large size that they fail to migrate (red arrow) (FIG. 5A). An equivalent amount of protein from each time point was then analyzed by the bead assay, with the PE positive bead population first established using the buffer only sample (no Aβ42). This population was then divided into four proportionally equivalent populations (quadrants) using the sample that displayed the highest overall fluorescence intensity (FIG. 5B, 60 min aggregation time). Given that only multimers, and not monomers, can produce fluorescent beads above background levels, the inventors could determine not only the percentage of beads that displayed positive fluorescence (multimer detection), but also the proportion of beads in each of the four populations based on relative bead PE intensity (1 PE^{dim}, 2 PE^{low}, 3 PE^{med}, 4 PE^{high}). Applied to all time points, the gating revealed that longer aggregation time resulted not only in more PE positive beads, but also proportionally brighter beads (FIG. 5C). In particular, whereas between 45 and 60 min, the increase in the amount of positive beads was small, the increase in PE^{high} beads was dramatic, from essentially no PE^{high} beads at 45 min to a quarter of all the beads at 60 min. This was accompanied by a marked decrease in the fraction of PE^{dim} beads. Moreover, the increase in positive beads with aggregation time corresponded with our observed increase in higher order Aβ42 multimer species (FIG. 5A). Additionally, the most notable shift to brighter beads, at 45 min and 60 min, is in line with the formation of the highest order multimers at these time points as confirmed by native gel. These results demonstrate that the microparticle immunocapture assay reflects multimer size independently of multimer amount.

To further test this result indicating that larger aggregates produce brighter beads, the inventors utilized centrifugation to isolate Aβ42 multimers based on sedimentation velocity (Mok et al., 2006; Stine et al., 2003). Multimeric species were first generated by incubation (37° C./1000 rpm) for 30 or 60 min, followed by centrifugation to isolate comparatively larger (pelleted, 60 min PEL) and smaller (supernatant, 30 min SUP) protein species. Samples resolved by native PAGE exhibited the expected shift in migration when compared to Aβ42 monomer, with the 60 min PEL displaying reduced migration compared to 30 min SUP (FIG. 5D). Application of the gating scheme described above for bead assay analysis revealed that whereas the amount of PE positive beads in the 60 min PEL sample was only minimally increased compared to the 30 min SUP, the fraction of PE^{med} to PE^{high} beads was markedly increased (FIGS. 5E and 5F), and the fraction of PE^{dim} beads was markedly decreased. These results provide additional evidence that increased Aβ42 size is reflected in increased bead fluorescence. In total, these data suggest that the bead assay can measure not only the degree of protein multimerization but also distinguish multimers by size.

### Detection of Alpha-Synuclein (αS) Protein Aggregates

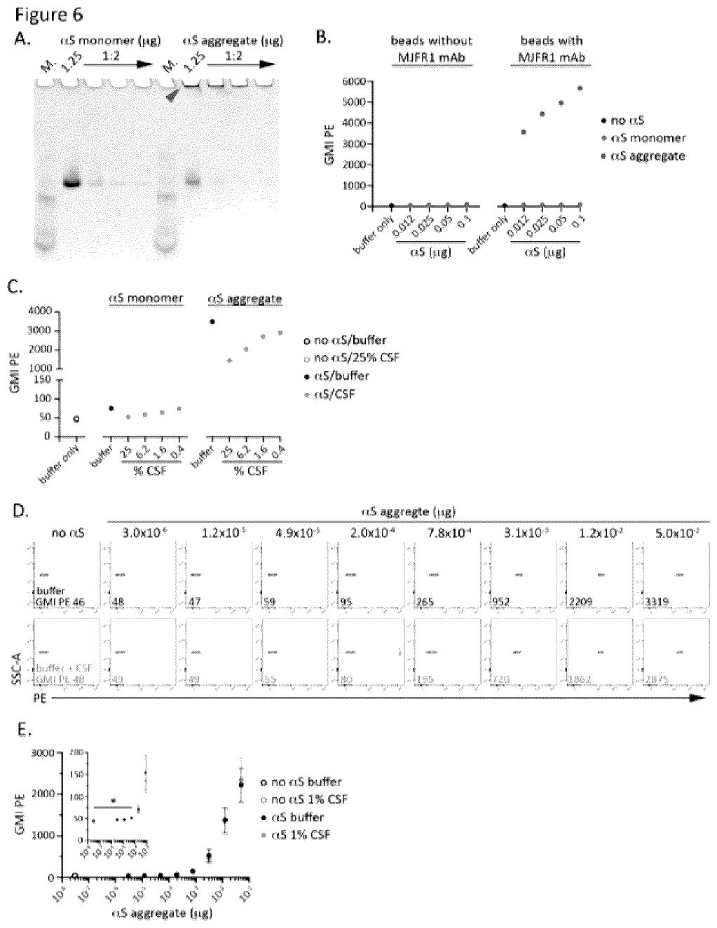

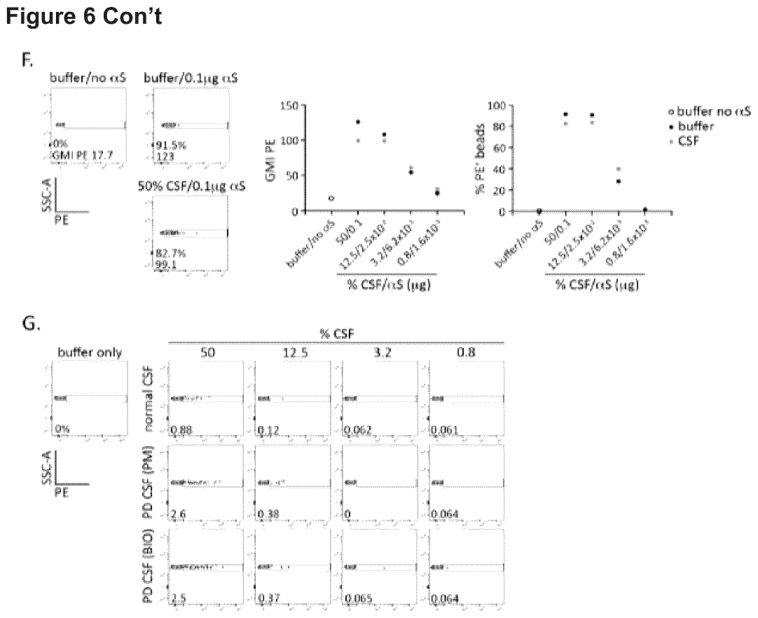

To test the versatility of the microparticle immunocapture platform the inventors examined aggregation-prone alpha-synuclein (αS). This protein is involved in several neurodegenerative diseases, including Parkinson's disease, in which the multimerization of monomeric alpha-synuclein corresponds with disease progression in the brain and thus has been a target of high interest for quantitative assay development. The inventors first acquired commercially available human wild type monomeric αS and induced aggregation with heat and agitation according to the manufacturer's instructions. Resolved αS monomer samples by native PAGE revealed a predominate single band (FIG. 6A, first four sample lanes). The presence of a higher order species (red arrow), along with a reduction in amount of the faster migrating monomer species, indicated that the sample had undergone aggregation (last four lanes). To this point, the proof-of-concept approach for the bead assay had utilized Aβ42, so the inventors next validated the critical bead assay characteristics of target specificity and aggregate only detection using αS. Beads activated with or without the anti-αS mAb MJFR1 were incubated with titrated αS monomer or αS aggregate samples and subsequently subjected to a similar block, wash, detection (here PE conjugated MJFR1), and flow acquisition protocol as utilized for the bead assays involving Aβ42. Prior experiments (data not shown) had revealed that the optimal mAb: bead conjugation ratio and αS amount were similar to those established for Aβ42, conditions that allowed for maximal bead intensity while staying below the hook point for the assay. Incubation of either αS monomer or αS aggregate with non-MJR1 activated beads resulted in bead fluorescence that was near background levels (FIG. 6B, left side of graph). The same was true for MJFR1-activated beads incubated with αS monomer. However, incubation of activated beads with αS aggregate samples produced markedly brighter beads whose fluorescence intensity increased with increasing levels of αS (FIG. 6B, right side of graph). These results indicated that the MJFR1-conjugated bead assay platform was specific for aggregated αS only and could measure the amount of aggregated αS.

### Detection of Alpha-Synuclein (αS) Protein Aggregates in Human Cerebrospinal Fluid

There is much interest in the detection and measurement of protein aggregates in biological specimens, as increased aggregation state is associated with cellular and protein dysfunction. However, although simple in concept, the ability to detect protein species in biological matricies is often compromised to some degree by yet uncharacterized factors present in the matrix that do not exist in standard, controlled laboratory buffers and solutions, for which reason it is often recommended to test biological fluids at 1% concentration (DeForge et al., 2007; Lachno et al., 2015; Mollenhauer et al., 2008). The inventors therefore wanted to test the utility of the bead assay in detection of aggregated αS in human cerebrospinal fluid specimens. Equivalent amounts of monomeric αS prepared in buffer alone or in buffer with titrated CSF, incubated with MJFR1-activated beads, and subsequently analyzed by flow cytometry produced bead intensities with similar background (FIG. 6C, left side of graph). With aggregated αS, the inventors observed a progressive decrease in bead fluorescence compared to buffer alone as the amount of CSF increased (0.4% to 25% CSF), however even at 25% the signal intensity was well above background (GMI PE 47.6 vs. 1452). These results showed that although the inventors did observe a matrix influence on αS detection, a result that was not unexpected based on previously published reports, the inventors could detect aggregated αS, and not monomeric αS, in a physiologically and pathologically relevant biological material. Building from these results, the inventors then determined the limits of detection of aggregated αS in CSF. Titrated αS aggregate was prepared in either buffer or buffer+1% CSF and analyzed by the bead assay. Flow cytometry dot plots show a comparable bead fluorescence profile (spread) and PE intensity (GMI) for buffer (top row) and buffer/CSF (bottom row) samples (FIG. 6D; GMI in lower left of plot). Data from four independent assays indicated equivalent fluorescence values for the two buffer conditions across all tested samples (FIG. 6E, inset graph low αS levels only). Further, the inventors observed a significant difference in PE intensity above background for both conditions at approximately 4.9×10⁻⁵ μg αS (GMI PE: buffer 44.2 vs. 51.9, buffer/CSF 45.2 vs. 51.1). Thus, the microparticle immunocapture assay represents a platform that can be readily adapted to detect other species of protein aggregates, in this case αS, and can do so in a biological fluid (CSF).

To this point, validation and application of the assay involved CSF from normal human donors spiked with aggregated αS prepared in vitro under experimentally controlled conditions. Given the potential significance of establishing the microparticle platform as a methodological tool for aggregate quantitation in diseased individuals, the inventors next wanted to assess how the assay would perform on CSF collected from Parkinson's disease (PD) patients. The inventor's analysis of aggregated αS added to normal CSF, and then titrated in buffer, indicated that optimal detection occurred when the samples in CSF were less dilute (left and right graphs, orange dots), however bead fluorescence was most near buffer conditions (black dots) as the dilution increased (FIG. 6F). This was in line with our results using equivalent αS amount in titrated CSF (see FIG. 6C). Using similar parameters to analyze PD CSF samples for the presence of aggregated αS, the inventors observed a distinct population of PE+ beads in both evaluated PD CSF samples that decreased with sample titration (FIG. 6G, rows two and three, gated population). Notably, the populations displayed a relatively broad fluorescence pattern (compare to FIG. 6F dot plot, bottom row), indicating the existence of higher order aggregates, and as expected were approximately three to four fold greater in number than what was detected in normal CSF (column one, 2.6 and 2.5% vs. 0.88% at 50% CSF; column two, 0.38 and 0.37 vs. 0.12 at 12.5% CSF) (Shahnawaz et al., 2020). These results indicate that the bead assay can not only detect naturally generated aggregated «S in CSF, but also suggest assay utility for comparative quantitative analyses across multiple patient samples.

### Discussion

Maintenance of protein homeostasis (proteostasis) is essential for cellular and organismal health. The presence of mutant or otherwise aggregation-prone proteins, and/or the dysfunction of pathways to eliminate damaged or disordered proteins, can result in protein accumulation, aggregate formation, and ultimately cell death. Given the strong experimental and observational evidence connecting protein aggregation with neurodegenerative and other diseases, methodologies to assess protein aggregation have been developed and relied upon by basic science and clinical investigators. However, there are substantial deficiencies in the repertoire of the tests presently available that hinder a more complete analysis of protein aggregation. In particular, measurement of multimer size has been lacking. Herein the inventors present a novel bead fluorescence assay to detect protein aggregates that uses highly quantitative flow cytometry analysis to discriminate protein monomers from multimeric species and characterize the degree of protein multimerization.

The presently exemplified microparticle immunocapture platform for assaying the presence of aggregated Aβ42 protein, incorporates a monoclonal capture antibody (anti-Aβ42 mAb clone 12F4) adsorbed onto 4 μm super active aldehyde sulfate beads, followed by sample incubation and capture, detection antibody incubation, and flow cytometry analysis. In theory, target protein binding to the capture mAb will saturate all available antibody binding sites on a monomeric target and thus prohibit any subsequent binding of a detection antibody fashioned from a mAb identical to the capture mAb. Multimer capture will occur by the same process, however sites will remain unoccupied and thus open to binding by the detection mAb, with the number of unoccupied sites proportional to the number of individual units that comprise the multimer. The results provided herein demonstrate that there was no appreciable binding of detection antibody to monomeric Aβ42 captured by 12F4 loaded beads, whereas aggregated Aβ42 was detected (FIGS. 3D and 3F). Importantly, the capture mAb:Aβ42 aggregate interaction was specific, as the inventors effectively competed bead-captured aggregate with titrated Aβ42 monomer. A mAb targeting any protein of interest could be substituted during the bead activation step, thus providing high adaptability to meet experimental demands. An additional advantageous feature is the short duration required to complete the assay. Capture antibody adsorption followed by effective blocking (0.5% BSA/2 mM EDTA) to eliminate downstream non-specific binding results in the generation of a highly specific bead: capture mAb platform in under 90 min. Subsequent steps of sample and detection mAb incubations, including washes, were completed in under four hours, with small time variations due to sample number. Flow cytometry acquisition required approximately 90 seconds/sample, and the single fluorescent parameter analysis using Flowjo software was inherently very streamlined. Data could thus be generated for a sample set, from start to finish, in less than one working day.

The inventors showed by native PAGE that larger Aβ42 protein species were formed as aggregation time increased, concomitant with a decrease in monomer species (FIG. 5A). Bead incubation with an equivalent amount of Aβ42 from each aggregation time point generated a progressive increase in bead fluorescence intensity and percentage of PE+ beads (FIGS. 5B, 5C, 5D), in line with the gel results provided herein. At even the earliest time points (5, 10, 15 min), the inventors were able to distinguish fluorescent beads above background (buffer alone), coincident with an increase in Aβ42 size and reduction in Aβ42 monomer observed by gel. These results indicate that the assay detects not only larger Aβ42 aggregates, but much smaller protein species that are formed in the early stages of multimerization. Comparatively small and large Aβ42 multimers isolated by centrifugation exhibited the expected relative fluorescence intensities, providing additional support that the bead assay captures and distinguishes small and large Aβ42 multimers (FIG. 5G).

A limitation of several commonly utilized assays, including ThT and plate-based ELISA (El-Agnaf et al., 2000; El-Agnaf et al., 2006; Gade et al., 2017), is that signal intensity reflects only the overall degree of aggregation for a sample, while differences in size of individual aggregates that comprise the total are not detected or quantified. Although precise, accurate, and robust quantitation of total aggregation is informative and valuable, there is mounting evidence that aggregate size, small oligomer vs. proto-fibril vs. fibril, has biological significance. Size analysis by immunoblot, EM, or gel filtration chromatography (GFC) can be informative, but application may be limited due to practical considerations for immunoblot and EM, and the limited size range for GFC. The inventors suggest that the inherent characteristics of the bead platform provide substantial advantages. A bead that has captured in proportion more larger aggregates will fluoresce more than a bead with smaller aggregates due to a greater number of available detection mAb binding epitopes. Thus, sample solutions that have a greater proportion of large aggregates to small aggregates will generate beads that display higher fluorescence. This is clearly shown by flow cytometry dot plot analysis. In FIG. 5, four equivalent gates (each 23%+/−1%) were constructed for the fluorescence profile of the sample that displayed the highest overall PE intensity (60 min aggregation) and then applied to all samples. Although the total number of PE+ beads varied (increasing in number with aggregation time, see FIG. 5D), it would be expected that if aggregate size had no influence on bead fluorescence, then the proportion of beads in each of the four populations would be 23% across all samples. This was not the case, as only at the later time points (30, 45, 60 min) were bead populations that are mid to high fluorescence observed (see FIG. 5F). Moreover, PE high beads were restricted primarily to the 60 min aggregate sample, which correlates with the presence of larger aggregates that failed to migrate during native PAGE (see FIG. 5A, red arrow). A similar result occurred with Aβ42 multimers isolated by centrifugation. Only the 60 min PEL sample (comparatively larger multimers, FIG. 5G) displayed a significant proportion of mid and high PE+ beads (FIG. 5I). These results highlight a distinct advantage of the bead platform over plate ELISA, in which the aggregation state of individual particles is averaged-solutions comprised of many small aggregates will appear equivalent to those with few, but larger aggregates. With beads, aggregate size is discernable. Many small multimers will generate lots of less bright beads whereas a few large aggregates will generate less abundant, but very bright beads. Thus, the relative proportions of protein multimers based on size-small vs. medium vs. large-will be reflected in fluorescence intensity. This type of analysis can be utilized with any sample set, and population gates can be set based on criteria established by the investigator. Further, flow cytometry allows equal application of these gates across all samples, thus ensuring that characterization of aggregate size based on bead fluorescence will be unbiased.

The commercial availability of microbeads harboring a wide range of physical and chemical properties present opportunities for additional optimization. The inventors utilized aldehyde sulfate beads because of the aforementioned functional advantages and literature record, and numerous experiments were completed to establish mAb: bead ratios and incubation conditions that offered a suitable balance to minimize reagent requirements, enhance protein capture efficiency, generate maximal fluorescence signal intensity, and optimize flow cytometry acquisition parameters. It also became apparent in the experiments conducted and described herein, that protein (analyte) amounts above a certain concentration resulted in decreased bead fluorescence as the amount of protein increased. The fluorescence intensity curves display characteristics of the hook effect, a common feature to immunological assays that is attributed to excess analyte or antibody in solution (Dodig, 2009; Erickson et al., 2016; Jassam et al., 2006). The observed hook point was very consistent and could be manipulated with changes to protein concentration. Identification of the hook point can be achieved by a simple dilution and would need to be completed for any tested protein.

The inventors propose that this novel microparticle immunocapture assay for quantitating protein aggregation is well suited for use in the research lab and in the analysis of clinical fluid and/or tissue samples in human therapies and diagnosis. Sample manipulation during the course of the assay is limited and thus the potential introduction of artifacts is lessened. It is specific (antibody-mediated detection), rapid (same day results), quantitative (flow cytometry readout) and readily adaptable to detect other species. Further, and perhaps most notably, aggregates can be distinguished based on size. Given these attributes, the inventors suggest that the assay possesses distinct advantages beyond the traditional methods of evaluation available up to now.

1. A method for determining the presence and at least one of, the size and amount of an aggregated protein in a test sample, the method comprising:
a. providing a capture substrate comprising a microparticle conjugated to a first capture moiety;
b. blocking the capture substrate non-specific binding sites;
c. incubating the capture substrate with a test sample suspected of having the aggregated protein for a period of time sufficient for the aggregated protein to specifically bind to the first capture moiety; wherein the first capture moiety binds specifically to the aggregate protein if present, thereby forming an capture complex on the capture substrate,
d. incubating the capture substrate with a second capture moiety that specifically binds to the aggregated protein, wherein the second capture moiety is coupled to a signalling moiety, and wherein the signaling moiety comprises a detectable label; and
e. determining the amount of detectable label present on the surface of the capture substrate.

2. The method of claim 1, wherein the aggregated protein comprises native, variant, mutant or posttranslationally modified forms of: α-synuclein, tau, amyloid beta (Aβ42 and Aβ40), SOD1, TDP-43, FUS, huntingtin, transthyretin, prion proteins, crystallin, immunoglobulin light chain, serum amyloid A, beta2-microglobulin, lysozyme, gelsolin, calcitonin, prolactin, IAPP or amylin, fibrinogen, rhodopsin, glucosylceramide, hemoglobin, DNA binding proteins, RNA binding proteins and other proteins with a potential for aggregation that have been implicated, or may be suspected of involvement in, other diseases, and other proteins studied in the laboratory setting and other proteins contained in pharmacologic preparations for which protein aggregation is assessed that include preparations of antibody against CD3, against CD4, against CD11, against CD19, against CD20, against CD22, against CD30, against CD38, against CD52, against CD79b, against PD-1, against PD-L1, against PD-L2, against CCR4, against IL-1, against IL-4R, against IL-5, against IL-5R, against IL-6, against IL-6 receptor, against IL-8, against IL-13, against IL-17, against IL-17 receptor, against IL-23, against IL-33, against IL-36 receptor, against HER2, against tissue factor, against CCR4, against EGFR, agasinst PDGRFa, against IFNAR1, against sclerostin, against von Willebrand factor, against C5, against IFNgamma, against FGF23, against Factor IXa, against Kalikrein, against complement C5, against BCMA, against angiopoietin-like 3, against TROP-2, against IGF-1R, against CGRP, SLAMF7, against PCSKg, against GD2, against Nectin-4, against P-selectin, against Ebola virus, against IgE, against GD2, against BLyS, against RANK-L, against B7-H3, against MASP-2, against LAG-3, against VEGF, against alpha4beta7 integrin, against C1s, against thymic stromal lymphopoietin, against folate receptor alpha, against RSV, against CTLA-4, against FcRn, against GPIIb/IIIa, against Ep/cAM, against endotoxin, against TNF, against G protein-coupled receptor 5D, against the COVID-19 spike protein and variants thereof, against Clostridium difficile enterotoxin B, protein preparations of IL-2, IL-7, IL-8, IL-10, IL-12, IL-15, IL-18, IL-23, IL-36, and combinations thereof.

3. The method according to claim 2, wherein the aggregated protein is a native, variant, mutant or posttranslationally modified form of: α-synuclein, tau, amyloid beta (Aβ42 and Aβ40), SOD1, TDP-43, FUS, huntingtin, transthyretin, prion proteins, crystallin, immunoglobulin light chain, serum amyloid A, beta2-microglobulin, lysozyme, gelsolin, calcitonin, prolactin, IAPP or amylin, fibrinogen, rhodopsin, glucosylceramide, hemoglobin, DNA binding proteins, RNA binding proteins and other proteins with a potential for aggregation that have been implicated, or may be suspected of involvement in, other diseases, and other proteins studied in the laboratory setting and other proteins contained in pharmacologic preparations for which protein aggregation is assessed that include preparations of antibody against CD3, against CD4, against CD11, against CD19, against CD20, against CD22, against CD30, against CD38, against CD52, against CD79b, against PD-1, against PD-L1, against PD-L2, against CCR4, against IL-1, against IL-4R, against IL-5, against IL-5R, against IL-6, against IL-6 receptor, against IL-8, against IL-13, against IL-17, against IL-17 receptor, against IL-23, against IL-33, against IL-36 receptor, against HER2, against tissue factor, against CCR4, against EGFR, agasinst PDGRFa, against IFNAR1, against sclerostin, against von Willebrand factor, against C5, against IFNgamma, against FGF23, against Factor IXa, against Kalikrein, against complement C5, against BCMA, against angiopoietin-like 3, against TROP-2, against IGF-1R, against CGRP, SLAMF7, against PCSKg, against GD2, against Nectin-4, against P-selectin, against Ebola virus, against IgE, against GD2, against BLyS, against RANK-L, against B7-H3, against MASP-2, against LAG-3, against VEGF, against alpha4beta7 integrin, against C1s, against thymic stromal lymphopoietin, against folate receptor alpha, against RSV, against CTLA-4, against FcRn, against GPIIb/IIIa, against Ep/cAM, against endotoxin, against TNF, against G protein-coupled receptor 5D, against the COVID-19 spike protein and variants thereof, against Clostridium difficile enterotoxin B, plus protein preparations of IL-2, IL-7, IL-8, IL-10, IL-12, IL-15, IL-18, IL-23, and IL-36 and others.

4. The method of claim 1, wherein the aggregated protein is present in a disease wherein protein aggregation is found to play a role.

5. The method of claim 4, wherein the disease is selected from the group consisting of Alzheimer's Disease (AD), Alzheimer's Disease Related Dementias, Parkinson's Disease (PD), Lewy Body Dementia, Huntington's Disease (HD), Prion Disease, Spongiform encephalopathies, Familial amyloid polyneuropathy, spinocerebellar ataxias, Creutzfeldt-Jakob disease, Fronto-temporal Dementia, amyotrophic lateral sclerosis (ALS), cardiac amyloidosis, chronic traumatic encephalopathy, primary and secondary systemic amyloidosis, Finnish Amyloidosis, medullary carcinoma of the thyroid, Senile Systemic Amyloidosis, prolactinomas, rheumatoid arthritis, hemodialysis-related amyloidosis, lysozyme systemic amyloidosis, hereditary renal amyloidosis, cataract disease, and diabetes mellitus type I and other diseases in which protein aggregation is found to play a role.

6. The method according to claim 1, wherein the microparticle is a sphere, bead, pellet, or non-planar shape composed of one or more of the following components: glass and modified or functionalized glass (e.g., carboxymethyldextran functionalized glass), plastics (including acrylics, polystyrene and copolymers of styrene and other materials, polypropylene, polyethylene, polybutylene, polyurethanes, Teflon®, polysaccharides, nylon, nitrocellulose, composite materials, ceramics, plastic resins, silica or silica-based materials including silicon and modified silicon (e.g., patterned silicon), carbon, metals, quartz (e.g., patterned quartz), inorganic glasses, plastics, optical fiber bundles, and other polymers including other compositions that are suitable.

7. The method according to claim 6, wherein the microparticle is composed of latex, polystyrene, silica, a magnetic material, a paramagnetic material, or any combination thereof.

8. The method according to claim 6, wherein the microparticle is spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape.

9. The method according to claim 8, wherein the microparticle is a polymer bead, a solid core bead, a carbon fiber bead, a hollow bead, a paramagnetic bead, or a microbead.

10. The method according to claim 6, wherein the surface of the microparticle is at least partially coated with a reactive moiety comprising an amino, a carboxyl, a thiol, or a hydroxyl reactive moiety or other molecule that serves the purpose.

11. The method according to claim 6, wherein the microparticle allows optical detection and do not appreciably fluoresce.

12. The method according to claim 1, wherein the microparticle is a paramagnetic bead.

13. The method according to claim 1, wherein the microparticle is a latex bead coated with aldehyde sulfate reactive moiety.

14. The method according to claim 6, wherein the greatest dimension of the microparticle ranges from 0.001 μm to 1000 μm, from 0.5 μm to 100 μm, from 0.1 μm to 20 μm, 20 μm or less, 15 μm or less, 10 μm or less, 5 μm or less, 1 μm or less, 0.75 μm or less, 0.5 μm or less, 0.4 μm or less, 0.3 μm or less, 0.2 μm or less, 0.1 μm or less, 0.01 μm or less, or 0.001 μm or less.

15. The method according to claim 1, wherein the first capture moiety is an antibody, or antigen binding fragment thereof, or a receptor, or a ligand, or an aptamer, or a polynucleotide that binds specifically to an aggregated protein or other aggregated species.

16. The method according to claim 15, wherein the first capture moiety binds to the same epitope or antigen, or amino acid sequence as the second capture moiety.

17. The method of claim 1, wherein the capture substrate non-specific binding sites are blocked by incubating the capture substrate with a non-specific blocker.

18. The method of claim 17, wherein the non-specific blocker is human serum albumin, bovine serum albumin, fetal bovine serum, non-fat milk proteins, casein, fish gelatin, polyethylene glycol, polyvinyl alcohol, or polyvinylpyrrolidone, other non-specific proteins, non-specific DNA or non-specific RNA.

19. The method of claim 1, wherein the second capture moiety is an antibody, or antigen binding fragment thereof, or a receptor, or a ligand, or an aptamer, or a polynucleotide that binds specifically to an aggregated protein or other aggregated species.

20. The method of claim 1, wherein the second capture moiety is conjugated to the detectable label.

21. The method of claim 1, wherein the second capture moiety is conjugated to a first binding partner, and the signaling moiety comprises a second binding partner conjugated to the detectable label.

22. The method of claim 21, wherein the first binding partner and the second binding partner comprises biotin, avidin or streptavidin.

23. The method of claim 1, wherein the second capture moiety is directly coupled to a detectable label, or coupled to biotin.

24. The method of claim 1, wherein the detectable label is a fluorescent label, a radiolabel, a chemiluminescent agent, and or a metal element label.

25. The method of claim 21, wherein the second binding partner of the signalling moiety is avidin, or streptavidin.

26. The method of claim 24, wherein determining the amount of detectable label present on the surface of the capture substrate comprises measuring the fluorescence, radioactivity, chemiluminescence, enzymatic product, heavy metal isotopes, or other signaling label of the capture substrate using a signal detection device.

27. The method of claim 1, wherein determining the amount of detectable label present on the surface of the capture substrate comprises counting the number of positive complexes.

28. The method according to claim 27, comprising determining a signal intensity of each detected complex.

29. The method according to claim 28, comprising determining a level of the protein aggregate based on the mean or median signal intensity of the detected complexes on a plurality of capture substrate microparticles which can be used to determine the size of protein aggregates.

30. The method of claim 1, wherein the test sample is: a patient sample, a laboratory reagent or a liquid pharmacological product, each test sample comprising one or more proteins.

31. The method of claim 30, wherein the test sample is a mammalian patient clinical sample selected from a whole blood sample, a serum sample, a plasma sample, a urine sample, an umbilical cord blood sample, a stool sample, a saliva sample, a sputum sample, a colostrum sample, a breast milk sample, a bone marrow sample, a lymph fluid sample, a peritoneal fluid sample, a pleural fluid sample, a joint fluid sample, a vitreous fluid sample, an inflammatory fluid sample, a tissue sample, a body cavity fluid sample, a cerebrospinal fluid (CSF) sample, tissue or fine needle biopsy samples; and also samples of free floating nucleic acids; gynecological fluids; skin swabs; vaginal swabs; oral swabs; nasal swabs; washings or lavages such as a ductal lavages or broncheoalveolar lavages; aspirates; scrapings; tissue biopsy specimens; surgical specimens; and other body fluids, secretions, and/or excretions; and/or cells therefrom.

32. The method of claim 30, wherein the test sample is obtained from a non-human primate, a mammalian animal, a vertebrate animal, a non-vertebrate animal, or a plant, and comprises a sample selected from a whole blood sample, a serum sample, a plasma sample, a urine sample, an umbilical cord blood sample, a stool sample, a saliva sample, a sputum sample, a colostrum sample, a breast milk sample, a bone marrow sample, a lymph fluid sample, a peritoneal fluid sample, a pleural fluid sample, a joint fluid sample, a vitreous fluid sample, an inflammatory fluid sample, a tissue sample, a body cavity fluid sample, a cerebrospinal fluid (CSF) sample, tissue or fine needle biopsy samples; and also samples of free floating nucleic acids; gynecological fluids; skin swabs; vaginal swabs; oral swabs; nasal swabs; washings or lavages such as a ductal lavages or broncheoalveolar lavages; aspirates; scrapings; tissue biopsy specimens; surgical specimens; and other body fluids, secretions, and/or excretions; and/or cells therefrom.

33. The method of claim 30, wherein the test sample is a laboratory reagent or pharmacological product comprising at least one protein or other aggregating species.

34. The method of claim 30, wherein the pharmacological product is a liquid sample comprising: insulin, an antibody, a vaccine, a CAR-T, a hormone, a cytokine, a chemokine, a growth factor, an enzyme, serum albumin, a DNA binding protein, an RNA binding protein, serum, plasma, erythropoietin (EPO), a receptor, a ligand, a blood coagulation factor, an Fc fusion recombinant protein, and combinations thereof.

35. The method of claim 33, wherein the test sample is a pharmacological product containing at least one protein, or other aggregating species, and is formulated for parenteral or oral or nasal or rectal administration.

36. The method of claim 1, wherein the aggregated protein comprises oligomers, multimers, protofibrils, fibrils or combinations thereof.

37. A composition, comprising
(a) a first component comprising a plurality of capture substrate microparticles, each microparticle conjugated to a first capture moiety antibody or antigen binding fragment thereof, or receptor, or ligand, or aptamer, an amino acid sequence, or lipid, or DNA nucleotide sequence, or RNA nucleotide sequence; the first capture moiety antibody or antigen binding fragment thereof, or receptor, or ligand, or aptamer, an amino acid sequence, or lipid, or DNA nucleoside sequence, or RNA nucleoside sequence, operable to bind to a single epitope or antigen, or sequence of amino acids of an aggregated protein involved in a protein aggregation mediated disease, or other aggregating species involved in disease.
(b) a second component comprising a second capture moiety that comprises an antibody or antigen binding fragment thereof, a receptor, a ligand, an aptamer, an amino acid sequence, a lipid, or DNA nucleotide sequence, or RNA nucleotide sequence, as the first capture moiety, wherein the second capture moiety is conjugated to a signalling moiety that comprises at least one of: a detectable label and a first binding partner that is operable to bind specifically to a detectable label that is coupled to a second binding partner, and
(c) optionally, a third component a detectable label coupled to a second binding partner, wherein the first and second binding partners bind specifically.

38. The composition of claim 37, wherein the first component of the composition is present in a microtiter plate well or first liquid receptacle, and the second component of the composition is present in a second liquid receptacle, and the composition of claim 36 wherein the components are brought together in a continuous flow microfluidics system.

39. A system, comprising: a processor; and
a non-transitory computer readable medium comprising instructions that cause the processor to:
count a number of positive aggregate protein capture complexes present in a test sample, wherein each of the positive aggregate protein capture complexes comprise a first capture moiety, an aggregate protein, a second capture moiety, a signalling moiety and a fluorescently labeled detectable label;
determine the total number of aggregate protein capture complexes acquired by a detection device; calculate the percentage of positive aggregate protein capture complexes among the total number of aggregate protein capture complexes; and
determine the number and/or proportion of positive aggregate protein capture complexes that comprised the test sample.

40. The system of claim 39, wherein the non-transitory computer readable medium further comprises instructions that cause the processor to:
determine a mean or a median fluorescence intensity of the positive aggregate protein capture complexes acquired by the detection system; and
determine a level of the aggregate proteins present in the test sample.

41. The system of claim 39, wherein the system is a flow cytometry system.

42. The system of claim 40, wherein the system is a mass cytometry system, or a spectrometer, or a spectrofluorometer.

43. A kit, comprising:
a plurality of microparticles comprising a first capture moiety that specifically binds an aggregate protein of interest; and
a composition comprising a second capture moiety that specifically binds to the aggregate protein, wherein the second capture moiety is coupled to a signalling moiety, wherein the first and second capture moieties bind to the same epitope or antigen present on the aggregated protein.

44. The kit of claim 43, wherein the first capture moiety and the second capture moiety comprise an antibody, or antigen binding fragment thereof or a polynucleotide, wherein the first capture moiety and the second capture moiety bind specifically to an aggregated protein.

45. The method according to claim 15, wherein the first capture moiety binds to the same epitope or antigen, or amino acid sequence of the aggregated protein as the second capture moiety.

46. The kit of claim 43, wherein the signalling moiety comprises a detectable label.

47. The kit of claim 43, wherein the second capture moiety is an antibody, or antigen binding fragment thereof, a receptor, a ligand, an aptamer, a lipid, a polynucleotide (DNA or RNA), or a sequence of amino acids that binds specifically to the aggregate bound by the first capture moiety.

48. The kit of claim 43, wherein the second capture moiety is conjugated to the detectable label.

49. The kit of claim 43, wherein the second capture moiety is conjugated to a first binding partner, and the signaling moiety comprises a second binding partner conjugated to the detectable label.

50. The kit of claim 43, wherein the first binding partner and the second binding partner comprises biotin, avidin or streptavidin.

51. The kit of claim 43, wherein the second capture molecule is directly coupled to a fluorescent molecule, or coupled to biotin.

52. The kit of claim 43, wherein the detectable label is a fluorescent label, a radiolabel, a luminescent agent, and a metal element label.

53. The kit of claim 43, wherein the second binding partner of the signalling moiety is avidin, or streptavidin.

54. The kit of claim 53, further comprising a blank microtiter plate having 6, 12, 24, 48, 96, 384, 1536, or 3456 sample wells.

55. The kit of claim 43, further comprising instructions for capturing aggregate proteins with the microparticles from a test sample.

56. The kit of claim 55, wherein the instructions are for detecting the aggregate protein by flow cytometry.
